(19) United States
(12) Patent Application Publication
(10) Pub. No.: US 2023/0314432 A1
(43) Pub. Date: Oct. 5, 2023

(54) COMPOSITIONS, METHODS, AND SYSTEMS FOR DETECTING IMMUNE RESPONSE

(71) Applicant: COVID DIAGNOSTICS LTD., Oxford (GB)

(72) Inventor: Colin George Ferrett, Oxford (GB)

(21) Appl. No.: 18/020,775

(22) PCT Filed: Jul. 9, 2021
(86) PCT No.: PCT/IB2021/000463

§ 371 Date: Feb. 10, 2023

Related U.S. Application Data
(60) Provisional application No. 63/050,526, filed on Jul. 10, 2020.

(51) Int. Cl. G01N 33/569; C12Q 1/70; C12Q 1/6876

(52) U.S. Cl. CPC G01N 33/56983; C12Q 1/701; C12Q 1/6876; G01N 2333/165

(57) ABSTRACT

Described herein are compositions and methods for detecting immune response to pathogens. Also described herein are methods and systems utilizing the compositions for detecting adaptive immunity.

### CROSS-REFERENCE

This application claims the benefit of U.S. Provisional Application No. 63/050,526 filed Jul. 10, 2020, which is incorporated by reference herein in its entirety.

### BACKGROUND

Viral infections can cause severe diseases and even deaths. An example of the ongoing challenges presented by viral infection is the coronavirus disease of 2019 (COVID-19), caused by severe acute respiratory syndrome coronavirus 2 (SARS-CoV-2). Vaccines or treatment options often do not exist for some existing or new viruses, or may require significant time and resources to develop. A fast and reliable test for immune response will release individuals from the constraints of physical distancing and reduce cost for public health measures.

### SUMMARY

Provided herein are point of need compositions, kits, and systems for detecting immune response to one or more pathogens, e.g. viruses, in a subject and methods for using same, as well as devices to perform methods described herein.

Provided herein, in an aspect are containers for detecting immune responses to specific pathogens by a subject. In an embodiment, provided herein is a container for detecting an immune response to a pathogen by a subject, wherein the container comprises an antigen of the pathogen and an anticoagulation agent, and wherein the antigen comprises a specific T cell recognized epitope of the pathogen.

In some embodiments, the antigen is lyophilized. In another embodiment, the antigen is in a solution.

In some embodiments, the container further comprises a sample obtained from the subject.

In some embodiments, the sample comprises PBMCs, CD8 T cells, NK cells, CD4 T cells, B cells, or any combination thereof.

In some embodiments, the sample comprises a body fluid. In some embodiments, the sample comprises saliva. In some embodiments, the sample comprises whole blood.

In some embodiments, the subject is a human.

In some embodiments, the container is used in detecting an immune response to the pathogen for a subject that exhibits symptoms of a condition associated with exposure to the pathogen. In some embodiments, the container is used in detecting an immune response to the pathogen for a subject that exhibits symptoms of a condition associated with exposure to the pathogen. The container is used in detecting an immune response to the pathogen for a subject suspected to have been exposed to the pathogen. In some embodiments, the container is used in incubating whole blood for at least 30 minutes. In some embodiments, the container is used in incubating whole blood for at least 6 hours. In some embodiments, the container is used in incubating whole blood for at least 24 hours.

In some embodiments, the antigen is stable for at least 30 days. In some embodiments, the antigen is stable for at least 6 months. In some embodiments, the antigen is stable for at least a year.

In some embodiments, the anticoagulation agent is heparin, citrate, unfractionated heparin, bivalirudin, or hirudin. In some embodiments, the anticoagulation agent is lithium heparin.

In some embodiments, the pathogen is a mammalian pathogen. In some embodiments, the pathogen is a human pathogen. In some embodiments, the pathogen is an animal pathogen. In some embodiments, the pathogen is a viral pathogen and the antigen is from a disease causing virus. In some embodiments, the pathogen is a mammalian viral pathogen. In some embodiments, the pathogen is a human or animal viral pathogen.

In some embodiments, the antigen is from a DNA virus. In some embodiments, the antigen is from a RNA virus. In some embodiments, the antigen is from a retrovirus. In some embodiments, the antigen is from a Coronavirus, a Rhabdovirus, a paramyxovirus, an influenza virus, a respiratory syncytial virus, a cytomegalovirus, a Hepatitis virus or a orthomyxovirus. In some embodiments, the antigen is from a SARS-Cov2 virus. In some embodiments, the antigen comprises a polypeptide comprising a sequence with at least 80% identity to SEQ ID NO: 1. In some embodiments, the antigen comprises a polypeptide comprising a sequence with at least 85% identity to SEQ ID NO: 1. In some embodiments, the antigen comprises a polypeptide comprising a sequence with at least 90% identity to SEQ ID NO: 1.

In some embodiments, the antigen comprises a polypeptide comprising a sequence with at least 80% identity to SEQ ID NO: 2. In some embodiments, the antigen further comprises a polypeptide comprising a sequence with at least 85% identity to SEQ ID NO: 2. In some embodiments, the antigen further comprises a polypeptide comprising a sequence with at least 90% identity to SEQ ID NO: 2. In some embodiments, the antigen comprises a peptide comprising 5-14 consecutive amino acids of SEQ ID NO: 1. In some embodiments, the antigen comprises a peptide comprising 5-14 consecutive amino acids of SEQ ID NO: 2. In some embodiments, the antigen comprises a peptide comprising at least 15 consecutive amino acids of SEQ ID NO: 1. In some embodiments, the antigen comprises a peptide comprising at least 15 consecutive amino acids of SEQ ID NO: 2.

In some embodiments, the antigen comprises at least two specific T cell recognized epitopes of the virus. In some embodiments, the T cell recognition is by a CD4+ T cell. In some embodiments, the T cell recognition is by a CD8+ T cell.

In some embodiments, the container further comprises a second antigen comprising a second specific T cell recognized epitope of the pathogen. In some embodiments, the container further comprises a second antigen of a second pathogen, and wherein the second antigen comprises a specific T cell recognized epitope of the second pathogen.

In some embodiments, the second pathogen is a human pathogen. In some embodiments, the pathogen is a viral pathogen and second the antigen is from a disease causing virus. In some embodiments, the second pathogen is a mammalian viral pathogen. In some embodiments, the second pathogen is a human or animal viral pathogen.

In some embodiments, the container further comprises one or more of a sugar, an amino acid, and a vitamin. In some embodiments, the sugar is a glucose.

In some embodiments, the container further comprising an aluminum adjuvant. In some embodiments, the aluminum adjuvant and the antigen are at a ratio of 1:1 to 3:1 by weight, wherein weight of the aluminum adjuvant is as measured by weight of aluminum ion.

Provided herein, in another embodiment is a container for detecting an immune response to a SARS-Cov2 virus in a subject, wherein the container comprises lithium heparin, glucose, an aluminum adjuvant, and a SARS-Cov2 antigen that comprises the sequence of SEQ ID NO: 2, wherein the aluminum adjuvant and the SARS-Cov2 antigen are at a ratio of 1:1 to 3:1 by weight, wherein weight of the aluminum adjuvant is as measured by weight of aluminum ion.

In another aspect, provided herein is a kit for detecting an immune response to a virus in a subject comprising the container and a second container comprising an anticoagulation agent and not the antigen. In some embodiments, the kit further comprises a detection agent for detecting an immune cell response to the antigen, wherein the detection agent quantifies an indicator of the immune cell response. In some embodiments, the indicator comprises an amount of an immune-response analyte. In some embodiments, the indicator comprises a ratio of two or more immune-response analytes. In some embodiments, the immune-response analyte or the two or more immune-response analytes are immunoproteins. In some embodiments, the immune-response analyte or the two or more immune-response analytes are nucleic acids encoding the immunoproteins.

In some embodiments, the indicator is an immune cell proliferation indicator. In some embodiments, the immune-response analyte is an IFN-gamma protein or a nucleic acid encoding an IFN-gamma protein.

In some embodiments, the kit further comprises one or more primers that hybridize with the nucleic acid encoding the IFN-gamma protein.

In some embodiments, the kit further comprises a lysis agent. In some embodiments, the kit further comprises instructions for quantifying the immune-response analytes.

In some embodiments, the kit further comprises a set of primers that hybridize with a nucleic acid of the pathogen.

In some embodiments, the set of primers hybridize with a nucleic acid encoding a SARS-CoV-2 protein.

Provided herein, in another embodiment, is a kit for detecting an immune response to a SARS-Cov2 virus by a subject, wherein the kit comprises a) a first container, comprising lithium heparin, glucose, an aluminum adjuvant, and a SARS-Cov2 antigen that comprises the peptide sequence of SEQ ID NO: 2, b) a second container comprising lithium heparin, glucose, and not the SARS-Cov2 antigen, wherein the first and second containers are sterile glass or polycarbonate containers, and c) a set of primers that comprise a sequence selected from Table 2, wherein the aluminum adjuvant and the SARS-Cov2 antigen are at a ratio of 1:1 to 3:1 by weight, wherein weight of the aluminum adjuvant is as measured by weight of aluminum ion.

In one aspect, provided herein is a method for detecting an immune response to a pathogen by a subject, comprising i) incubating a blood sample from the subject in a container comprising an antigen and an anticoagulation agent, wherein the antigen comprises a specific T cell recognized epitope of the pathogen, and ii) incubating a second blood sample from the subject in a second container comprising the anticoagulation agent in (i) and not the antigen, iii) quantifying an amount of an immune-response analyte in the container and the second container, iv) comparing the amount of the immune-response analyte in the container and the second container to determine whether the subject has an immune response to the pathogen.

In some embodiments, the quantifying in iii) comprises using mass spectrometry to determine the amount of the immune-response analyte. In some embodiments, the immune-response analyte is a protein.

In some embodiments the method further comprises isolating the immune response analyte.

In some embodiments, the method further comprises isolating a nucleic acid encoding the immune response analyte.

In some embodiments, the method further comprises isolating total RNA from the container.

In some embodiments, the quantifying in (iii) comprises Western blot, mass spectrometry assay, UV spectrometry assay, aptamer based assay, or colorimetric assay. In some embodiments, the quantifying in (iii) comprises amplifying the nucleic acid encoding the immune-response marker. In some embodiments, the quantifying in (iii) comprises determining the amount of the nucleic acid encoding the immune-response marker with loop-mediated isothermal proliferation (LAMP) assay, RNA seq, or quantitative RT-PCR. In some embodiments, the quantifying in (ii) further comprises analysis using a nanopore technology.

In some embodiments, the container further comprises a second antigen comprising a specific T cell recognized epitope of a second pathogen, wherein the second container does not comprise the second antigen, and wherein the comparing in (iv) comprises comparing the amount of the immune-response analyte in the container and the second container to determine whether the subject has an immune response to the second pathogen.

In some embodiments, the method further comprises v) quantifying an amount of a second immune-response analyte in the container and the second container, and vi) comparing the amount of the second immune-response analyte in the container and the second container to determine whether the subject has an immune response to the pathogen.

In some embodiments, the comparing in vi) comprises comparing a ratio of the immune-response analyte to the second immune-response analyte in the container and the second container to determine whether the subject has an immune response to the pathogen.

In some embodiments, the method further comprises vii) detecting the pathogen or the second pathogen in the sample, wherein the detecting comprises amplifying a nucleic acid of the pathogen or the second pathogen.

In some embodiments, the amplifying comprises LAMP assay, RNA-Seq, or RT-PCR. In some embodiments, the amplifying is with a set of primers, wherein the set of primers hybridize with the nucleic acid. In some embodiments, the set of primers hybridize with a nucleic acid encoding a SARS-CoV-2 protein.

Provided herein, in another embodiment is a method for detecting an immune response to a SARS-Cov2 virus by a subject, comprising: i) incubating a whole blood sample from the subject in a container comprising lithium heparin, glucose, an aluminum adjuvant, and a specific SARS-Cov2 antigen for at least 24 hours, ii) incubating a second whole blood sample from the subject in a second container comprising lithium heparin, glucose, an aluminum adjuvant, and not a SARS-Cov2 antigen, and iii) quantifying an amount of an IFN-gamma mRNA in the container and the second container to determine whether the subject has an immune response to the SARS-Cov2 virus, wherein the aluminum adjuvant and the SARS-Cov2 antigen are at a ratio of 1:1 to 3:1 by weight, wherein weight of the aluminum adjuvant is as measured by weight of aluminum ion, wherein the SARS-Cov2 antigen comprises the peptide sequence of SEQ ID NO: 2, and wherein the quantifying in (iii) comprises amplifying the IFN-gamma mRNA with loop-mediated isothermal proliferation (LAMP) assay using primers selected from Table 2.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A-FIG. 1B depict exemplary results of analysis using whole blood samples. FIG. 1A depicts a colorimetric assay of LAMP assay product. FIG. 1B shows the quantification of the amplified interferon-gamma signal from the LAMP Assay using convalescent blood in the presence and absence of antigen.

FIG. 2A-FIG. 2B show quantification of IFN-gamma expression using RT-PCR or LamPORE.

FIG. 2A show the quantification of IFN-gamma expression by RT-PCR using convalescent blood from patients previously exposed or blood from patients not previously exposed to COVID. A mosaic CoV-2 antigen was used. FIG. 2B shows the quantification of IFN-gamma expression by LAMP technology combined with Nanopore technology (LamPORE) using convalescent blood from patients previously exposed or blood from patients not previously exposed to COVID.

FIG. 3 demonstrates the fold change expression of interferon-gamma using positive convalescent patient blood on different CoV-2 antigens: N1, N2b, Spike, Mosaic, and mitogen antigens.

FIG. 4 shows the quantification of interferon-gamma expression after antigen stimulation in COVID positive convalescent patients, in which whole blood was stimulated with a control or CoV-2 mosaic antigen for 2, 4, or 6 hours.

FIG. 5 shows the quantification of interleukin-2 (IL-2) expression after antigen stimulation in COVID positive convalescent patients, in which whole blood was stimulated with a control or CoV-2 mosaic antigen for 2, 4, or 6 hours.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms, “clinic,” “clinical setting,” “laboratory” or “laboratory setting” refer to a hospital, a clinic, a pharmacy, a research institution, a pathology laboratory, a or other commercial business setting where trained personnel are employed to process and/or analyze biological and/or environmental samples. These terms are contrasted with point of care, a remote location, a home, a school, and otherwise non-business, non-institutional setting.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing is relative or absolute. “Detecting the presence of” can include determining the amount of something present in addition to determining whether it is present or absent depending on the context.

The terms “subject,” or “individual” are often used interchangeably herein. A “subject” may be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. A subject may or may not have been exposed to a pathogen of interest as described herein, and may by symptomatic or symptomatic of a disease or condition associated with infection of or exposure to a pathogen as described herein. In some embodiments, a subject is suspected to have been exposed to a pathogen, e.g. a virus. In some embodiments, a subject has been exposed to an antigen or a protein representative or cross-reacts with antigens of a particular pathogen, e.g. a virus. In some embodiments, a subject has one or more symptoms that are indicative of a disease or condition associated with infection of or exposure to a pathogen as described herein. In some embodiments, the subject is currently infected by a pathogen, e.g. a virus described herein. In some embodiments, the subject is previously infected by a pathogen described herein. In some embodiments, a subject is a carrier of a virus described herein. In some embodiments, a subject is a carrier of fragments or remnants of a virus described herein. In some instances, a subject is carrier of adaptive immunity stemmed from previously or currently being infected by a virus described herein. In some embodiments, a subject is a carrier of adaptive immunity stemmed from previous or current exposure to a different virus or pathogen other than a virus or pathogen of interest.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the terms “polypeptide,” “peptide” and “protein” can be used interchangeably herein in reference to a polymer of amino acid residues. A protein can refer to a full-length polypeptide as translated from a coding open reading frame, or as processed to its mature form, while a polypeptide or peptide can refer to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

As used herein, the terms “fragment,” or equivalent terms can refer to a portion of a protein that has less than the full length of the protein and optionally maintains the function of the protein. Further, when the portion of the protein is blasted against the protein, the portion of the protein sequence can align, for example, at least with 80% identity to a part of the protein sequence.

Any systems, methods, and platforms described herein are modular and not limited to sequential steps. Accordingly, terms such as “first” and “second” do not necessarily imply priority, order of importance, or order of acts.

### Antigens

Described herein, in some embodiments, are compositions comprising one or more antigens, epitopes, or a fragments thereof derived from a pathogen, e.g., a virus.

As used herein, an antigen refers to any molecule or agent that is capable of stimulating or re-stimulating an immune response by an immune cell in vivo or ex vivo. In some embodiments, an antigen is bound by an antibody. In some embodiments, an antigen is recognized and bound by a major histocompatibility complex (MHC) and can be presented to a T-cell receptor. In some embodiments, an antigen is an immunogen. Antigens include but are not limited to peptides, proteins, haptens, allergens or toxins or any naturally occurring or synthetic molecule or parts thereof. In some embodiments, an antigen is derived from a pathogen or a portion or lysate thereof. In some embodiments, an antigen comprises one or more proteins or peptides derived from a pathogen or a portion or lysate thereof. For example, an antigen may comprise one or more proteins or peptides derived from viral protein or a portion. In some embodiments, an antigen comprises a protein or peptide derived from different structural or functional proteins of a pathogen, e.g. a virus. In some embodiments, an antigen comprises a protein or peptide derived from structural or functional proteins of multiple strains or types of a pathogen, e.g. two or more strains or two or more serotypes of a virus. In some embodiments, an antigen comprises a protein or peptide derived from different structural or functional proteins of more than one pathogens, e.g. two or more nucleocapsid proteins of two or more viruses. a pathogen or a portion or lysate thereof. In some embodiments, an antigen comprises one or more proteins or peptides derived from a pathogen or a portion or lysate thereof. In some embodiments, an antigen comprises peptides, proteins, including glycoproteins, carbohydrates, phospholipids, phosphoproteins, phospholipoproteins, or any fragments thereof.

An antigen can comprise a peptide of various lengths. In some embodiments, an antigen as described herein comprises at least 1, 2, 3, 4, 5, 6, 7, 8, 9, 10, or more amino acids. In some embodiments, an antigen comprises one or more peptides having a length of 5 to 100 amino acids, 5 to 20 amino acids, 5 to 30 amino acids, 5 to 40 amino acids, 5 to 50 amino acids, 6 to 50 amino acids, 7 to 50 amino acids, 8 to 50 amino acids, 9 to 50 amino acids, or 10 to 50 amino acids. In some embodiments, an antigen is provided by a set of peptides from one or more different full length or part length peptides, for example, one or more overlapping or non-overlapping peptides. The one or more overlapping or non-overlapping peptides may cover the entire length of or a part of a naturally occurring protein antigen.

In some embodiments, the antigen comprises one or more peptides of about 1 to up to 5, up to 6, up to 7, up to 8, up to 9, up to 10, up to 20, up to 30, up to 50, up to 100, up to 150, up to 200, up to 250, up to 300, up to 350, or up to 400 or more amino acids. In some embodiments, the antigen comprises one or more peptides recognized by a CD8+ cytotoxic T-cell. Such one or more peptides may be of a length of equal to or less than 15, 14, 13, 12, 11, 10, or 9 amino acids. In some embodiments, the antigen comprises a peptide recognized by a CD8+ cytotoxic T-cell with a length of 7-14 amino acids, 7-13 amino acids, 8 to 12 amino acids, 8-11 amino acids or 8 to 10 amino acids. In some embodiments, an antigen comprises one or more of a plurality of peptides recognized by a CD8+ cytotoxic T cell. In some embodiments, the plurality of peptides encompass all or a part of a protein antigen such as a viral protein antigen, or a combination of multiple protein antigens, e.g., one or more of structural or functional proteins of a coronavirus. In some embodiments, the antigen comprises one or more peptides recognized by a CD4+ T-cell. Such one or more peptides may be of a length of equal to or more than 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. In some embodiments, the antigen comprises a peptide recognized by a CD8+ cytotoxic T-cell with a length of 7-14 amino acids, 7-13 amino acids, 8 to 12 amino acids, 8-11 amino acids or 8 to 10 amino acids. In some embodiments, an antigen comprises one or more of a plurality of peptides recognized by a CD8+ cytotoxic T cell. In some embodiments, the plurality of peptides encompass all or a part of a protein antigen such as a viral protein antigen, or a combination of multiple protein antigens, e.g., one or more of structural or functional proteins of a coronavirus.

In some embodiments, the antigen comprises a nucleocapsid protein, a E protein, or a S protein of a coronavirus (CoV). In some embodiments, the antigen comprises the full length of the nucleocapsid protein of a virus, e.g. a coronavirus. In some embodiments, the antigen comprises from 1 to up to 5, up to 6, up to 7, up to 8, up to 9, up to 10, up to 20, up to 30, up to 50, up to 100, up to 200, or up to 400 or more amino acids of the full length nucleocapsid protein. In some embodiments, the antigen comprises the full length of a E protein of a coronavirus. In some embodiments, the antigen comprises from 1 to up to 5, up to 6, up to 7, up to 8, up to 9, up to 10, up to 20, up to 30, up to 50, up to 75 or more amino acids of a full length E protein of a coronavirus. In some embodiments, the antigen an comprise the full length of a S protein of a coronavirus. In some embodiments, the antigen comprises from 1 to up to 5, up to 6, up to 7, up to 8, up to 9, up to 10, up to 20, up to 30, up to 50, up to 100, up to 200, up to 400, up to 500, up to 1000, up to 2000, up to 5000 or more amino acids of a full length S protein of a coronavirus.

An antigen can comprise one or more peptides. In some embodiments, an antigen comprises multiple peptides or fragments that are not the same. In some embodiments, an antigen as described herein comprises at least 1, 2, 3, 4, 5, 6, 7, 8, 9, 10, or more peptides or fragments that are not the same. In some embodiments, the peptides or polypeptides are from the same strain of a virus or from different strains of a virus. In some embodiments, the peptides or polypeptides are from different viruses. In some embodiments, at least one polypeptide or fragment stimulates T cell proliferation in vivo or ex vivo. In some embodiments, at least one polypeptide or fragment induces an immune response in a subject. In some embodiments, the at least one polypeptide or fragment induces immune response in a subject, who has developed adaptive immunity against the at least one polypeptide or fragment.

In some embodiments, an antigen described herein comprises a polypeptide derived from a viral protein or peptide. In some embodiments, the polypeptide is derived from is a fragment of the viral peptide. For example, an antigen may comprise a peptide derived from a viral capsid protein, a nucleocapsid protein, a viral core protein, a viral envelope protein, a viral non-structural protein, a viral replicase, a viral replication enzyme, a viral matrix protein, a viral polymerase, a viral fusion protein, or a fragment or variant thereof. In some embodiments, an antigen comprises a peptide derived from a nucleocapsid protein, a spike protein, a E protein, a S protein, a N protein, or a M protein of a corona virus (CoV). In some embodiments, an antigen comprises a peptide derived from a nucleocapsid protein, a spike protein, a E protein, a S protein, a N protein, or a M protein of a SARS-Cov-2.

In some embodiments, an antigen described herein comprises a synthetic polypeptide. For example, an antigen may comprise a synthetic polypeptide derived from multiple proteins of a pathogenic origin, for example, multiple proteins or a virus or multiple proteins from different viruses. In some embodiments, an antigen comprises one or more synthetic polypeptides that are mosaics derived from multiple proteins of a pathogenic origin. In some embodiments, the mosaic peptide comprises an amino acid sequence at least 50%, 60%, 70%, 80%, 90%, 95%, or 99% identical to a viral capsid protein. In some embodiments, the mosaic peptide comprises an amino acid sequence at least 50%, 60%, 70%, 80%, 90%, 95%, or 99% identical to a nucleocapsid protein. In some embodiments, the mosaic peptide comprises an amino acid sequence at least 50%, 60%, 70%, 80%, 90%, 95%, or 99% identical to a viral spike protein. In some embodiments, the mosaic peptide comprises an amino acid sequence at least 50%, 60%, 70%, 80%, 90%, 95%, or 99% identical to a nucleocapsid protein of a SARS-Cov-2 virus. In some embodiments, the mosaic peptide comprises an amino acid sequence at least 50%, 60%, 70%, 80%, 90%, 95%, or 99% identical to a S protein of a SARS-Cov-2 virus. In some embodiments, the mosaic peptide comprises an amino acid sequence at least 50%, 60%, 70%, 80%, 90%, 95%, or 99% identical to a M protein of a SARS-Cov-2 virus. In some embodiments, the mosaic peptide comprises an amino acid sequence at least 50%, 60%, 70%, 80%, 90%, 95%, or 99% identical to a nucleocapsid protein of a SARS-Cov-2 virus. In some embodiments, the mosaic peptide comprises an amino acid sequence at least 50%, 60%, 70%, 80%, 90%, 95%, or 99% identical to a N protein of a SARS-Cov-2 virus. In some embodiments, the mosaic peptide comprises an amino acid sequence at least 50%, 60%, 70%, 80%, 90%, 95%, or 99% identical to a nucleocapsid protein of a SARS-Cov-2 virus. In some embodiments, the mosaic peptide comprises an amino acid sequence at least 50%, 60%, 70%, 80%, 90%, 95%, or 99% identical to a receptor binding domain of a spike protein of a SARS-Cov-2 virus. In some embodiments, the mosaic peptide comprises an amino acid sequence at least 50%, 60%, 70%, 80%, 90%, 95%, or 99% identical to SEQ ID NO: 1. In some embodiments, the mosaic peptide comprises an amino acid sequence at least 50%, 60%, 70%, 80%, 90%, 95%, or 99% identical to SEQ ID NO: 2.

An antigen can comprise one or more epitopes of a specific pathogen, e.g. a virus. In some embodiments, an antigen comprises 1, 2, 3, 4, 5, 6, 7, 8, 9, 10, 11, 12, 13, 14, 15, 16, 17, 18, 19, 20, 21, 22, 23, 24, 25, 26 or more epitopes of a virus. In some embodiments, an antigen comprises at least one polypeptide derived from a viral protein or peptide encoded by a viral genome or viral genes. In some embodiments, the at least one polypeptide is encoded and derived from any open reading frame of the viral genome. Exemplary viral peptide includes orf1a, orf1ab, spike protein (S protein), 3a, 3b, envelope protein (E protein), matrix protein (M protein), p6, 7a, 7b, 8b, 9b, nucleocapsid protein (N protein), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRp), nsp13 (RNA 5′-triphosphatase), nsp14 (3′-to-5′ exonuclease), nsp15 (endoRNAse), and nsp16 (2′-O-ribose methyltransferase).

In some embodiments, the at least one polypeptide is derived from a nucleocapsid protein of a coronavirus. In embodiments, the at least one polypeptide comprises more than one peptides derived from more than two nucleocapsid proteins of a corona virus. In some embodiments, the more than one nucleocapsid proteins are derived from the same strain of coronavirus or from different strains of the coronavirus. In some embodiments, the at least one polypeptide comprises at least or equal to 2, 3, 4, 5, 6, 7, 8, 9, or 10 peptides derived from a nucleocapsid protein, wherein each of the peptides is different. In some embodiments, the at least one polypeptide comprises at most or equal to 2, 3, 4, 5, 6, 7, 8, 9, or 10 peptides derived from a nucleocapsid protein, wherein each of the peptides is different. In some embodiments, the at least one polypeptide comprises 9, 10, 11, 12, 13, 14, 15, 16, 17, 18, 19, 20, 21, 22, 23, 24, or 25 consecutive amino acids derived from a nucleocapsid protein of a corona virus. In some embodiments, the at least one polypeptide is derived from a spike protein of a coronavirus. In embodiments, the at least one polypeptide comprises more than one peptides derived from more than two spike proteins of a corona virus. In some embodiments, the more than one spike proteins are derived from the same strain of coronavirus or from different strains of the coronavirus. In some embodiments, the at least one polypeptide comprises at least or equal to 2, 3, 4, 5, 6, 7, 8, 9, or 10 peptides derived from a spike protein, wherein each of the peptides is different. In some embodiments, the at least one polypeptide comprises at most or equal to 2, 3, 4, 5, 6, 7, 8, 9, or 10 peptides derived from a spike protein, wherein each of the peptides is different. In some embodiments, the at least one polypeptide comprises 9, 10, 11, 12, 13, 14, 15, 16, 17, 18, 19, 20, 21, 22, 23, 24, or 25 consecutive amino acids derived from a spike protein of a corona virus. In some embodiments, the at least one polypeptide is derived from a spike protein of a coronavirus. In embodiments, the at least one polypeptide comprises more than one peptides derived from more than two E proteins of a corona virus. In some embodiments, the more than one E proteins are derived from the same strain of coronavirus or from different strains of the coronavirus. In some embodiments, the at least one polypeptide comprises at least or equal to 2, 3, 4, 5, 6, 7, 8, 9, or 10 peptides derived from a E protein, wherein each of the peptides is different. In some embodiments, the at least one polypeptide comprises at most or equal to 2, 3, 4, 5, 6, 7, 8, 9, or 10 peptides derived from a E protein, wherein each of the peptides is different. In some embodiments, the at least one polypeptide comprises 9, 10, 11, 12, 13, 14, 15, 16, 17, 18, 19, 20, 21, 22, 23, 24, or 25 consecutive amino acids derived from a E protein of a corona virus. In some embodiments, the at least one polypeptide is derived from a receptor binding domain of a spike protein of a coronavirus. In embodiments, the at least one polypeptide comprises more than one peptides derived from more than two receptor binding domain of a spike proteins of a corona virus. In some embodiments, the more than one receptor binding domain of a spike proteins are derived from the same strain of coronavirus or from different strains of the coronavirus. In some embodiments, the at least one polypeptide comprises at least or equal to 2, 3, 4, 5, 6, 7, 8, 9, or 10 peptides derived from a receptor binding domain of a spike protein, wherein each of the peptides is different. In some embodiments, the at least one polypeptide comprises at most or equal to 2, 3, 4, 5, 6, 7, 8, 9, or 10 peptides derived from a receptor binding domain of a spike protein, wherein each of the peptides is different. In some embodiments, the at least one polypeptide comprises 9, 10, 11, 12, 13, 14, 15, 16, 17, 18, 19, 20, 21, 22, 23, 24, or 25 consecutive amino acids derived from a receptor binding domain of a spike protein of a corona virus. In some embodiments, the corona virus is a SARS-CoV. In some embodiments, the corona virus is a MERS-CoV. In some embodiments, the corona virus is a SARS-CoV-2.

### SARS-CoV-2

The novel coronavirus (COVID-19) world-wide epidemic continues to cause large scale disruption to society. Severe acute respiratory syndrome coronavirus-2 (SARS CoV-2) is the virus that caused the global pandemic that was first reported on Dec. 31, 2019. SARS CoV-2 is an RNA virus and its RNA genome is 30 kb in length, and belongs to the realm Riboviria, order Nidovirales, suborder Cornidovirineae, family Coronaviridae, subfamily Orthocoronavirinae, genus Betacoronavirus (lineage B), subgenus Sarbecovirus, and the species Severe acute respiratory syndrome-related coronavirus.

SARS CoV-2 (NCBI Reference Sequence: NC_045512.2) comprise genomic sequences similar to the genome of the coronavirus that caused the SARS epidemic in 2003 (SARS CoV, NCBI Reference sequence: NC_004718.3) SARS-Cov-2 proteins include the spike protein, envelope protein, membrane protein, nucleocapsid protein, 3CL protease, papain like protease, RNA polymerase, and helicase protein. Exemplary sequences of SARS CoV-2 structural and functional proteins are provided in Table 1 below.

TABLE 1
SARS-CoV-2 protein sequences

| SEQ | | | |
|---|---|---|---|
| ID | Accession | | |
| NO: | No. | Name | SEQUENCE |
| 3 | YP_009742608 | Leader | MESLVPGFNEKTHVQLSLPVLQVRDVLVRG |
| | | protein | FGDSVEEVLSEARQHLKDGTCGLVEVEKGV |
| | | [Severe acute | LPQLEQPYVFIKRSDARTAPHGHVMVELVA |
| | | respiratory | ELEGIQYGRSGETLGVLVPHVGEIPVAYRK |
| | | syndrome | VLLRKNGNKGAGGHSYGADLKSFDLGDELG |
| | | coronavirus 2] | TDPYEDFQENWNTKHSSGVTRELMRELNGG |
| 4 | YP_009742609 | nsp2 [Severe | AYTRYVDNNFCGPDGYPLECIKDLLARAGK |
| | | acute | ASCTLSEQLDFIDTKRGVYCCREHEHEIAW |
| | | respiratory | YTERSEKSYELQTPFEIKLAKKFDTFNGEC |
| | | syndrome | PNFVFPLNSIIKTIQPRVEKKKLDGFMGRI |
| | | coronavirus 2] | RSVYPVASPNECNQMCLSTLMKCDHCGETS |
| | | | WQTGDFVKATCEFCGTENLTKEGATTCGYL |
| | | | PQNAVVKIYCPACHNSEVGPEHSLAEYHNE |
| | | | SGLKTILRKGGRTIAFGGCVFSYVGCHNKC |
| | | | AYWVPRASANIGCNHTGVVGEGSEGLNDNL |
| | | | LEILQKEKVNINIVGDFKLNEEIAIILASF |
| | | | SASTSAFVETVKGLDYKAFKQIVESCGNFK |
| | | | VTKGKAKKGAWNIGEQKSILSPLYAFASEA |
| | | | ARVVRSIFSRTLETAQNSVRVLQKAAITIL |
| | | | DGISQYSLRLIDAMMFTSDLATNNLVVMAY |
| | | | ITGGVVQLTSQWLTNIFGTVYEKLKPVLDW |
| | | | LEEKFKEGVEFLRDGWEIVKFISTCACEIV |
| | | | GGQIVTCAKEIKESVQTFFKLVNKFLALCA |
| | | | DSIIIGGAKLKALNLGETFVTHSKGLYRKC |
| | | | VKSREETGLLMPLKAPKEIIFLEGETLPTE |
| | | | VLTEEVVLKTGDLQPLEQPTSEAVEAPLVG |
| | | | TPVCINGLMLLEIKDTEKYCALAPNMMVTN |
| | | | NTFTLKGG |
| 5 | YP_009742610 | nsp3 | APTKVTFGDDTVIEVQGYKSVNITFELDER |
| | | [Severe | IDKVLNEKCSAYTVELGTEVNEFACVVADA |
| | | acute | VIKTLQPVSELLTPLGIDLDEWSMATYYLF |
| | | respiratory | DESGEFKLASHMYCSFYPPDEDEEEGDCEE |
| | | syndrome | EEFEPSTQYEYGTEDDYQGKPLEFGATSAA |
| | | coronavirus 2] | LQPEEEQEEDWLDDDSQQTVGQQDGSEDNQ |
| | | | TTTIQTIVEVQPQLEMELTPVVQTIEVNSF |
| | | | SGYLKLTDNVYIKNADIVEEAKKVKPTVVV |
| | | | NAANVYLKHGGGVAGALNKATNNAMQVESD |
| | | | DYIATNGPLKVGGSCVLSGHNLAKHCLHVV |
| | | | GPNVNKGEDIQLLKSAYENFNQHEVLLAPL |
| | | | LSAGIFGADPIHSLRVCVDTVRTNVYLAVF |
| | | | DKNLYDKLVSSFLEMKSEKQVEQKIAEIPK |
| | | | EEVKPFITESKPSVEQRKQDDKKIKACVEE |
| | | | VTTTLEETKFLTENLLLYIDINGNLHPDSA |
| | | | TLVSDIDITFLKKDAPYIVGDVVQEGVLTA |
| | | | VVIPTKKAGGTTEMLAKALRKVPTDNYITT |
| | | | YPGQGLNGYTVEEAKTVLKKCKSAFYILPS |
| | | | IISNEKQEILGTVSWNLREMLAHAEETRKL |
| | | | MPVCVETKAIVSTIQRKYKGIKIQEGVVDY |
| | | | GARFYFYTSKTTVASLINTLNDLNETLVTM |
| | | | PLGYVTHGLNLEEAARYMRSLKVPATVSVS |
| | | | SPDAVTAYNGYLTSSSKTPEEHFIETISLA |
| | | | GSYKDWSYSGQSTQLGIEFLKRGDKSVYYT |
| | | | SNPTTFHLDGEVITFDNLKTLLSLREVRTI |
| | | | KVFTTVDNINLHTQVVDMSMTYGQQFGPTY |
| | | | LDGADVTKIKPHNSHEGKTFYVLPNDDTLR |
| | | | VEAFEYYHTTDPSFLGRYMSALNHTKKWKY |
| | | | PQVNGLTSIKWADNNCYLATALLTLQQIEL |
| | | | KFNPPALQDAYYRARAGEAANFCALILAYC |
| | | | NKTVGELGDVRETMSYLFQHANLDSCKRVL |
| | | | NVVCKTCGQQQTTLKGVEAVMYMGTLSYEQ |
| | | | FKKGVQIPCTCGKQATKYLVQQESPFVMMS |
| | | | APPAQYELKHGTFTCASEYTGNYQCGHYKH |
| | | | ITSKETLYCIDGALLTKSSEYKGPITDVFY |
| | | | KENSYTTTIKPVTYKLDGVVCTEIDPKLDN |
| | | | YYKKDNSYFTEQPIDLVPNQPYPNASFDNF |
| | | | KFVCDNIKFADDLNQLTGYKKPASRELKVT |
| | | | FFPDLNGDVVAIDYKHYTPSFKKGAKLLHK |
| | | | PIVWHVNNATNKATYKPNTWCIRCLWSTKP |
| | | | VETSNSFDVLKSEDAQGMDNLACEDLKPVS |
| | | | EEVVENPTIQKDVLECNVKTTEVVGDIILK |
| | | | PANNSLKITEEVGHTDLMAAYVDNSSLTIK |
| | | | KPNELSRVLGLKTLATHGLAAVNSVPWDTI |
| | | | ANYAKPFLNKVVSTTTNIVTRCLNRVCTNY |
| | | | MPYFFTLLLQLCTFTRSTNSRIKASMPTTI |
| | | | AKNTVKSVGKFCLEASFNYLKSPNFSKLIN |
| | | | IIIWFLLLSVCLGSLIYSTAALGVLMSNLG |
| | | | MPSYCTGYREGYLNSTNVTIATYCTGSIPC |
| | | | SVCLSGLDSLDTYPSLETIQITISSFKWDL |
| | | | TAFGLVAEWFLAYILFTRFFYVLGLAAIMQ |
| | | | LFFSYFAVHFISNSWLMWLIINLVQMAPIS |
| | | | AMVRMYIFFASFYYVWKSYVHVVDGCNSST |
| | | | CMMCYKRNRATRVECTTIVNGVRRSFYVYA |
| | | | NGGKGFCKLHNWNCVNCDTFCAGSTFISDE |
| | | | VARDLSLQFKRPINPTDQSSYIVDSVTVKN |
| | | | GSIHLYFDKAGQKTYERHSLSHFVNLDNLR |
| | | | ANNTKGSLPINVIVFDGKSKCEESSAKSAS |
| | | | VYYSQLMCQPILLLDQALVSDVGDSAEVAV |
| | | | KMFDAYVNTFSSTFNVPMEKLKTLVATAEA |
| | | | ELAKNVSLDNVLSTFISAARQGFVDSDVET |
| | | | KDVVECLKLSHQSDIEVTGDSCNNYMLTYN |
| | | | KVENMTPRDLGACIDCSARHINAQVAKSHN |
| | | | IALIWNVKDFMSLSEQLRKQIRSAAKKNNL |
| | | | PFKLTCATTRQVVNVVTTKIALKGG |
| 6 | YP_009742611 | nsp4 | KIVNNWLKQLIKVTLVFLFVAAIFYLITPV |
| | | [Severe | HVMSKHTDFSSEIIGYKAIDGGVTRDIAST |
| | | acute | DTCFANKHADFDTWFSQRGGSYTNDKACPL |
| | | respiratory | IAAVITREVGFVVPGLPGTILRTTNGDFLH |
| | | syndrome | FLPRVFSAVGNICYTPSKLIEYTDFATSAC |
| | | coronavirus 2] | VLAAECTIFKDASGKPVPYCYDTNVLEGSV |
| | | | AYESLRPDTRYVLMDGSIIQFPNTYLEGSV |
| | | | RVVTTFDSEYCRHGTCERSEAGVCVSTSGR |
| | | | WVLNNDYYRSLPGVFCGVDAVNLLTNMFTP |
| | | | LIQPIGALDISASIVAGGIVAIVVTCLAYY |
| | | | FMRFRRAFGEYSHVVAFNTLLFLMSFTVLC |
| | | | LTPVYSFLPGVYSVIYLYLTFYLTNDVSFL |
| | | | AHIQWMVMFTPLVPFWITIAYIICISTKHF |
| | | | YWFFSNYLKRRVVFNGVSFSTFEEAALCTF |
| | | | LLNKEMYLKLRSDVLLPLTQYNRYLALYNK |
| | | | YKYFSGAMDTTSYREAACCHLAKALNDFSN |
| | | | SGSDVLYQPPQTSITSAVLQ |
| 7 | YP_009742612 | 3C-like | SGFRKMAFPSGKVEGCMVQVTCGTTTLNGL |
| | | proteinase | WLDDVVYCPRHVICTSEDMLNPNYEDLLIR |
| | | [Severe acute | KSNHNFLVQAGNVQLRVIGHSMQNCVLKLK |
| | | respiratory | VDTANPKTPKYKFVRIQPGQTFSVLACYNG |
| | | syndrome | SPSGVYQCAMRPNFTIKGSFLNGSCGSVGF |
| | | coronavirus 2] | NIDYDCVSFCYMHHMELPTGVHAGTDLEGN |
| | | | FYGPFVDRQTAQAAGTDTTITVNVLAWLYA |
| | | | AVINGDRWFLNRFTTTLNDFNLVAMKYNYE |
| | | | PLTQDHVDILGPLSAQTGIAVLDMCASLKE |
| | | | LLQNGMNGRTILGSALLEDEFTPFDVVRQC |
| | | | SGVTFQ |
| 8 | YP_009742613 | nsp6 [Severe | SAVKRTIKGTHHWLLLTILTSLLVLVQSTQ |
| | | acute | WSLFFFLYENAFLPFAMGIIAMSAFAMMFV |
| | | respiratory | KHKHAFLCLFLLPSLATVAYFNMVYMPASW |
| | | syndrome | VMRIMTWLDMVDTSLSGFKLKDCVMYASAV |
| | | coronavirus 2] | VLLILMTARTVYDDGARRVWTLMNVLTLVY |
| | | | KVYYGNALDQAISMWALIISVTSNYSGVVT |
| | | | TVMFLARGIVFMCVEYCPIFFITGNTLQCI |
| | | | MLVYCFLGYFCTCYFGLFCLLNRYFRLTLG |
| | | | VYDYLVSTQEFRYMNSQGLLPPKNSIDAFK |
| | | | LNIKLLGVGGKPCIKVATVQ |
| 9 | YP_009742614 | nsp7 [Severe | SKMSDVKCTSVVLLSVLQQLRVESSSKLWA |
| | | acute | QCVQLHNDILLAKDTTEAFEKMVSLLSVLL |
| | | respiratory | SMQGAVDINKLCEEMLDNRATLQ |
| | | syndrome | |
| | | coronavirus 2] | |
| 10 | YP_009742615 | nsp8 [Severe | AIASEFSSLPSYAAFATAQEAYEQAVANGD |
| | | acute | SEVVLKKLKKSLNVAKSEFDRDAAMQRKLE |
| | | respiratory | KMADQAMTQMYKQARSEDKRAKVTSAMQTM |
| | | syndrome | LFTMLRKLDNDALNNIINNARDGCVPLNII |
| | | coronavirus 2] | PLTTAAKLMVVIPDYNTYKNTCDGTTFTYA |
| | | | SALWEIQQVVDADSKIVQLSEISMDNSPNL |
| | | | AWPLIVTALRANSAVKLQ |
| 11 | YP_009742616 | nsp9 [Severe | NNELSPVALRQMSCAAGTTQTACTDDNALA |
| | | acute | YYNTTKGGRFVLALLSDLQDLKWARFPKSD |
| | | respiratory | GTGTIYTELEPPCRFVTDTPKGPKVKYLYF |
| | | syndrome | IKGLNNLNRGMVLGSLAATVRLQ |
| | | coronavirus 2] | |
| 12 | YP_009742617 | nsp10 [Severe | AGNATEVPANSTVLSFCAFAVDAAKAYKDY |
| | | acute | LASGGQPITNCVKMLCTHTGTGQAITVTPE |
| | | respiratory | ANMDQESFGGASCCLYCRCHIDHPNPKGFC |
| | | syndrome | DLKGKYVQIPTTCANDPVGFTLKNTVCTVC |
| | | coronavirus 2] | GMWKGYGCSCDQLREPMLQ |
| 13 | YP_009725318 | ORF7b [Severe | MIELSLIDFYLCFLAFLLFLVLIMLIIFWF |
| | | acute | SLELQDHNETCHA |
| | | respiratory | |
| | | syndrome | |
| | | coronavirus 2] | |
| 14 | YP_009725295 | orfla | MESLVPGFNEKTHVQLSLPVLQVRDVLVRG |
| | | polyprotein | FGDSVEEVLSEARQHLKDGTCGLVEVEKGV |
| | | [Severe acute | LPQLEQPYVFIKRSDARTAPHGHVMVELVA |
| | | respiratory | ELEGIQYGRSGETLGVLVPHVGEIPVAYRK |
| | | syndrome | VLLRKNGNKGAGGHSYGADLKSFDLGDELG |
| | | coronavirus 2] | TDPYEDFQENWNTKHSSGVTRELMRELNGG |
| | | | AYTRYVDNNFCGPDGYPLECIKDLLARAGK |
| | | | ASCTLSEQLDFIDTKRGVYCCREHEHEIAW |
| | | | YTERSEKSYELQTPFEIKLAKKFDTFNGEC |
| | | | PNFVFPLNSIIKTIQPRVEKKKLDGFMGRI |
| | | | RSVYPVASPNECNQMCLSTLMKCDHCGETS |
| | | | WQTGDFVKATCEFCGTENLTKEGATTCGYL |
| | | | PQNAVVKIYCPACHNSEVGPEHSLAEYHNE |
| | | | SGLKTILRKGGRTIAFGGCVFSYVGCHNKC |
| | | | AYWVPRASANIGCNHTGVVGEGSEGLNDNL |
| | | | LEILQKEKVNINIVGDFKLNEEIAIILASF |
| | | | SASTSAFVETVKGLDYKAFKQIVESCGNFK |
| | | | VTKGKAKKGAWNIGEQKSILSPLYAFASEA |
| | | | ARVVRSIFSRTLETAQNSVRVLQKAAITIL |
| | | | DGISQYSLRLIDAMMFTSDLATNNLVVMAY |
| | | | ITGGVVQLTSQWLTNIFGTVYEKLKPVLDW |
| | | | LEEKFKEGVEFLRDGWEIVKFISTCACEIV |
| | | | GGQIVTCAKEIKESVQTFFKLVNKFLALCA |
| | | | DSIIIGGAKLKALNLGETFVTHSKGLYRKC |
| | | | VKSREETGLLMPLKAPKEIIFLEGETLPTE |
| | | | VLTEEVVLKTGDLQPLEQPTSEAVEAPLVG |
| | | | TPVCINGLMLLEIKDTEKYCALAPNMMVTN |
| | | | NTFTLKGGAPTKVTFGDDTVIEVQGYKSVN |
| | | | ITFELDERIDKVLNEKCSAYTVELGTEVNE |
| | | | FACVVADAVIKTLQPVSELLTPLGIDLDEW |
| | | | SMATYYLFDESGEFKLASHMYCSFYPPDED |
| | | | EEEGDCEEEEFEPSTQYEYGTEDDYQGKPL |
| | | | EFGATSAALQPEEEQEEDWLDDDSQQTVGQ |
| | | | QDGSEDNQTTTIQTIVEVQPQLEMELTPVV |
| | | | QTIEVNSFSGYLKLTDNVYIKNADIVEEAK |
| | | | KVKPTVVVNAANVYLKHGGGVAGALNKATN |
| | | | NAMQVESDDYIATNGPLKVGGSCVLSGHNL |
| | | | AKHCLHVVGPNVNKGEDIQLLKSAYENFNQ |
| | | | HEVLLAPLLSAGIFGADPIHSLRVCVDTVR |
| | | | TNVYLAVFDKNLYDKLVSSFLEMKSEKQVE |
| | | | QKIAEIPKEEVKPFITESKPSVEQRKQDDK |
| | | | KIKACVEEVTTTLEETKFLTENLLLYIDIN |
| | | | GNLHPDSATLVSDIDITFLKKDAPYIVGDV |
| | | | VQEGVLTAVVIPTKKAGGTTEMLAKALRKV |
| | | | PTDNYITTYPGQGLNGYTVEEAKTVLKKCK |
| | | | SAFYILPSIISNEKQEILGTVSWNLREMLA |
| | | | HAEETRKLMPVCVETKAIVSTIQRKYKGIK |
| | | | IQEGVVDYGARFYFYTSKTTVASLINTLND |
| | | | LNETLVTMPLGYVTHGLNLEEAARYMRSLK |
| | | | VPATVSVSSPDAVTAYNGYLTSSSKTPEEH |
| | | | FIETISLAGSYKDWSYSGQSTQLGIEFLKR |
| | | | GDKSVYYTSNPTTFHLDGEVITFDNLKTLL |
| | | | SLREVRTIKVFTTVDNINLHTQVVDMSMTY |
| | | | GQQFGPTYLDGADVTKIKPHNSHEGKTFYV |
| | | | LPNDDTLRVEAFEYYHTTDPSFLGRYMSAL |
| | | | NHTKKWKYPQVNGLTSIKWADNNCYLATAL |
| | | | LTLQQIELKFNPPALQDAYYRARAGEAANF |
| | | | CALILAYCNKTVGELGDVRETMSYLFQHAN |
| | | | LDSCKRVLNVVCKTCGQQQTTLKGVEAVMY |
| | | | MGTLSYEQFKKGVQIPCTCGKQATKYLVQQ |
| | | | ESPFVMMSAPPAQYELKHGTFTCASEYTGN |
| | | | YQCGHYKHITSKETLYCIDGALLTKSSEYK |
| | | | GPITDVFYKENSYTTTIKPVTYKLDGVVCT |
| | | | EIDPKLDNYYKKDNSYFTEQPIDLVPNQPY |
| | | | PNASFDNFKFVCDNIKFADDLNQLTGYKKP |
| | | | ASRELKVTFFPDLNGDVVAIDYKHYTPSFK |
| | | | KGAKLLHKPIVWHVNNATNKATYKPNTWCI |
| | | | RCLWSTKPVETSNSFDVLKSEDAQGMDNLA |
| | | | CEDLKPVSEEVVENPTIQKDVLECNVKTTE |
| | | | VVGDIILKPANNSLKITEEVGHTDLMAAYV |
| | | | DNSSLTIKKPNELSRVLGLKTLATHGLAAV |
| | | | NSVPWDTIANYAKPFLNKVVSTTTNIVTRC |
| | | | LNRVCTNYMPYFFTLLLQLCTFTRSTNSRI |
| | | | KASMPTTIAKNTVKSVGKFCLEASFNYLKS |
| | | | PNFSKLINIIIWFLLLSVCLGSLIYSTAAL |
| | | | GVLMSNLGMPSYCTGYREGYLNSTNVTIAT |
| | | | YCTGSIPCSVCLSGLDSLDTYPSLETIQIT |
| | | | ISSFKWDLTAFGLVAEWFLAYILFTRFFYV |
| | | | LGLAAIMQLFFSYFAVHFISNSWLMWLIIN |
| | | | LVQMAPISAMVRMYIFFASFYYVWKSYVHV |
| | | | VDGCNSSTCMMCYKRNRATRVECTTIVNGV |
| | | | RRSFYVYANGGKGFCKLHNWNCVNCDTFCA |
| | | | GSTFISDEVARDLSLQFKRPINPTDQSSYI |
| | | | VDSVTVKNGSIHLYFDKAGQKTYERHSLSH |
| | | | FVNLDNLRANNTKGSLPINVIVFDGKSKCE |
| | | | ESSAKSASVYYSQLMCQPILLLDQALVSDV |
| | | | GDSAEVAVKMFDAYVNTFSSTFNVPMEKLK |
| | | | TLVATAEAELAKNVSLDNVLSTFISAARQG |
| | | | FVDSDVETKDVVECLKLSHQSDIEVTGDSC |
| | | | NNYMLTYNKVENMTPRDLGACIDCSARHIN |
| | | | AQVAKSHNIALIWNVKDFMSLSEQLRKQIR |
| | | | SAAKKNNLPFKLTCATTRQVVNVVTTKIAL |
| | | | KGGKIVNNWLKQLIKVTLVFLFVAAIFYLI |
| | | | TPVHVMSKHTDFSSEIIGYKAIDGGVTRDI |
| | | | ASTDTCFANKHADFDTWFSQRGGSYTNDKA |
| | | | CPLIAAVITREVGFVVPGLPGTILRTTNGD |
| | | | FLHFLPRVFSAVGNICYTPSKLIEYTDFAT |
| | | | SACVLAAECTIFKDASGKPVPYCYDTNVLE |
| | | | GSVAYESLRPDTRYVLMDGSIIQFPNTYLE |
| | | | GSVRVVTTFDSEYCRHGTCERSEAGVCVST |
| | | | SGRWVLNNDYYRSLPGVFCGVDAVNLLTNM |
| | | | FTPLIQPIGALDISASIVAGGIVAIVVTCL |
| | | | AYYFMRFRRAFGEYSHVVAFNTLLFLMSFT |
| | | | VLCLTPVYSFLPGVYSVIYLYLTFYLTNDV |
| | | | SFLAHIQWMVMFTPLVPFWITIAYIICIST |
| | | | KHFYWFFSNYLKRRVVFNGVSFSTFEEAAL |
| | | | CTFLLNKEMYLKLRSDVLLPLTQYNRYLAL |
| | | | YNKYKYFSGAMDTTSYREAACCHLAKALND |
| | | | FSNSGSDVLYQPPQTSITSAVLQSGFRKMA |
| | | | FPSGKVEGCMVQVTCGTTTLNGLWLDDVVY |
| | | | CPRHVICTSEDMLNPNYEDLLIRKSNHNFL |
| | | | VQAGNVQLRVIGHSMQNCVLKLKVDTANPK |
| | | | TPKYKFVRIQPGQTFSVLACYNGSPSGVYQ |
| | | | CAMRPNFTIKGSFLNGSCGSVGFNIDYDCV |
| | | | SFCYMHHMELPTGVHAGTDLEGNFYGPFVD |
| | | | RQTAQAAGTDTTITVNVLAWLYAAVINGDR |
| | | | WFLNRFTTTLNDFNLVAMKYNYEPLTQDHV |
| | | | DILGPLSAQTGIAVLDMCASLKELLQNGMN |
| | | | GRTILGSALLEDEFTPFDVVRQCSGVTFQS |
| | | | AVKRTIKGTHHWLLLTILTSLLVLVQSTQW |
| | | | SLFFFLYENAFLPFAMGIIAMSAFAMMFVK |
| | | | HKHAFLCLFLLPSLATVAYFNMVYMPASWV |
| | | | MRIMTWLDMVDTSLSGFKLKDCVMYASAVV |
| | | | LLILMTARTVYDDGARRVWTLMNVLTLVYK |
| | | | VYYGNALDQAISMWALIISVTSNYSGVVTT |
| | | | VMFLARGIVFMCVEYCPIFFITGNTLQCIM |
| | | | LVYCFLGYFCTCYFGLFCLLNRYFRLTLGV |
| | | | YDYLVSTQEFRYMNSQGLLPPKNSIDAFKL |
| | | | NIKLLGVGGKPCIKVATVQSKMSDVKCTSV |
| | | | VLLSVLQQLRVESSSKLWAQCVQLHNDILL |
| | | | AKDTTEAFEKMVSLLSVLLSMQGAVDINKL |
| | | | CEEMLDNRATLQAIASEFSSLPSYAAFATA |
| | | | QEAYEQAVANGDSEVVLKKLKKSLNVAKSE |
| | | | FDRDAAMQRKLEKMADQAMTQMYKQARSED |
| | | | KRAKVTSAMQTMLFTMLRKLDNDALNNIIN |
| | | | NARDGCVPLNIIPLTTAAKLMVVIPDYNTY |
| | | | KNTCDGTTFTYASALWEIQQVVDADSKIVQ |
| | | | LSEISMDNSPNLAWPLIVTALRANSAVKLQ |
| | | | NNELSPVALRQMSCAAGTTQTACTDDNALA |
| | | | YYNTTKGGRFVLALLSDLQDLKWARFPKSD |
| | | | GTGTIYTELEPPCRFVTDTPKGPKVKYLYF |
| | | | IKGLNNLNRGMVLGSLAATVRLQAGNATEV |
| | | | PANSTVLSFCAFAVDAAKAYKDYLASGGQP |
| | | | ITNCVKMLCTHTGTGQAITVTPEANMDQES |
| | | | FGGASCCLYCRCHIDHPNPKGFCDLKGKYV |
| | | | QIPTTCANDPVGFTLKNTVCTVCGMWKGYG |
| | | | CSCDQLREPMLQSADAQSFLNGFAV |
| 15 | YP_009725297 | leader protein | MESLVPGFNEKTHVQLSLPVLQVRDVLVRG |
| | | [Severe acute | FGDSVEEVLSEARQHLKDGTCGLVEVEKGV |
| | | respiratory | LPQLEQPYVFIKRSDARTAPHGHVMVELVA |
| | | syndrome | ELEGIQYGRSGETLGVLVPHVGEIPVAYRK |
| | | coronavirus 2] | VLLRKNGNKGAGGHSYGADLKSFDLGDELG |
| | | | TDPYEDFQENWNTKHSSGVTRELMRELNGG |
| 16 | YP_009725298 | nsp2 [Severe | AYTRYVDNNFCGPDGYPLECIKDLLARAGK |
| | | acute | ASCTLSEQLDFIDTKRGVYCCREHEHEIAW |
| | | respiratory | YTERSEKSYELQTPFEIKLAKKFDTFNGEC |
| | | syndrome | PNFVFPLNSIIKTIQPRVEKKKLDGFMGRI |
| | | coronavirus 2] | RSVYPVASPNECNQMCLSTLMKCDHCGETS |
| | | | WQTGDFVKATCEFCGTENLTKEGATTCGYL |
| | | | PQNAVVKIYCPACHNSEVGPEHSLAEYHNE |
| | | | SGLKTILRKGGRTIAFGGCVFSYVGCHNKC |
| | | | AYWVPRASANIGCNHTGVVGEGSEGLNDNL |
| | | | LEILQKEKVNINIVGDFKLNEEIAIILASF |
| | | | SASTSAFVETVKGLDYKAFKQIVESCGNFK |
| | | | VTKGKAKKGAWNIGEQKSILSPLYAFASEA |
| | | | ARVVRSIFSRTLETAQNSVRVLQKAAITIL |
| | | | DGISQYSLRLIDAMMFTSDLATNNLVVMAY |
| | | | ITGGVVQLTSQWLTNIFGTVYEKLKPVLDW |
| | | | LEEKFKEGVEFLRDGWEIVKFISTCACEIV |
| | | | GGQIVTCAKEIKESVQTFFKLVNKFLALCA |
| | | | DSIIIGGAKLKALNLGETFVTHSKGLYRKC |
| | | | VKSREETGLLMPLKAPKEIIFLEGETLPTE |
| | | | VLTEEVVLKTGDLQPLEQPTSEAVEAPLVG |
| | | | TPVCINGLMLLEIKDTEKYCALAPNMMVTN |
| | | | NTFTLKGG |
| 17 | YP_009725299 | nsp3 [Severe | APTKVTFGDDTVIEVQGYKSVNITFELDER |
| | | acute | IDKVLNEKCSAYTVELGTEVNEFACVVADA |
| | | respiratory | VIKTLQPVSELLTPLGIDLDEWSMATYYLF |
| | | syndrome | DESGEFKLASHMYCSFYPPDEDEEEGDCEE |
| | | coronavirus 2] | EEFEPSTQYEYGTEDDYQGKPLEFGATSAA |
| | | | LQPEEEQEEDWLDDDSQQTVGQQDGSEDNQ |
| | | | TTTIQTIVEVQPQLEMELTPVVQTIEVNSF |
| | | | SGYLKLTDNVYIKNADIVEEAKKVKPTVVV |
| | | | NAANVYLKHGGGVAGALNKATNNAMQVESD |
| | | | DYIATNGPLKVGGSCVLSGHNLAKHCLHVV |
| | | | GPNVNKGEDIQLLKSAYENFNQHEVLLAPL |
| | | | LSAGIFGADPIHSLRVCVDTVRTNVYLAVF |
| | | | DKNLYDKLVSSFLEMKSEKQVEQKIAEIPK |
| | | | EEVKPFITESKPSVEQRKQDDKKIKACVEE |
| | | | VTTTLEETKFLTENLLLYIDINGNLHPDSA |
| | | | TLVSDIDITFLKKDAPYIVGDVVQEGVLTA |
| | | | VVIPTKKAGGTTEMLAKALRKVPTDNYITT |
| | | | YPGQGLNGYTVEEAKTVLKKCKSAFYILPS |
| | | | IISNEKQEILGTVSWNLREMLAHAEETRKL |
| | | | MPVCVETKAIVSTIQRKYKGIKIQEGVVDY |
| | | | GARFYFYTSKTTVASLINTLNDLNETLVTM |
| | | | PLGYVTHGLNLEEAARYMRSLKVPATVSVS |
| | | | SPDAVTAYNGYLTSSSKTPEEHFIETISLA |
| | | | GSYKDWSYSGQSTQLGIEFLKRGDKSVYYT |
| | | | SNPTTFHLDGEVITFDNLKTLLSLREVRTI |
| | | | KVFTTVDNINLHTQVVDMSMTYGQQFGPTY |
| | | | LDGADVTKIKPHNSHEGKTFYVLPNDDTLR |
| | | | VEAFEYYHTTDPSFLGRYMSALNHTKKWKY |
| | | | PQVNGLTSIKWADNNCYLATALLTLQQIEL |
| | | | KFNPPALQDAYYRARAGEAANFCALILAYC |
| | | | NKTVGELGDVRETMSYLFQHANLDSCKRVL |
| | | | NVVCKTCGQQQTTLKGVEAVMYMGTLSYEQ |
| | | | FKKGVQIPCTCGKQATKYLVQQESPFVMMS |
| | | | APPAQYELKHGTFTCASEYTGNYQCGHYKH |
| | | | ITSKETLYCIDGALLTKSSEYKGPITDVFY |
| | | | KENSYTTTIKPVTYKLDGVVCTEIDPKLDN |
| | | | YYKKDNSYFTEQPIDLVPNQPYPNASFDNF |
| | | | KFVCDNIKFADDLNQLTGYKKPASRELKVT |
| | | | FFPDLNGDVVAIDYKHYTPSFKKGAKLLHK |
| | | | PIVWHVNNATNKATYKPNTWCIRCLWSTKP |
| | | | VETSNSFDVLKSEDAQGMDNLACEDLKPVS |
| | | | EEVVENPTIQKDVLECNVKTTEVVGDIILK |
| | | | PANNSLKITEEVGHTDLMAAYVDNSSLTIK |
| | | | KPNELSRVLGLKTLATHGLAAVNSVPWDTI |
| | | | ANYAKPFLNKVVSTTTNIVTRCLNRVCTNY |
| | | | MPYFFTLLLQLCTFTRSTNSRIKASMPTTI |
| | | | AKNTVKSVGKFCLEASFNYLKSPNFSKLIN |
| | | | IIIWFLLLSVCLGSLIYSTAALGVLMSNLG |
| | | | MPSYCTGYREGYLNSTNVTIATYCTGSIPC |
| | | | SVCLSGLDSLDTYPSLETIQITISSFKWDL |
| | | | TAFGLVAEWFLAYILFTRFFYVLGLAAIMQ |
| | | | LFFSYFAVHFISNSWLMWLIINLVQMAPIS |
| | | | AMVRMYIFFASFYYVWKSYVHVVDGCNSST |
| | | | CMMCYKRNRATRVECTTIVNGVRRSFYVYA |
| | | | NGGKGFCKLHNWNCVNCDTFCAGSTFISDE |
| | | | VARDLSLQFKRPINPTDQSSYIVDSVTVKN |
| | | | GSIHLYFDKAGQKTYERHSLSHFVNLDNLR |
| | | | ANNTKGSLPINVIVFDGKSKCEESSAKSAS |
| | | | VYYSQLMCQPILLLDQALVSDVGDSAEVAV |
| | | | KMFDAYVNTFSSTFNVPMEKLKTLVATAEA |
| | | | ELAKNVSLDNVLSTFISAARQGFVDSDVET |
| | | | KDVVECLKLSHQSDIEVTGDSCNNYMLTYN |
| | | | KVENMTPRDLGACIDCSARHINAQVAKSHN |
| | | | IALIWNVKDFMSLSEQLRKQIRSAAKKNNL |
| | | | PFKLTCATTRQVVNVVTTKIALKGG |
| 18 | YP_009725300 | nsp4 [Severe | KIVNNWLKQLIKVTLVFLFVAAIFYLITPV |
| | | acute | HVMSKHTDFSSEIIGYKAIDGGVTRDIAST |
| | | respiratory | DTCFANKHADFDTWFSQRGGSYTNDKACPL |
| | | syndrome | IAAVITREVGFVVPGLPGTILRTTNGDFLH |
| | | coronavirus 2] | FLPRVFSAVGNICYTPSKLIEYTDFATSAC |
| | | | VLAAECTIFKDASGKPVPYCYDTNVLEGSV |
| | | | AYESLRPDTRYVLMDGSIIQFPNTYLEGSV |
| | | | RVVTTFDSEYCRHGTCERSEAGVCVSTSGR |
| | | | WVLNNDYYRSLPGVFCGVDAVNLLTNMFTP |
| | | | LIQPIGALDISASIVAGGIVAIVVTCLAYY |
| | | | FMRFRRAFGEYSHVVAFNTLLFLMSFTVLC |
| | | | LTPVYSFLPGVYSVIYLYLTFYLTNDVSFL |
| | | | AHIQWMVMFTPLVPFWITIAYIICISTKHF |
| | | | YWFFSNYLKRRVVFNGVSFSTFEEAALCTF |
| | | | LLNKEMYLKLRSDVLLPLTQYNRYLALYNK |
| | | | YKYFSGAMDTTSYREAACCHLAKALNDFSN |
| | | | SGSDVLYQPPQTSITSAVLQ |
| 19 | YP_009725301 | 3C-like | SGFRKMAFPSGKVEGCMVQVTCGTTTLNGL |
| | | proteinase | WLDDVVYCPRHVICTSEDMLNPNYEDLLIR |
| | | [Severe acute | KSNHNFLVQAGNVQLRVIGHSMQNCVLKLK |
| | | respiratory | VDTANPKTPKYKFVRIQPGQTFSVLACYNG |
| | | syndrome | SPSGVYQCAMRPNFTIKGSFLNGSCGSVGF |
| | | coronavirus 2] | NIDYDCVSFCYMHHMELPTGVHAGTDLEGN |
| | | | FYGPFVDRQTAQAAGTDTTITVNVLAWLYA |
| | | | AVINGDRWFLNRFTTTLNDFNLVAMKYNYE |
| | | | PLTQDHVDILGPLSAQTGIAVLDMCASLKE |
| | | | LLQNGMNGRTILGSALLEDEFTPFDVVRQC |
| | | | SGVTFQ |
| 20 | YP_009725302 | nsp6 [Severe | SAVKRTIKGTHHWLLLTILTSLLVLVQSTQ |
| | | acute | WSLFFFLYENAFLPFAMGIIAMSAFAMMFV |
| | | respiratory | KHKHAFLCLFLLPSLATVAYFNMVYMPASW |
| | | syndrome | VMRIMTWLDMVDTSLSGFKLKDCVMYASAV |
| | | coronavirus 2] | VLLILMTARTVYDDGARRVWTLMNVLTLVY |
| | | | KVYYGNALDQAISMWALIISVTSNYSGVVT |
| | | | TVMFLARGIVFMCVEYCPIFFITGNTLQCI |
| | | | MLVYCFLGYFCTCYFGLFCLLNRYFRLTLG |
| | | | VYDYLVSTQEFRYMNSQGLLPPKNSIDAFK |
| | | | LNIKLLGVGGKPCIKVATVQ |
| 21 | YP_009725303 | nsp7 [Severe | SKMSDVKCTSVVLLSVLQQLRVESSSKLWA |
| | | acute | QCVQLHNDILLAKDTTEAFEKMVSLLSVLL |
| | | respiratory | SMQGAVDINKLCEEMLDNRATLQ |
| | | syndrome | |
| | | coronavirus 2] | |
| 22 | YP_009725304 | nsp8 [Severe | AIASEFSSLPSYAAFATAQEAYEQAVANGD |
| | | acute | SEVVLKKLKKSLNVAKSEFDRDAAMQRKLE |
| | | respiratory | KMADQAMTQMYKQARSEDKRAKVTSAMQTM |
| | | syndrome | LFTMLRKLDNDALNNIINNARDGCVPLNII |
| | | coronavirus 2] | PLTTAAKLMVVIPDYNTYKNTCDGTTFTYA |
| | | | SALWEIQQVVDADSKIVQLSEISMDNSPNL |
| | | | AWPLIVTALRANSAVKLQ |
| 23 | YP_009725305 | nsp9 [Severe | NNELSPVALRQMSCAAGTTQTACTDDNALA |
| | | acute | YYNTTKGGRFVLALLSDLQDLKWARFPKSD |
| | | respiratory | GTGTIYTELEPPCRFVTDTPKGPKVKYLYF |
| | | syndrome | IKGLNNLNRGMVLGSLAATVRLQ |
| | | coronavirus 2] | |
| 24 | YP_009725306 | nsp10 [Severe | AGNATEVPANSTVLSFCAFAVDAAKAYKDY |
| | | acute | LASGGQPITNCVKMLCTHTGTGQAITVTPE |
| | | respiratory | ANMDQESFGGASCCLYCRCHIDHPNPKGFC |
| | | syndrome | DLKGKYVQIPTTCANDPVGFTLKNTVCTVC |
| | | coronavirus 2] | GMWKGYGCSCDQLREPMLQ |
| 25 | YP_009725307 | RNA-dependent | SADAQSFLNRVCGVSAARLTPCGTGTSTDV |
| | | RNA polymerase | VYRAFDIYNDKVAGFAKFLKTNCCRFQEKD |
| | | [Severe acute | EDDNLIDSYFVVKRHTFSNYQHEETIYNLL |
| | | respiratory | KDCPAVAKHDFFKFRIDGDMVPHISRQRLT |
| | | syndrome | KYTMADLVYALRHFDEGNCDTLKEILVTYN |
| | | coronavirus 2] | CCDDDYFNKKDWYDFVENPDILRVYANLGE |
| | | | RVRQALLKTVQFCDAMRNAGIVGVLTLDNQ |
| | | | DLNGNWYDFGDFIQTTPGSGVPVVDSYYSL |
| | | | LMPILTLTRALTAESHVDTDLTKPYIKWDL |
| | | | LKYDFTEERLKLFDRYFKYWDQTYHPNCVN |
| | | | CLDDRCILHCANFNVLFSTVFPPTSFGPLV |
| | | | RKIFVDGVPFVVSTGYHFRELGVVHNQDVN |
| | | | LHSSRLSFKELLVYAADPAMHAASGNLLLD |
| | | | KRTTCFSVAALTNNVAFQTVKPGNFNKDFY |
| | | | DFAVSKGFFKEGSSVELKHFFFAQDGNAAI |
| | | | SDYDYYRYNLPTMCDIRQLLFVVEVVDKYF |
| | | | DCYDGGCINANQVIVNNLDKSAGFPFNKWG |
| | | | KARLYYDSMSYEDQDALFAYTKRNVIPTIT |
| | | | QMNLKYAISAKNRARTVAGVSICSTMTNRQ |
| | | | FHQKLLKSIAATRGATVVIGTSKFYGGWHN |
| | | | MLKTVYSDVENPHLMGWDYPKCDRAMPNML |
| | | | RIMASLVLARKHTTCCSLSHRFYRLANECA |
| | | | QVLSEMVMCGGSLYVKPGGTSSGDATTAYA |
| | | | NSVFNICQAVTANVNALLSTDGNKIADKYV |
| | | | RNLQHRLYECLYRNRDVDTDFVNEFYAYLR |
| | | | KHFSMMILSDDAVVCFNSTYASQGLVASIK |
| | | | NFKSVLYYQNNVFMSEAKCWTETDLTKGPH |
| | | | EFCSQHTMLVKQGDDYVYLPYPDPSRILGA |
| | | | GCFVDDIVKTDGTLMIERFVSLAIDAYPLT |
| | | | KHPNQEYADVFHLYLQYIRKLHDELTGHML |
| | | | DMYSVMLTNDNTSRYWEPEFYEAMYTPHTV |
| | | | LQ |
| 26 | YP_009725308 | helicase [Severe | AVGACVLCNSQTSLRCGACIRRPFLCCKCC |
| | | acute | YDHVISTSHKLVLSVNPYVCNAPGCDVTDV |
| | | respiratory | TQLYLGGMSYYCKSHKPPISFPLCANGQVF |
| | | syndrome | GLYKNTCVGSDNVTDFNAIATCDWTNAGDY |
| | | coronavirus 2] | ILANTCTERLKLFAAETLKATEETFKLSYG |
| | | | IATVREVLSDRELHLSWEVGKPRPPLNRNY |
| | | | VFTGYRVTKNSKVQIGEYTFEKGDYGDAVV |
| | | | YRGTTTYKLNVGDYFVLTSHTVMPLSAPTL |
| | | | VPQEHYVRITGLYPTLNISDEFSSNVANYQ |
| | | | KVGMQKYSTLQGPPGTGKSHFAIGLALYYP |
| | | | SARIVYTACSHAAVDALCEKALKYLPIDKC |
| | | | SRIIPARARVECFDKFKVNSTLEQYVFCTV |
| | | | NALPETTADIVVFDEISMATNYDLSVVNAR |
| | | | LRAKHYVYIGDPAQLPAPRTLLTKGTLEPE |
| | | | YFNSVCRLMKTIGPDMFLGTCRRCPAEIVD |
| | | | TVSALVYDNKLKAHKDKSAQCFKMFYKGVI |
| | | | THDVSSAINRPQIGVVREFLTRNPAWRKAV |
| | | | FISPYNSQNAVASKILGLPTQTVDSSQGSE |
| | | | YDYVIFTQTTETAHSCNVNRFNVAITRAKV |
| | | | GILCIMSDRDLYDKLQFTSLEIPRRNVATL |
| | | | Q |
| 27 | YP_009725309 | 3′-to-5′ | AENVTGLFKDCSKVITGLHPTQAPTHLSVD |
| | | exonuclease | TKFKTEGLCVDIPGIPKDMTYRRLISMMGF |
| | | [Severe acute | KMNYQVNGYPNMFITREEAIRHVRAWIGFD |
| | | respiratory | VEGCHATREAVGTNLPLQLGFSTGVNLVAV |
| | | syndrome | PTGYVDTPNNTDFSRVSAKPPPGDQFKHLI |
| | | coronavirus 2] | PLMYKGLPWNVVRIKIVQMLSDTLKNLSDR |
| | | | VVFVLWAHGFELTSMKYFVKIGPERTCCLC |
| | | | DRRATCFSTASDTYACWHHSIGFDYVYNPF |
| | | | MIDVQQWGFTGNLQSNHDLYCQVHGNAHVA |
| | | | SCDAIMTRCLAVHECFVKRVDWTIEYPIIG |
| | | | DELKINAACRKVQHMVVKAALLADKFPVLH |
| | | | DIGNPKAIKCVPQADVEWKFYDAQPCSDKA |
| | | | YKIEELFYSYATHSDKFTDGVCLFWNCNVD |
| | | | RYPANSIVCRFDTRVLSNLNLPGCDGGSLY |
| | | | VNKHAFHTPAFDKSAFVNLKQLPFFYYSDS |
| | | | PCESHGKQVVSDIDYVPLKSATCITRCNLG |
| | | | GAVCRHHANEYRLYLDAYNMMISAGFSLWV |
| | | | YKQFDTYNLWNTFTRLQ |
| 28 | YP_009725310 | endoRNAse | SLENVAFNVVNKGHFDGQQGEVPVSIINNT |
| | | [Severe acute | VYTKVDGVDVELFENKTTLPVNVAFELWAK |
| | | respiratory | RNIKPVPEVKILNNLGVDIAANTVIWDYKR |
| | | syndrome | DAPAHISTIGVCSMTDIAKKPTETICAPLT |
| | | coronavirus 2] | VFFDGRVDGQVDLFRNARNGVLITEGSVKG |
| | | | LQPSVGPKQASLNGVTLIGEAVKTQFNYYK |
| | | | KVDGVVQQLPETYFTQSRNLQEFKPRSQME |
| | | | IDFLELAMDEFIERYKLEGYAFEHIVYGDF |
| | | | SHSQLGGLHLLIGLAKRFKESPFELEDFIP |
| | | | MDSTVKNYFITDAQTGSSKCVCSVIDLLLD |
| | | | DFVEIIKSQDLSVVSKVVKVTIDYTEISFM |
| | | | LWCKDGHVETFYPKLQ |
| 29 | YP_009725311 | 2′-O-ribose | SSQAWQPGVAMPNLYKMQRMLLEKCDLQNY |
| | | methyltransferase | GDSATLPKGIMMNVAKYTQLCQYLNTLTLA |
| | | [Severe acute | VPYNMRVIHFGAGSDKGVAPGTAVLRQWLP |
| | | respiratory | TGTLLVDSDLNDFVSDADSTLIGDCATVHT |
| | | syndrome | ANKWDLIISDMYDPKTKNVTKENDSKEGFF |
| | | coronavirus 2] | TYICGFIQQKLALGGSVAIKITEHSWNADL |
| | | | YKLMGHFAWWTAFVTNVNASSSEAFLIGCN |
| | | | YLGKPREQIDGYVMHANYIFWRNTNPIQLS |
| | | | SYSLFDMSKFPLKLRGTAVMSLKEGQINDM |
| | | | ILSLLSKGRLIIRENNRVVISSDVLVNN |
| 30 | YP_009725312 | nsp11 [Severe | SADAQSFLNGFAV |
| | | acute | |
| | | respiratory | |
| | | syndrome | |
| | | coronavirus 2] | |
| 31 | YP_009725255 | ORF10 protein | MGYINVFAFPFTIYSLLLCRMNSRNYIAQV |
| | | [Severe acute | DVVNFNLT |
| | | respiratory | |
| | | syndrome | |
| | | coronavirus 2] | |
| 32 | YP_009724389 | orflab | MESLVPGFNEKTHVQLSLPVLQVRDVLVRG |
| | | polyprotein | FGDSVEEVLSEARQHLKDGTCGLVEVEKGV |
| | | [Severe acute | LPQLEQPYVFIKRSDARTAPHGHVMVELVA |
| | | respiratory | ELEGIQYGRSGETLGVLVPHVGEIPVAYRK |
| | | syndrome | VLLRKNGNKGAGGHSYGADLKSFDLGDELG |
| | | coronavirus 2] | TDPYEDFQENWNTKHSSGVTRELMRELNGG |
| | | | AYTRYVDNNFCGPDGYPLECIKDLLARAGK |
| | | | ASCTLSEQLDFIDTKRGVYCCREHEHEIAW |
| | | | YTERSEKSYELQTPFEIKLAKKFDTFNGEC |
| | | | PNFVFPLNSIIKTIQPRVEKKKLDGFMGRI |
| | | | RSVYPVASPNECNQMCLSTLMKCDHCGETS |
| | | | WQTGDFVKATCEFCGTENLTKEGATTCGYL |
| | | | PQNAVVKIYCPACHNSEVGPEHSLAEYHNE |
| | | | SGLKTILRKGGRTIAFGGCVFSYVGCHNKC |
| | | | AYWVPRASANIGCNHTGVVGEGSEGLNDNL |
| | | | LEILQKEKVNINIVGDFKLNEEIAIILASF |
| | | | SASTSAFVETVKGLDYKAFKQIVESCGNFK |
| | | | VTKGKAKKGAWNIGEQKSILSPLYAFASEA |
| | | | ARVVRSIFSRTLETAQNSVRVLQKAAITIL |
| | | | DGISQYSLRLIDAMMFTSDLATNNLVVMAY |
| | | | ITGGVVQLTSQWLTNIFGTVYEKLKPVLDW |
| | | | LEEKFKEGVEFLRDGWEIVKFISTCACEIV |
| | | | GGQIVTCAKEIKESVQTFFKLVNKFLALCA |
| | | | DSIIIGGAKLKALNLGETFVTHSKGLYRKC |
| | | | VKSREETGLLMPLKAPKEIIFLEGETLPTE |
| | | | VLTEEVVLKTGDLQPLEQPTSEAVEAPLVG |
| | | | TPVCINGLMLLEIKDTEKYCALAPNMMVTN |
| | | | NTFTLKGGAPTKVTFGDDTVIEVQGYKSVN |
| | | | ITFELDERIDKVLNEKCSAYTVELGTEVNE |
| | | | FACVVADAVIKTLQPVSELLTPLGIDLDEW |
| | | | SMATYYLFDESGEFKLASHMYCSFYPPDED |
| | | | EEEGDCEEEEFEPSTQYEYGTEDDYQGKPL |
| | | | EFGATSAALQPEEEQEEDWLDDDSQQTVGQ |
| | | | QDGSEDNQTTTIQTIVEVQPQLEMELTPVV |
| | | | QTIEVNSFSGYLKLTDNVYIKNADIVEEAK |
| | | | KVKPTVVVNAANVYLKHGGGVAGALNKATN |
| | | | NAMQVESDDYIATNGPLKVGGSCVLSGHNL |
| | | | AKHCLHVVGPNVNKGEDIQLLKSAYENFNQ |
| | | | HEVLLAPLLSAGIFGADPIHSLRVCVDTVR |
| | | | TNVYLAVFDKNLYDKLVSSFLEMKSEKQVE |
| | | | QKIAEIPKEEVKPFITESKPSVEQRKQDDK |
| | | | KIKACVEEVTTTLEETKFLTENLLLYIDIN |
| | | | GNLHPDSATLVSDIDITFLKKDAPYIVGDV |
| | | | VQEGVLTAVVIPTKKAGGTTEMLAKALRKV |
| | | | PTDNYITTYPGQGLNGYTVEEAKTVLKKCK |
| | | | SAFYILPSIISNEKQEILGTVSWNLREMLA |
| | | | HAEETRKLMPVCVETKAIVSTIQRKYKGIK |
| | | | IQEGVVDYGARFYFYTSKTTVASLINTLND |
| | | | LNETLVTMPLGYVTHGLNLEEAARYMRSLK |
| | | | VPATVSVSSPDAVTAYNGYLTSSSKTPEEH |
| | | | FIETISLAGSYKDWSYSGQSTQLGIEFLKR |
| | | | GDKSVYYTSNPTTFHLDGEVITFDNLKTLL |
| | | | SLREVRTIKVFTTVDNINLHTQVVDMSMTY |
| | | | GQQFGPTYLDGADVTKIKPHNSHEGKTFYV |
| | | | LPNDDTLRVEAFEYYHTTDPSFLGRYMSAL |
| | | | NHTKKWKYPQVNGLTSIKWADNNCYLATAL |
| | | | LTLQQIELKFNPPALQDAYYRARAGEAANF |
| | | | CALILAYCNKTVGELGDVRETMSYLFQHAN |
| | | | LDSCKRVLNVVCKTCGQQQTTLKGVEAVMY |
| | | | MGTLSYEQFKKGVQIPCTCGKQATKYLVQQ |
| | | | ESPFVMMSAPPAQYELKHGTFTCASEYTGN |
| | | | YQCGHYKHITSKETLYCIDGALLTKSSEYK |
| | | | GPITDVFYKENSYTTTIKPVTYKLDGVVCT |
| | | | EIDPKLDNYYKKDNSYFTEQPIDLVPNQPY |
| | | | PNASFDNFKFVCDNIKFADDLNQLTGYKKP |
| | | | ASRELKVTFFPDLNGDVVAIDYKHYTPSFK |
| | | | KGAKLLHKPIVWHVNNATNKATYKPNTWCI |
| | | | RCLWSTKPVETSNSFDVLKSEDAQGMDNLA |
| | | | CEDLKPVSEEVVENPTIQKDVLECNVKTTE |
| | | | VVGDIILKPANNSLKITEEVGHTDLMAAYV |
| | | | DNSSLTIKKPNELSRVLGLKTLATHGLAAV |
| | | | NSVPWDTIANYAKPFLNKVVSTTTNIVTRC |
| | | | LNRVCTNYMPYFFTLLLQLCTFTRSTNSRI |
| | | | KASMPTTIAKNTVKSVGKFCLEASFNYLKS |
| | | | PNFSKLINIIIWFLLLSVCLGSLIYSTAAL |
| | | | GVLMSNLGMPSYCTGYREGYLNSTNVTIAT |
| | | | YCTGSIPCSVCLSGLDSLDTYPSLETIQIT |
| | | | ISSFKWDLTAFGLVAEWFLAYILFTRFFYV |
| | | | LGLAAIMQLFFSYFAVHFISNSWLMWLIIN |
| | | | LVQMAPISAMVRMYIFFASFYYVWKSYVHV |
| | | | VDGCNSSTCMMCYKRNRATRVECTTIVNGV |
| | | | RRSFYVYANGGKGFCKLHNWNCVNCDTFCA |
| | | | GSTFISDEVARDLSLQFKRPINPTDQSSYI |
| | | | VDSVTVKNGSIHLYFDKAGQKTYERHSLSH |
| | | | FVNLDNLRANNTKGSLPINVIVFDGKSKCE |
| | | | ESSAKSASVYYSQLMCQPILLLDQALVSDV |
| | | | GDSAEVAVKMFDAYVNTFSSTFNVPMEKLK |
| | | | TLVATAEAELAKNVSLDNVLSTFISAARQG |
| | | | FVDSDVETKDVVECLKLSHQSDIEVTGDSC |
| | | | NNYMLTYNKVENMTPRDLGACIDCSARHIN |
| | | | AQVAKSHNIALIWNVKDFMSLSEQLRKQIR |
| | | | SAAKKNNLPFKLTCATTRQVVNVVTTKIAL |
| | | | KGGKIVNNWLKQLIKVTLVFLFVAAIFYLI |
| | | | TPVHVMSKHTDFSSEIIGYKAIDGGVTRDI |
| | | | ASTDTCFANKHADFDTWFSQRGGSYTNDKA |
| | | | CPLIAAVITREVGFVVPGLPGTILRTTNGD |
| | | | FLHFLPRVFSAVGNICYTPSKLIEYTDFAT |
| | | | SACVLAAECTIFKDASGKPVPYCYDTNVLE |
| | | | GSVAYESLRPDTRYVLMDGSIIQFPNTYLE |
| | | | GSVRVVTTFDSEYCRHGTCERSEAGVCVST |
| | | | SGRWVLNNDYYRSLPGVFCGVDAVNLLTNM |
| | | | FTPLIQPIGALDISASIVAGGIVAIVVTCL |
| | | | AYYFMRFRRAFGEYSHVVAFNTLLFLMSFT |
| | | | VLCLTPVYSFLPGVYSVIYLYLTFYLTNDV |
| | | | SFLAHIQWMVMFTPLVPFWITIAYIICIST |
| | | | KHFYWFFSNYLKRRVVFNGVSFSTFEEAAL |
| | | | CTFLLNKEMYLKLRSDVLLPLTQYNRYLAL |
| | | | YNKYKYFSGAMDTTSYREAACCHLAKALND |
| | | | FSNSGSDVLYQPPQTSITSAVLQSGFRKMA |
| | | | FPSGKVEGCMVQVTCGTTTLNGLWLDDVVY |
| | | | CPRHVICTSEDMLNPNYEDLLIRKSNHNFL |
| | | | VQAGNVQLRVIGHSMQNCVLKLKVDTANPK |
| | | | TPKYKFVRIQPGQTFSVLACYNGSPSGVYQ |
| | | | CAMRPNFTIKGSFLNGSCGSVGFNIDYDCV |
| | | | SFCYMHHMELPTGVHAGTDLEGNFYGPFVD |
| | | | RQTAQAAGTDTTITVNVLAWLYAAVINGDR |
| | | | WFLNRFTTTLNDFNLVAMKYNYEPLTQDHV |
| | | | DILGPLSAQTGIAVLDMCASLKELLQNGMN |
| | | | GRTILGSALLEDEFTPFDVVRQCSGVTFQS |
| | | | AVKRTIKGTHHWLLLTILTSLLVLVQSTQW |
| | | | SLFFFLYENAFLPFAMGIIAMSAFAMMFVK |
| | | | HKHAFLCLFLLPSLATVAYFNMVYMPASWV |
| | | | MRIMTWLDMVDTSLSGFKLKDCVMYASAVV |
| | | | LLILMTARTVYDDGARRVWTLMNVLTLVYK |
| | | | VYYGNALDQAISMWALIISVTSNYSGVVTT |
| | | | VMFLARGIVFMCVEYCPIFFITGNTLQCIM |
| | | | LVYCFLGYFCTCYFGLFCLLNRYFRLTLGV |
| | | | YDYLVSTQEFRYMNSQGLLPPKNSIDAFKL |
| | | | NIKLLGVGGKPCIKVATVQSKMSDVKCTSV |
| | | | VLLSVLQQLRVESSSKLWAQCVQLHNDILL |
| | | | AKDTTEAFEKMVSLLSVLLSMQGAVDINKL |
| | | | CEEMLDNRATLQAIASEFSSLPSYAAFATA |
| | | | QEAYEQAVANGDSEVVLKKLKKSLNVAKSE |
| | | | FDRDAAMQRKLEKMADQAMTQMYKQARSED |
| | | | KRAKVTSAMQTMLFTMLRKLDNDALNNIIN |
| | | | NARDGCVPLNIIPLTTAAKLMVVIPDYNTY |
| | | | KNTCDGTTFTYASALWEIQQVVDADSKIVQ |
| | | | LSEISMDNSPNLAWPLIVTALRANSAVKLQ |
| | | | NNELSPVALRQMSCAAGTTQTACTDDNALA |
| | | | YYNTTKGGRFVLALLSDLQDLKWARFPKSD |
| | | | GTGTIYTELEPPCRFVTDTPKGPKVKYLYF |
| | | | IKGLNNLNRGMVLGSLAATVRLQAGNATEV |
| | | | PANSTVLSFCAFAVDAAKAYKDYLASGGQP |
| | | | ITNCVKMLCTHTGTGQAITVTPEANMDQES |
| | | | FGGASCCLYCRCHIDHPNPKGFCDLKGKYV |
| | | | QIPTTCANDPVGFTLKNTVCTVCGMWKGYG |
| | | | CSCDQLREPMLQSADAQSFLNRVCGVSAAR |
| | | | LTPCGTGTSTDVVYRAFDIYNDKVAGFAKF |
| | | | LKTNCCRFQEKDEDDNLIDSYFVVKRHTFS |
| | | | NYQHEETIYNLLKDCPAVAKHDFFKFRIDG |
| | | | DMVPHISRQRLTKYTMADLVYALRHFDEGN |
| | | | CDTLKEILVTYNCCDDDYFNKKDWYDFVEN |
| | | | PDILRVYANLGERVRQALLKTVQFCDAMRN |
| | | | AGIVGVLTLDNQDLNGNWYDFGDFIQTTPG |
| | | | SGVPVVDSYYSLLMPILTLTRALTAESHVD |
| | | | TDLTKPYIKWDLLKYDFTEERLKLFDRYFK |
| | | | YWDQTYHPNCVNCLDDRCILHCANFNVLFS |
| | | | TVFPPTSFGPLVRKIFVDGVPFVVSTGYHF |
| | | | RELGVVHNQDVNLHSSRLSFKELLVYAADP |
| | | | AMHAASGNLLLDKRTTCFSVAALTNNVAFQ |
| | | | TVKPGNFNKDFYDFAVSKGFFKEGSSVELK |
| | | | HFFFAQDGNAAISDYDYYRYNLPTMCDIRQ |
| | | | LLFVVEVVDKYFDCYDGGCINANQVIVNNL |
| | | | DKSAGFPFNKWGKARLYYDSMSYEDQDALF |
| | | | AYTKRNVIPTITQMNLKYAISAKNRARTVA |
| | | | GVSICSTMTNRQFHQKLLKSIAATRGATVV |
| | | | IGTSKFYGGWHNMLKTVYSDVENPHLMGWD |
| | | | YPKCDRAMPNMLRIMASLVLARKHTTCCSL |
| | | | SHRFYRLANECAQVLSEMVMCGGSLYVKPG |
| | | | GTSSGDATTAYANSVFNICQAVTANVNALL |
| | | | STDGNKIADKYVRNLQHRLYECLYRNRDVD |
| | | | TDFVNEFYAYLRKHFSMMILSDDAVVCFNS |
| | | | TYASQGLVASIKNFKSVLYYQNNVFMSEAK |
| | | | CWTETDLTKGPHEFCSQHTMLVKQGDDYVY |
| | | | LPYPDPSRILGAGCFVDDIVKTDGTLMIER |
| | | | FVSLAIDAYPLTKHPNQEYADVFHLYLQYI |
| | | | RKLHDELTGHMLDMYSVMLTNDNTSRYWEP |
| | | | EFYEAMYTPHTVLQAVGACVLCNSQTSLRC |
| | | | GACIRRPFLCCKCCYDHVISTSHKLVLSVN |
| | | | PYVCNAPGCDVTDVTQLYLGGMSYYCKSHK |
| | | | PPISFPLCANGQVFGLYKNTCVGSDNVTDF |
| | | | NAIATCDWTNAGDYILANTCTERLKLFAAE |
| | | | TLKATEETFKLSYGIATVREVLSDRELHLS |
| | | | WEVGKPRPPLNRNYVFTGYRVTKNSKVQIG |
| | | | EYTFEKGDYGDAVVYRGTTTYKLNVGDYFV |
| | | | LTSHTVMPLSAPTLVPQEHYVRITGLYPTL |
| | | | NISDEFSSNVANYQKVGMQKYSTLQGPPGT |
| | | | GKSHFAIGLALYYPSARIVYTACSHAAVDA |
| | | | LCEKALKYLPIDKCSRIIPARARVECFDKF |
| | | | KVNSTLEQYVFCTVNALPETTADIVVFDEI |
| | | | SMATNYDLSVVNARLRAKHYVYIGDPAQLP |
| | | | APRTLLTKGTLEPEYFNSVCRLMKTIGPDM |
| | | | FLGTCRRCPAEIVDTVSALVYDNKLKAHKD |
| | | | KSAQCFKMFYKGVITHDVSSAINRPQIGVV |
| | | | REFLTRNPAWRKAVFISPYNSQNAVASKIL |
| | | | GLPTQTVDSSQGSEYDYVIFTQTTETAHSC |
| | | | NVNRFNVAITRAKVGILCIMSDRDLYDKLQ |
| | | | FTSLEIPRRNVATLQAENVTGLFKDCSKVI |
| | | | TGLHPTQAPTHLSVDTKFKTEGLCVDIPGI |
| | | | PKDMTYRRLISMMGFKMNYQVNGYPNMFIT |
| | | | REEAIRHVRAWIGFDVEGCHATREAVGTNL |
| | | | PLQLGFSTGVNLVAVPTGYVDTPNNTDFSR |
| | | | VSAKPPPGDQFKHLIPLMYKGLPWNVVRIK |
| | | | IVQMLSDTLKNLSDRVVFVLWAHGFELTSM |
| | | | KYFVKIGPERTCCLCDRRATCFSTASDTYA |
| | | | CWHHSIGFDYVYNPFMIDVQQWGFTGNLQS |
| | | | NHDLYCQVHGNAHVASCDAIMTRCLAVHEC |
| | | | FVKRVDWTIEYPIIGDELKINAACRKVQHM |
| | | | VVKAALLADKFPVLHDIGNPKAIKCVPQAD |
| | | | VEWKFYDAQPCSDKAYKIEELFYSYATHSD |
| | | | KFTDGVCLFWNCNVDRYPANSIVCRFDTRV |
| | | | LSNLNLPGCDGGSLYVNKHAFHTPAFDKSA |
| | | | FVNLKQLPFFYYSDSPCESHGKQVVSDIDY |
| | | | VPLKSATCITRCNLGGAVCRHHANEYRLYL |
| | | | DAYNMMISAGFSLWVYKQFDTYNLWNTFTR |
| | | | LQSLENVAFNVVNKGHFDGQQGEVPVSIIN |
| | | | NTVYTKVDGVDVELFENKTTLPVNVAFELW |
| | | | AKRNIKPVPEVKILNNLGVDIAANTVIWDY |
| | | | KRDAPAHISTIGVCSMTDIAKKPTETICAP |
| | | | LTVFFDGRVDGQVDLFRNARNGVLITEGSV |
| | | | KGLQPSVGPKQASLNGVTLIGEAVKTQFNY |
| | | | YKKVDGVVQQLPETYFTQSRNLQEFKPRSQ |
| | | | MEIDFLELAMDEFIERYKLEGYAFEHIVYG |
| | | | DFSHSQLGGLHLLIGLAKRFKESPFELEDF |
| | | | IPMDSTVKNYFITDAQTGSSKCVCSVIDLL |
| | | | LDDFVEIIKSQDLSVVSKVVKVTIDYTEIS |
| | | | FMLWCKDGHVETFYPKLQSSQAWQPGVAMP |
| | | | NLYKMQRMLLEKCDLQNYGDSATLPKGIMM |
| | | | NVAKYTQLCQYLNTLTLAVPYNMRVIHFGA |
| | | | GSDKGVAPGTAVLRQWLPTGTLLVDSDLND |
| | | | FVSDADSTLIGDCATVHTANKWDLIISDMY |
| | | | DPKTKNVTKENDSKEGFFTYICGFIQQKLA |
| | | | LGGSVAIKITEHSWNADLYKLMGHFAWWTA |
| | | | FVTNVNASSSEAFLIGCNYLGKPREQIDGY |
| | | | VMHANYIFWRNTNPIQLSSYSLFDMSKFPL |
| | | | KLRGTAVMSLKEGQINDMILSLLSKGRLII |
| | | | RENNRVVISSDVLVNN |
| 33 | YP_009724390 | surface | MFVFLVLLPLVSSQCVNLTTRTQLPPAYTN |
| | | glycoprotein | SFTRGVYYPDKVFRSSVLHSTQDLFLPFFS |
| | | [Severe acute | NVTWFHAIHVSGTNGTKRFDNPVLPFNDGV |
| | | respiratory | YFASTEKSNIIRGWIFGTTLDSKTQSLLIV |
| | | syndrome | NNATNVVIKVCEFQFCNDPFLGVYYHKNNK |
| | | coronavirus 2] | SWMESEFRVYSSANNCTFEYVSQPFLMDLE |
| | | | GKQGNFKNLREFVFKNIDGYFKIYSKHTPI |
| | | | NLVRDLPQGFSALEPLVDLPIGINITRFQT |
| | | | LLALHRSYLTPGDSSSGWTAGAAAYYVGYL |
| | | | QPRTFLLKYNENGTITDAVDCALDPLSETK |
| | | | CTLKSFTVEKGIYQTSNFRVQPTESIVRFP |
| | | | NITNLCPFGEVFNATRFASVYAWNRKRISN |
| | | | CVADYSVLYNSASFSTFKCYGVSPTKLNDL |
| | | | CFTNVYADSFVIRGDEVRQIAPGQTGKIAD |
| | | | YNYKLPDDFTGCVIAWNSNNLDSKVGGNYN |
| | | | YLYRLFRKSNLKPFERDISTEIYQAGSTPC |
| | | | NGVEGFNCYFPLQSYGFQPTNGVGYQPYRV |
| | | | VVLSFELLHAPATVCGPKKSTNLVKNKCVN |
| | | | FNFNGLTGTGVLTESNKKFLPFQQFGRDIA |
| | | | DTTDAVRDPQTLEILDITPCSFGGVSVITP |
| | | | GTNTSNQVAVLYQDVNCTEVPVAIHADQLT |
| | | | PTWRVYSTGSNVFQTRAGCLIGAEHVNNSY |
| | | | ECDIPIGAGICASYQTQTNSPRRARSVASQ |
| | | | SIIAYTMSLGAENSVAYSNNSIAIPTNFTI |
| | | | SVTTEILPVSMTKTSVDCTMYICGDSTECS |
| | | | NLLLQYGSFCTQLNRALTGIAVEQDKNTQE |
| | | | VFAQVKQIYKTPPIKDFGGFNFSQILPDPS |
| | | | KPSKRSFIEDLLFNKVTLADAGFIKQYGDC |
| | | | LGDIAARDLICAQKFNGLTVLPPLLTDEMI |
| | | | AQYTSALLAGTITSGWTFGAGAALQIPFAM |
| | | | QMAYRFNGIGVTQNVLYENQKLIANQFNSA |
| | | | IGKIQDSLSSTASALGKLQDVVNQNAQALN |
| | | | TLVKQLSSNFGAISSVLNDILSRLDKVEAE |
| | | | VQIDRLITGRLQSLQTYVTQQLIRAAEIRA |
| | | | SANLAATKMSECVLGQSKRVDFCGKGYHLM |
| | | | SFPQSAPHGVVFLHVTYVPAQEKNFTTAPA |
| | | | ICHDGKAHFPREGVFVSNGTHWFVTQRNFY |
| | | | EPQIITTDNTFVSGNCDVVIGIVNNTVYDP |
| | | | LQPELDSFKEELDKYFKNHTSPDVDLGDIS |
| | | | GINASVVNIQKEIDRLNEVAKNLNESLIDL |
| | | | QELGKYEQYIKWPWYIWLGFIAGLIAIVMV |
| | | | TIMLCCMTSCCSCLKGCCSCGSCCKFDEDD |
| | | | SEPVLKGVKLHYT |
| 34 | YP_009724391 | ORF3a protein | MDLFMRIFTIGTVTLKQGEIKDATPSDFVR |
| | | [Severe acute | ATATIPIQASLPFGWLIVGVALLAVFQSAS |
| | | respiratory | KIITLKKRWQLALSKGVHFVCNLLLLFVTV |
| | | syndrome | YSHLLLVAAGLEAPFLYLYALVYFLQSINF |
| | | coronavirus 2] | VRIIMRLWLCWKCRSKNPLLYDANYFLCWH |
| | | | TNCYDYCIPYNSVTSSIVITSGDGTTSPIS |
| | | | EHDYQIGGYTEKWESGVKDCVVLHSYFTSD |
| | | | YYQLYSTQLSTDTGVEHVTFFIYNKIVDEP |
| | | | EEHVQIHTIDGSSGVVNPVMEPIYDEPTTT |
| | | | TSVPL |
| 35 | YP_009724392 | envelope protein | MYSFVSEETGTLIVNSVLLFLAFVVFLLVT |
| | | [Severe acute | LAILTALRLCAYCCNIVNVSLVKPSFYVYS |
| | | respiratory | RVKNLNSSRVPDLLV |
| | | syndrome | |
| | | coronavirus 2] | |
| 36 | YP_009724393 | membrane | MADSNGTITVEELKKLLEQWNLVIGFLFLT |
| | | glycoprotein | WICLLQFAYANRNRFLYIIKLIFLWLLWPV |
| | | [Severe acute | TLACFVLAAVYRINWITGGIAIAMACLVGL |
| | | respiratory | MWLSYFIASFRLFARTRSMWSFNPETNILL |
| | | syndrome | NVPLHGTILTRPLLESELVIGAVILRGHLR |
| | | coronavirus 2] | IAGHHLGRCDIKDLPKEITVATSRTLSYYK |
| | | | LGASQRVAGDSGFAAYSRYRIGNYKLNTDH |
| | | | SSSSDNIALLVQ |
| 37 | YP_009724394 | ORF6 protein | MFHLVDFQVTIAEILLIIMRTFKVSIWNLD |
| | | [Severe acute | YIINLIIKNLSKSLTENKYSQLDEEQPMEI |
| | | Respiratory | D |
| | | syndrome | |
| | | coronavirus 2] | |
| 38 | YP_009724395 | ORF7a protein | MKIILFLALITLATCELYHYQECVRGTTVL |
| | | [Severe acute | LKEPCSSGTYEGNSPFHPLADNKFALTCFS |
| | | respiratory | TQFAFACPDGVKHVYQLRARSVSPKLFIRQ |
| | | syndrome | EEVQELYSPIFLIVAAIVFITLCFTLKRKT |
| | | coronavirus 2] | E |
| 39 | YP_009724396 | ORF8 protein | MKFLVFLGIITTVAAFHQECSLQSCTQHQP |
| | | [Severe acute | YVVDDPCPIHFYSKWYIRVGARKSAPLIEL |
| | | respiratory | CVDEAGSKSPIQYIDIGNYTVSCLPFTINC |
| | | syndrome | QEPKLGSLVVRCSFYEDFLEYHDVRVVLDF |
| | | coronavirus 2] | I |
| 1 | YP_009724397 | nucleocapsid | MSDNGPQNQRNAPRITFGGPSDSTGSNQNG |
| | | phosphoprotein | ERSGARSKQRRPQGLPNNTASWFTALTQHG |
| | | [Severe acute | KEDLKFPRGQGVPINTNSSPDDQIGYYRRA |
| | | respiratory | TRRIRGGDGKMKDLSPRWYFYYLGTGPEAG |
| | | syndrome | LPYGANKDGIIWVATEGALNTPKDHIGTRN |
| | | coronavirus 2] | PANNAAIVLQLPQGTTLPKGFYAEGSRGGS |
| | | | QASSRSSSRSRNSSRNSTPGSSRGTSPARM |
| | | | AGNGGDAALALLLLDRLNQLESKMSGKGQQ |
| | | | QQGQTVTKKSAAEASKKPRQKRTATKAYNV |
| | | | TQAFGRRGPEQTQGNFGDQELIRQGTDYKH |
| | | | WPQIAQFAPSASAFFGMSRIGMEVTPSGTW |
| | | | LTYTGAIKLDDKDPNFKDQVILLNKHIDAY |
| | | | KTFPPTEPKKDKKKKADETQALPQRQKKQQ |
| | | | TVTLLPAADLDDFSKQLQQSMSSADSTQA |

In some embodiments, an antigen provided herein comprises at least one polypeptide derived from a SARS-CoV-2 protein. In some embodiments, the antigen comprises an amino acid sequence that is at least about 50%, 55%, 60%, 65%, 70%, 75%, 80%, 85%, 90%, 95%, or 99% identical to SEQ ID NO: 1. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 2. In some embodiments, the antigen comprises an amino acid sequence that is at least about 50%, 55%, 60%, 65%, 70%, 75%, 80%, 85%, 90%, 95%, or 99% identical to SEQ ID NO: 3. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 4. In some embodiments, the antigen comprises an amino acid sequence that is at least about 50%, 55%, 60%, 65%, 70%, 75%, 80%, 85%, 90%, 95%, or 99% identical to SEQ ID NO: 5. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 6. In some embodiments, the antigen comprises an amino acid sequence that is at least about 50%, 55%, 60%, 65%, 70%, 75%, 80%, 85%, 90%, 95%, or 99% identical to SEQ ID NO: 7. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 8. In some embodiments, the antigen comprises an amino acid sequence that is at least about 50%, 55%, 60%, 65%, 70%, 75%, 80%, 85%, 90%, 95%, or 99% identical to SEQ ID NO: 9. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 10. In some embodiments, the antigen comprises an amino acid sequence that is at least about 50%, 55%, 60%, 65%, 70%, 75%, 80%, 85%, 90%, 95%, or 99% identical to SEQ ID NO: 11. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 12. In some embodiments, the antigen comprises an amino acid sequence that is at least about 50%, 55%, 60%, 65%, 70%, 75%, 80%, 85%, 90%, 95%, or 99% identical to SEQ ID NO: 13. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 14. In some embodiments, the antigen comprises an amino acid sequence that is at least about 50%, 55%, 60%, 65%, 70%, 75%, 80%, 85%, 90%, 95%, or 99% identical to SEQ ID NO: 15. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 16. In some embodiments, the antigen comprises an amino acid sequence that is at least about 50%, 55%, 60%, 65%, 70%, 75%, 80%, 85%, 90%, 95%, or 99% identical to SEQ ID NO: 17. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 18. In some embodiments, the antigen comprises an amino acid sequence that is at least about 50%, 55%, 60%, 65%, 70%, 75%, 80%, 85%, 90%, 95%, or 99% identical to SEQ ID NO: 19. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 20. In some embodiments, the antigen comprises an amino acid sequence that is at least about 50%, 55%, 60%, 65%, 70%, 75%, 80%, 85%, 90%, 95%, or 99% identical to SEQ ID NO: 21. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 22. In some embodiments, the antigen comprises an amino acid sequence that is at least about 50%, 55%, 60%, 65%, 70%, 75%, 80%, 85%, 90%, 95%, or 99% identical to SEQ ID NO: 23. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 24. In some embodiments, the antigen comprises an amino acid sequence that is at least about 50%, 55%, 60%, 65%, 70%, 75%, 80%, 85%, 90%, 95%, or 99% identical to SEQ ID NO: 25. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 26. In some embodiments, the antigen comprises an amino acid sequence that is at least about 50%, 55%, 60%, 65%, 70%, 75%, 80%, 85%, 90%, 95%, or 99% identical to SEQ ID NO: 27. In some embodiments, the at least one polypeptide comprises an amino acid sequence that is at least about 50%, 55%, 60%, 65%, 70%, 75%. 80%, 85%, 90%, 95% or 99% identical to SEQ ID NO: 28. In some embodiments, the antigen comprises an amino acid sequence that is at least about 50%, 55%, 60%, 65%, 70%, 75%, 80%, 85%, 90%, 95%, or 99% identical to SEQ ID NO: 29. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 30. In some embodiments, the antigen comprises an amino acid sequence that is at least about 50%, 55%, 60%, 65%, 70%, 75%, 80%, 85%, 90%, 95%, or 99% identical to SEQ ID NO: 31. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 32. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 33. In some embodiments, the antigen comprises an amino acid sequence that is at least about 50%, 55%, 60%, 65%, 70%, 75%, 80%, 85%, 90%, 95%, or 99% identical to SEQ ID NO: 34. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 35. In some embodiments, the antigen comprises an amino acid sequence that is at least about 50%, 55%, 60%, 65%, 70%, 75%, 80%, 85%, 90%, 95%, or 99% identical to SEQ ID NO: 36. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 37. In some embodiments, the antigen comprises an amino acid sequence that is at least about 50%, 55%, 60%, 65%, 70%, 75%, 80%, 85%, 90%, 95%, or 99% identical to SEQ ID NO: 38. In some embodiments, the at least one polypeptide comprises an amino acid sequence that is at least about 50%, 55%, 60%, 65%, 70%, 75%, 80%, 85%, 90%, 95%, or 99% identical to SEQ ID NO: 39.

In some embodiments, the antigen comprises at least one polypeptide comprising 5, 6, 7, 8, 9, 10, 11, 12 13, or 15 consecutive amino acids of SEQ ID NO: 1. In some embodiments, the antigen comprises at least one polypeptide comprising 5, 6, 7, 8, 9, 10, 11, 12 13, or 15 consecutive amino acids of SEQ ID NO: 2. In some embodiments, the antigen comprises at least one polypeptide comprising 5, 6, 7, 8, 9, 10, 11, 12 13, or 15 consecutive amino acids of SEQ ID NO: 3. In some embodiments, the antigen comprises at least one polypeptide comprising 5, 6, 7, 8, 9, 10, 11, 12 13, or 15 consecutive amino acids of SEQ ID NO: 4. In some embodiments, the antigen comprises at least one polypeptide comprising 5, 6, 7, 8, 9, 10, 11, 12 13, or 15 consecutive amino acids of SEQ ID NO: 5. In some embodiments, the antigen comprises at least one polypeptide comprising 5, 6, 7, 8, 9, 10, 11, 12 13, or 15 consecutive amino acids of SEQ ID NO: 6. In some embodiments, the antigen comprises at least one polypeptide comprising 5, 6, 7, 8, 9, 10, 11, 12 13, or 15 consecutive amino acids of SEQ ID NO: 7. In some embodiments, the antigen comprises at least one polypeptide comprising 5, 6, 7, 8, 9, 10, 11, 12 13, or 15 consecutive amino acids of SEQ ID NO: 8. In some embodiments, the antigen comprises at least one polypeptide comprising 5, 6, 7, 8, 9, 10, 11, 12 13, or 15 consecutive amino acids of SEQ ID NO: 9. In some embodiments, the antigen comprises at least one polypeptide comprising 5, 6, 7, 8, 9, 10, 11, 12 13, or 15 consecutive amino acids of SEQ ID NO: 10. In some embodiments, the antigen comprises at least one polypeptide comprising 5, 6, 7, 8, 9, 10, 11, 12 13, or 15 consecutive amino acids of SEQ ID NO: 11. In some embodiments, the antigen comprises at least one polypeptide comprising 5, 6, 7, 8, 9, 10, 11, 12 13, or 15 consecutive amino acids of SEQ ID NO: 12. In some embodiments, the antigen comprises at least one polypeptide comprising 5, 6, 7, 8, 9, 10, 11, 12 13, or 15 consecutive amino acids of SEQ ID NO: 13. In some embodiments, t the antigen comprises at least one polypeptide comprising 5, 6, 7, 8, 9, 10, 11, 12 13, or 15 consecutive amino acids of SEQ ID NO: 14. In some embodiments, the antigen comprises at least one polypeptide comprising 5, 6, 7, 8, 9, 10, 11, 12 13, or 15 consecutive amino acids of SEQ ID NO: 15. In some embodiments, the antigen comprises at least one polypeptide comprising 5, 6, 7, 8, 9, 10, 11, 12 13, or 15 consecutive amino acids of SEQ ID NO: 16. In some embodiments, the antigen comprises at least one polypeptide comprising 5, 6, 7, 8, 9, 10, 11, 12 13, or 15 consecutive amino acids of SEQ ID NO: 17. In some embodiments, the antigen comprises at least one polypeptide comprising 5, 6, 7, 8, 9, 10, 11, 12 13, or 15 consecutive amino acids of SEQ ID NO: 18. In some embodiments, the antigen comprises at least one polypeptide comprising 5, 6, 7, 8, 9, 10, 11, 12 13, or 15 consecutive amino acids of SEQ ID NO: 19. In some embodiments, the antigen comprises at least one polypeptide comprising 5, 6, 7, 8, 9, 10, 11, 12 13, or 15 consecutive amino acids of SEQ ID NO: 20. In some embodiments, the antigen comprises at least one polypeptide comprising 5, 6, 7, 8, 9, 10, 11, 12 13, or 15 consecutive amino acids of SEQ ID NO: 21. In some embodiments, the antigen comprises at least one polypeptide comprising 5, 6, 7, 8, 9, 10, 11, 12 13, or 15 consecutive amino acids of SEQ ID NO: 22. In some embodiments, the antigen comprises at least one polypeptide comprising 5, 6, 7, 8, 9, 10, 11, 12 13, or 15 consecutive amino acids of SEQ ID NO: 23. In some embodiments, the antigen comprises at least one polypeptide comprising 5, 6, 7, 8, 9, 10, 11, 12 13, or 15 consecutive amino acids of SEQ ID NO: 24. In some embodiments, the antigen comprises at least one polypeptide comprising 5, 6, 7, 8, 9, 10, 11, 12 13, or 15 consecutive amino acids of SEQ ID NO: 25. In some embodiments, the antigen comprises at least one polypeptide comprising 5, 6, 7, 8, 9, 10, 11, 12 13, or 15 consecutive amino acids of SEQ ID NO: 26. In some embodiments, the antigen comprises at least one polypeptide comprising 5, 6, 7, 8, 9, 10, 11, 12 13, or 15 consecutive amino acids of SEQ ID NO: 27. In some embodiments, the antigen comprises at least one polypeptide comprising 5, 6, 7, 8, 9, 10, 11, 12 13, or 15 consecutive amino acids of SEQ ID NO: 28. In some embodiments, the antigen comprises at least one polypeptide comprising 5, 6, 7, 8, 9, 10, 11, 12 13, or 15 consecutive amino acids of SEQ ID NO: 29. In some embodiments, the antigen comprises at least one polypeptide comprising 5, 6, 7, 8, 9, 10, 11, 12 13, or 15 consecutive amino acids of SEQ ID NO: 30. In some embodiments, the antigen comprises at least one polypeptide comprising 5, 6, 7, 8, 9, 10, 11, 12 13, or 15 consecutive amino acids of SEQ ID NO: 31. In some embodiments, the antigen comprises at least one polypeptide comprising 5, 6, 7, 8, 9, 10, 11, 12 13, or 15 consecutive amino acids of SEQ ID NO: 32. In some embodiments, the antigen comprises at least one polypeptide comprising 5, 6, 7, 8, 9, 10, 11, 12 13, or 15 consecutive amino acids of SEQ ID NO: 33. In some embodiments, the antigen comprises at least one polypeptide comprising 5, 6, 7, 8, 9, 10, 11, 12 13, or 15 consecutive amino acids of SEQ ID NO: 34. In some embodiments, the antigen comprises at least one polypeptide comprising 5, 6, 7, 8, 9, 10, 11, 12 13, or 15 consecutive amino acids of SEQ ID NO: 35. In some embodiments, the antigen comprises at least one polypeptide comprising 5, 6, 7, 8, 9, 10, 11, 12 13, or 15 consecutive amino acids of SEQ ID NO: 36. In some embodiments, the antigen comprises at least one polypeptide comprising 5, 6, 7, 8, 9, 10, 11, 12 13, or 15 consecutive amino acids of SEQ ID NO: 37. In some embodiments, the antigen comprises at least one polypeptide comprising 5, 6, 7, 8, 9, 10, 11, 12 13, or 15 consecutive amino acids of SEQ ID NO: 38. In some embodiments, the antigen comprises at least one polypeptide comprising 5, 6, 7, 8, 9, 10, 11, 12 13, or 15 consecutive amino acids of SEQ ID NO: 39.

In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 1. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 2. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 3. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 4. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 5. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 6. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 7. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 8. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 9. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 10. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 11. In some embodiments, t the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 12. In some embodiments the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 13. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 14. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 15. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 16. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 17. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 18. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 19. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 20. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 21. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 22. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 23. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 24. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 25. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 26. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 27. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 28. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 29. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 30. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 31. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 32. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 33. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 34. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 35. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 36. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 37. In some embodiments the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 38. In some embodiments, the antigen comprises at least one polypeptide comprising 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive amino acids of SEQ ID NO: 39.

In some embodiments, the antigen comprises at least one polypeptide comprising an amino acid sequence that is at least about 50%, 55%, 60%, 65%, 70%, 75%, 80%, 85%, 90%, 95%, or 99% identical to a sequence as set forth in Table 1.

```
Exemplary SARS-CoV-2 nucleocapsid protein
(SEQ ID NO: 1)
    1 msdngpqnqr napritfggp sdstgsnqng ersgarskqr rpqglpnnta swftaltqhg
  61 kedlkfprgq gvpintnssp ddqigyyrra trrirggdgk mkdlsprwyf yylgtgpeag
121 lpygankdgi iwvategaln tpkdhigtrn pannaaivlq lpqgttlpkg fyaegsrggs
181 qassrsssrs rnssrnstpg ssrgtsparm agnggdaala lllldrlnql eskmsgkgqq
241 qqgqtvtkks aacaskkprq krtatkaynv tqafgrrgpe qtqgnfgdqe lirqgtdykh
301 wpqiaqfaps asaffgmsri gmevtpsgtw ltytgaikld dkdpnfkdqv illnkhiday
361 ktfpptepkk dkkkkadetq alpqrqkkqq tvtllpaadl ddfskqlqqs mssadstqa
Exemplary mosaic SARS-CoV-2 antigen
(SEQ ID NO: 2)
ADPEFRLSWY DPDFQARLTR SNSKCQGQLE VYLKDGWHMV CSQSWGRSSK QWEDPSQASK
VCQRLNCGVP LSLGPFLVTY TPQSSIICYG QLGSFSNCSH SRNDMCHSLG LTCLEPQKTT
PPTTRPPPTT TPEPTAPPRL QLVAQSGGQH CAGVVEFYSG SLGGTISYEA QDKTQDLENF
LCNNLQCGSF LKHLPETEAG RAQDPGEPRE HQPLPIQWKI QNSSCTSLEH CFRKIKPQKS
GRVLALLCSG FQPKVQSRLV GGSSICEGTV EVRQGAQWAA LCDSSSARSS LRWEEVCREQ
QCGSVNSYRV LDAGDPTSRG LFCPHQKLSQ CHELWERNSY CKKVFVTCQD PNPHHHHHH.
```

### Pathogens

Antigens and compositions provided herein can be used to detect immune response to specific pathogens by a subject. As used herein, a pathogen can include viruses, bacteria, fungi, helminths, protozoa, or parasites, e.g. malarial parasites, Plasmodium parasites, Toxoplasma parasites, and Schistosoma parasites, roundworms, heartworms, phytophagous nematodes, flukes, Acanthocephala, and tapeworms. In some embodiments, a pathogen is a mammalian pathogen. In some embodiments, a pathogen is a human pathogen. In some embodiments, a pathogen is a non-human animal pathogen, for example, a non-human primate pathogen.

In some embodiments, an antigen provided herein can be used to detect immune response to a viral pathogen. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

In some embodiments, an antigen provided herein can be used to detect immune response to a bacterial pathogen. Exemplary bacterial pathogen includes Escherichia coli group, including Enterotoxigenic Escherichia coli (ETEC), Enteropathogenic Escherichia coli (EPEC), Enterohemorrhagic Escherichia coli (EHEC), Enteroinvasive Escherichia coli (EIEC), Salmonella spp., Campylobacter jejuni, Listeria, Yersinia enterocolitica, Shigella spp., Vibrio parahaemolyticus, Coxiella burnetii, Mycobacterium bovis, Brucella spp., Vibrio cholera, Vibrio vulnificus, Cronobacter, Aeromonas hydrophila and other spp., Plesiomonas shigelloides, Clostridium perfringens, Clostridium botulinum, Staphylococcus aureus, Bacillus cereus and other Bacillus spp., Listeria monocytogenes, Streptococcus spp., Enterococcus, and others.

In some embodiments, an antigen provided herein comprises a peptide derived from a viral peptide. Examples of the virus which the at least one polypeptide is derived from includes: cytomegalo virus, Herpex Simplex, Epstein-Barr virus, Simian virus 40, Bovine papillomavirus, Adeno-associated virus, Adenovirus, Vaccinia virus, and Baculo virus, Coronavirus, Semliki Forest virus, Sindbis virus, Poko virus, Rabies virus, Influenza virus, SV5, Respiratory Syncytial virus. Venezuela equine encephalitis virus, Kunjin virus, Sendai virus, Vesicular stomatitisvirus, Retroviruses, Papovaviridae, Adenoviridae, Herpesviridae, Herpesvirales, Ascoviridae, Ampullaviridae, Asfarviridae, Baculoviridae, Fuselloviridae, Globuloviridae, Guttaviridae, Hytrosaviridae, Iridoviridae, Lipothrixviridae, Nimaviridae, Poxviridae, Tectiviridae, Corticoviridae, Sulfolobus, Caudovirales, Corticoviridae, Tectiviridaea, Ligamenvirales, Ampullaviridae, Bicaudaviridae, Clavaviridae, Fuselloviridae, Globuloviridae, Guttaviridae, Turriviridae, Ascovirus, Baculovirus, Hytrosaviridae, Iridoviridae, Polydnaviruses, Mimiviridae, Marseillevirus, Megavirus, Mavirus virophage, Sputnik virophage, Nimaviridae, Phycodnaviridae, pleolipoviruses, Plasmaviridae, Pandoraviridae, Dinodnavirus, Rhizidiovirus, Salterprovirus, Sphaerolipoviridae, Anelloviridae, Bidnaviridae, Circoviridae, Geminiviridae, Genomoviridae, Inoviridae, Microviridae, Nanoviridae, Parvoviridae, Spiraviridae, Amalgaviridae, Birnaviridae, Chrysoviridae, Cystoviridae, Endornaviridae, Hypoviridae, Megabimaviridae, Partitiviridae, Picobirnaviridae, Quadriviridae, Reoviridae, Totiviridae, Nidovirales, Picomavirales, Tymovirales, Mononegavirales, Bornaviridae, Filoviridae, Mymonaviridae, Nyamiviridae, Paramyxoviridae, Pneumoviridae, Rhabdoviridae, Sunviridae, Anphevirus, Arlivirus, Chengtivirus, Crustavirus, Wastrivirus, Bunyavirales, Feraviridae, Fimoviridae, Hantaviridae, Jonviridae, Nairoviridae, Peribunyaviridae, Phasmaviridae, Phenuiviridae, Tospoviridae, Arenaviridae, Ophioviridae, Orthomyxoviridae, Deltavirus, Taastrup virus, Alpharetrovirus, Avian leukosis virus; Rous sarcoma virus, Betaretrovirus, Mouse mammary tumor virus, Gammaretrovirus, Murine leukemia virus, Feline leukemia virus, Bovine leukemia virus, Human T-lymphotropic virus, Epsilonretrovirus, Walleye dermal sarcoma virus, Lentivirus, Human immunodeficiency virus 1, Simian and Feline immunodeficiency viruses, Spumavirus, Simian foamy virus, Orthoretrovirinae, Spumaretrovirinae, Metaviridae, Pseudoviridae, Retroviridae, Hepadnaviridae, or Caulimoviridae.

In some embodiments, the viral peptide is derived from a coronavirus. In some embodiments, the coronavirus is selected from the group consisting of: alphacoronavirus, betacoronavirus, deltacoronavirus, and gammacoronavirus. Additional examples of coronavirus includes MERS-CoV, SARS-CoV, and SARS-CoV-2. In some embodiments, the at least one polypeptide is derived from a viral protein of SARS-CoV-2.

### Immune Response

In some embodiments, the composition comprising the at least one antigen formulated to screen for an immune response by a subject. In some embodiments, the immune response is induced by activity of macrophage. In some embodiments, the immune response is induced by activity of monocyte. In some embodiments, the immune response is induced by activity of T cell. In some embodiments, the immune response is induced by activity of B cell. In some embodiments, the immune response involves proliferation of an immune cell, for example, a T cell. In some embodiments, the immune response involves activation of an immune cell, for example, a T cell. In some embodiments, the immune response involves proliferation or activation of CD4+ cytotoxic T cells. In some embodiments, the immune response involves proliferation or activation of CD8+ cytotoxic T cells. In some embodiments, an antigen provided herein is processed by an immune cell, for example, an antigen presenting cell.

In some embodiments, a composition provided herein comprises a sample obtained from a subject. For example, a sample obtained from a body fluid of a subject may be incubated with at least one antigen provided herein for detection of an immune response by immune cells in the body fluid. In some embodiments, a sample obtained from a subject comprises immune cells capable of producing immune response molecules or related expression or metabolic products following stimulation with an appropriate antigen. For example, a sample may include T cells capable of producing immune response molecules, such as cytokines, following stimulation with an antigen of a pathogen, e.g. a viral antigen. The sample may be obtained from a subject that was previously exposed to the viral antigen, developed symptoms associated with exposure to the virus, diagnosed with an infection of the virus, developed no symptoms associated with exposure to the virus, or may be obtained from a healthy individual.

Examples of immune cells include but are not limited to lymphocytes, NK cells, T-cells, B-cells, macrophages and monocytes, dendritic cells, any other immune cell capable of producing one or more immune effector molecules in response to direct or indirect antigen stimulation. In some embodiments, a sample obtained from a subject comprises lymphocytes. In some embodiments, a sample obtained from a subject comprises T-cells. In some embodiments, a sample obtained from a subject comprises CD4+ helper T-cells. In some embodiments, a sample obtained from a subject comprises CD8+ cytotoxic T-cells. In some embodiments, a sample obtained from a subject comprises T-cells capable of eliciting an immune response upon recognition of an antigen as described herein. Without wishing to be bound by any theory, T-cells previously exposed to an antigen in a detection composition described herein, or an antigen for which the antigen in a detection composition is representative may be able to initiate to re-stimulation of the T-cells with specific memory of the antigen. For example, a subject previously exposed to a non-SARS-CoV-2 virus, e.g., a “common cold” virus, or a different coronavirus, may have developed T cell immunity against SARS-CoV-2 due to cross-reactive T cell recognition. In some embodiments, stimulated antigen-specific T-cells produce immune-response molecules, for example, immunoproteins. The immune-response molecules may be used as indicators or analytes to detect an immune response as described herein.

In some embodiments, a comprises stimulator cells, e.g. antigen presenting cells which are capable of presenting the tested antigen to the T-cells. Without wishing to be bound by any theory, antigen presenting cells (APCs) may take up intracellular and extracellular antigens for proteolytic processing to generate epitopes that are then presented on major histocompatibility complexes (MHC) on the surface of APCs. Epitopes that are displayed by MHC on antigen presenting cells may be cleavage peptides or products of larger peptide or protein antigen precursors, e.g., antigens taken up from the detection composition. In some embodiments, MHC classes III epitopes displayed at the surface of APCs are able to stimulate an immune response upon recognition by a CD4+ or CD8+ T cell. In some embodiments, APCs may be artificial antigen presenting cells or particles. For example, stimulator cells such as irradiated autologous or HLA matched antigen-presenting cells can be separately added to the detection composition during incubation with a sample which then present the antigen to T-cells. Artificial antigen presenting embodiments include but are not limited to particles or lipid vesicles with associated recombinant MHC molecules or peptides and recombinant co-stimulatory molecules.

### Test Compositions and Containers

In some embodiments, the composition described herein comprises an antigen and a buffer, a solvent, or a formulation. In some embodiments, an antigen is in a liquid solution. In some embodiments, an antigen is lyophilized.

Compositions for detection immune responses described herein may comprise antigens and additional components. Each composition may be maintained in an individual container or containers for detection purposes. In some embodiments, an antigen is in a liquid composition comprising at least one polypeptide in a concentration that is between about 0.1 nanogram (ng) to 100 ng of the at least one polypeptide in a 1 milliliter (ml) volume of the composition. In some embodiments, the concentration is in an amount sufficient to induce an immune response in a sample obtained from a subject. In some embodiments, the concentration is in an amount sufficient to induce immune cell proliferation in a sample obtained from a subject. In some embodiments, the concentration of the amount of the at least one polypeptide per 1 milliliter volume is at least or equal to about 0.1 ng/ml to about 100 ng/ml. In some embodiments, the concentration of the amount of the at least one polypeptide per 1 milliliter volume is at least or equal to about 0.1 ng/ml to about 0.2 ng/ml, about 0.1 ng/ml to about 0.5 ng/ml, about 0.1 ng/ml to about 1 ng/ml, about 0.1 ng/ml to about 2 ng/ml, about 0.1 ng/ml to about 5 ng/ml, about 0.1 ng/ml to about 10 ng/ml, about 0.1 ng/ml to about 20 ng/ml, about 0.1 ng/ml to about 50 ng/ml, about 0.1 ng/ml to about 100 ng/ml, about 0.2 ng/ml to about 0.5 ng/ml, about 0.2 ng/ml to about 1 ng/ml, about 0.2 ng/ml to about 2 ng/ml, about 0.2 ng/ml to about 5 ng/ml, about 0.2 ng/ml to about 10 ng/ml, about 0.2 ng/ml to about 20 ng/ml, about 0.2 ng/ml to about 50 ng/ml, about 0.2 ng/ml to about 100 ng/ml, about 0.5 ng/ml to about 1 ng/ml, about 0.5 ng/ml to about 2 ng/ml, about 0.5 ng/ml to about 5 ng/ml, about 0.5 ng/ml to about 10 ng/ml, about 0.5 ng/ml to about 20 ng/ml, about 0.5 ng/ml to about 50 ng/ml, about 0.5 ng/ml to about 100 ng/ml, about 1 ng/ml to about 2 ng/ml, about 1 ng/ml to about 5 ng/ml, about 1 ng/ml to about 10 ng/ml, about 1 ng/ml to about 20 ng/ml, about 1 ng/ml to about 50 ng/ml, about 1 ng/ml to about 100 ng/ml, about 2 ng/ml to about 5 ng/ml, about 2 ng/ml to about 10 ng/ml, about 2 ng/ml to about 20 ng/ml, about 2 ng/ml to about 50 ng/ml, about 2 ng/ml to about 100 ng/ml, about 5 ng/ml to about 10 ng/ml, about 5 ng/ml to about 20 ng/ml, about 5 ng/ml to about 50 ng/ml, about 5 ng/ml to about 100 ng/ml, about 10 ng/ml to about 20 ng/ml, about 10 ng/ml to about 50 ng/ml, about 10 ng/ml to about 100 ng/ml, about 20 ng/ml to about 50 ng/ml, about 20 ng/ml to about 100 ng/ml, or about 50 ng/ml to about 100 ng/ml. In some embodiments, the concentration of the amount of the at least one polypeptide per 1 milliliter volume is at least or equal to about 0.1 ng/ml, about 0.2 ng/ml, about 0.5 ng/ml, about 1 ng/ml, about 2 ng/ml, about 5 ng/ml, about 10 ng/ml, about 20 ng/ml, about 50 ng/ml, or about 100 ng/ml. In some embodiments, the concentration of the amount of the at least one polypeptide per 1 milliliter volume is at least at least about 0.1 ng/ml, about 0.2 ng/ml, about 0.5 ng/ml, about 1 ng/ml, about 2 ng/ml, about 5 ng/ml, about 10 ng/ml, about 20 ng/ml, or about 50 ng/ml.

In some embodiments, the composition comprising the at least one polypeptide is formulated to be a solid or powder composition. In some embodiments, the solid or powder composition is lyophilized. In some instances, the solid or powder composition comprises about 0.1 ug to 100 ug of the at least one polypeptide in a container of the composition. In some embodiments, a container comprises the at least one polypeptide in a lyophilized form in an amount at least or equal to about 0.1 ug to about 100 ug. In some embodiments, a container comprises the at least one polypeptide in a lyophilized form in an amount at least or equal to about 0.1 ug to about 0.2 ug, about 0.1 ug to about 0.5 ug, about 0.1 ug to about 1 ug, about 0.1 ug to about 2 ug, about 0.1 ug to about 5 ug, about 0.1 ug to about 10 ug, about 0.1 ug to about 20 ug, about 0.1 ug to about 50 ug, about 0.1 ug to about 100 ug, about 0.2 ug to about 0.5 ug, about 0.2 ug to about 1 ug, about 0.2 ug to about 2 ug, about 0.2 ug to about 5 ug, about 0.2 ug to about 10 ug, about 0.2 ug to about 20 ug, about 0.2 ug to about 50 ug, about 0.2 ug to about 100 ug, about 0.5 ug to about 1 ug, about 0.5 ug to about 2 ug, about 0.5 ug to about 5 ug, about 0.5 ug to about 10 ug, about 0.5 ug to about 20 ug, about 0.5 ug to about 50 ug, about 0.5 ug to about 100 ug, about 1 ug to about 2 ug, about 1 ug to about 5 ug, about 1 ug to about 10 ug, about 1 ug to about 20 ug, about 1 ug to about 50 ug, about 1 ug to about 100 ug, about 2 ug to about 5 ug, about 2 ug to about 10 ug, about 2 ug to about 20 ug, about 2 ug to about 50 ug, about 2 ug to about 100 ug, about 5 ug to about 10 ug, about 5 ug to about 20 ug, about 5 ug to about 50 ug, about 5 ug to about 100 ug, about 10 ug to about 20 ug, about 10 ug to about 50 ug, about 10 ug to about 100 ug, about 20 ug to about 50 ug, about 20 ug to about 100 ug, or about 50 ug to about 100 ug. In some embodiments, a container comprises the at least one polypeptide in a lyophilized form in an amount at least or equal to about 0.1 ug, about 0.2 ug, about 0.5 ug, about 1 ug, about 2 ug, about 5 ug, about 10 ug, about 20 ug, about 50 ug, or about 100 ug. In some embodiments, each dose of the solid or powder composition comprises the at least one polypeptide in an amount at least at least or equal to about 0.1 ug, about 0.2 ug, about 0.5 ug, about 1 ug, about 2 ug, about 5 ug, about 10 ug, about 20 ug, or about 50 ug.

Antigens and polypeptides provided herein can have long term stability or shelf-lives. In some embodiments, an antigen in a container as described herein is stable for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 60, 90, 120, 150, 180, 210, 240, 270, 300, 330, 360, or 365 or more days after formulated or lyophilized.

A container comprising the composition for detecting immune responses as described herein may comprise an anticoagulation agent. Without wishing to be bound by any theory, anticoagulation agents allow for incubation of samples obtained from subjects, e.g. blood samples, with one or more antigens provided herein. In some embodiments, the anticoagulation agent is heparin, citrate, unfractionated heparin, bivalirudin, or hirudin. In some embodiments, the anticoagulation agent is lithium heparin. In some embodiments, the contained comprises approximately 1, 2, 5, 10, 15, 25, 30, 35, 40, 45, 50, 60, 70, 80, 90, 100, 150, 200 USP units of heparin.

In some embodiments, the composition is formulated to comprise one or more amino acids. In some embodiments, the composition is formulated to comprise approximately about 0.01 μg, about 0.02 μg, about 0.05 μg, about 0.1 μg, about 0.2 μg, about 0.3 μg, about 0.5 μg, about 0.75 μg, about 1 μg, about 2 μg, about 3 μg, about 4 μg, about 5 μg, about 10 μg, about 25 μg, about 50 μg, about 75 μg, about 100 μg, about 200 μg, about 250 μg, about 300 μg, about 400 μg, about 500 μg, about 750 μg, about 1000 μg of amino acids. In some embodiments, the composition is formulated to comprise glutamine, arginine, asparagine, cystine, leucine, isoleucine, lysine hydrochloride, serine, aspartic acid, glutamic acid, hydroxyproline, proline, threonine, tyrosine, valine, histidine, methionine, phenylalanine, glycine; tryptophan, glutathione, or any combination thereof. In some embodiments, the composition is formulated to comprise one or more vitamins. In some embodiments, the composition is formulated to comprise approximately about 0.01 μg, about 0.02 μg, about 0.05 μg, about 0.1 μg, about 0.2 μg, about 0.3 μg, about 0.5 μg, about 0.75 μg, about 1 μg, about 2 μg, about 3 μg, about 4 μg, about 5 μg, about 10 μg, about 25 μg, about 50 μg, about 75 μg, about 100 μg, about 200 μg, about 250 μg, about 300 μg, about 400 μg, about 500 μg, about 750 μg, about 1000 μg of vitamins. In some embodiments, the composition is formulated to comprise inositol; choline chloride; para-aminobenzoic acid, folic acid, nicotinamide, pyridoxine hydrochloride, thiamine hydrochloride; calcium pantothenate; biotin, riboflavin, yanocobalamin, or any combination thereof. In some embodiments, the composition is formulated to comprise one or more salt. In some embodiments, the composition is formulated to comprise sodium chloride, sodium bicarbonate, disodium phosphate, potassium chloride, magnesium sulfate, aluminum phosphate, calcium nitrate, or any combination thereof. In some embodiments, the composition is formulated to comprise approximately about 0.1 mg, about 0.2 mg, about 0.3 mg, about 0.4 mg, about 0.5 mg, about 0.75 mg, about 1 mg, about 2 mg, about 3 mg, about 4 mg, about 5 mg, about 10 mg, about 20 mg, about 30 mg, about 40 mg, about 50 mg, about 60 mg, about 70 mg, about 80 mg, about 90 mg, or about 100 mg of sodium chloride, sodium bicarbonate, disodium phosphate, potassium chloride, magnesium sulfate, and calcium nitrate, or any combination thereof.

In some embodiments, the composition is formulated to include one or more salt premixed with an antigen described herein. In some embodiments, the composition is formulated to include an adjuvant premixed with the antigen. In some embodiments, the adjuvant and the antigen are premixed at a ratio of 0.25:1, 0.5:1, 0.6:1, 0.75:1, 0.8:1, 0.9:1, 1:1, 1.1:1, 1:25:1, 1.3:1, 1.4:1, 1.5:1, 1.75:1, 2:1, 2.25:1, 2.5:1, 2.75:1, 3:1, 3.5:1, 4:1, 5:1, 5.5:1, 6:1 or higher by weight. Weight ratio of an adjuvant may be measured by weight of the salt ion, for example, Aluminum3+ in the adjuvant. In some embodiments, the adjuvant and the antigen are premixed at a ratio of 0.25:1, 0.5:1, 0.6:1, 0.75:1, 0.8:1, 0.9:1, 1:1, 1.1:1, 1:25:1, 1.3:1, 1.4:1, 1.5:1, 1.75:1, 2:1, 2.25:1, 2.5:1, 2.75:1, 3:1, 3.5:1, 4:1, 5:1, 5.5:1, 6:1, 7.5:1, 10:1, 20:1, 25:1, 30:1, 35:1, 40:1, 50:1, 60:1, 70:1, 80:1, 90:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, 1000:1 or higher by molar ratio.

In some embodiments, the adjuvant and the antigen are premixed at a ratio of 1:1 by weight. In some embodiments, the adjuvant salt component and the antigen are premixed at a ratio between 1:1 to 3:1 by weight. In some embodiments, the adjuvant is an aluminum adjuvant or an aluminum salt. In some embodiments, the adjuvant comprises aluminum hydroxide, for example, crystalline aluminum hydroxide. In some embodiments, the adjuvant comprises an aluminum hydroxide adjuvant. In some embodiments, the adjuvant comprises an aluminum phosphate adjuvant. In some embodiments, the adjuvant comprises an alumen, e.g. aluminum potassium sulfate or AlK(SO₄)₂. In some embodiments, the adjuvant comprises Adju-Phos®. In some embodiments, the adjuvant comprises Alhydrogel®. Additional examples of adjuvants include, but are not limited to, Calcium phosphate, MPLA (monophosphoryl lipid A), MF59, AS03 (alpha-tocopherol, squalene, and polysorbate 80), AS04 (mixture of monophosphoryl lipid A (MPL) and aluminum hydroxide), Incomplete Freund's adjuvant, Cochleates, Virus-like particles, Virosomes, PLA (polylactic acid), PLG (poly[lactide-coglycolide]), dsRNA: Poly(I:C), Poly-IC:LC, LPS, Flagellin, Imidazoquinolines, imiquimod (R837), resiquimod (848), gardiquimod, CpG oligodeoxynucleotides (ODN), Muramyl dipeptide (MDP), Saponins (QS-21), Chitosan, or any combination thereof.

Without wishing to be bound by any theory, adjuvants premixed with antigens provided herein may be incubated with a sample obtained from a subject for fast detection of immune responses. In some embodiments, an antigen premixed with an adjuvant is incubated with a sample for equal to or less than 30 minutes before quantification of immune responses of cells in the sample. In some embodiments, an antigen premixed with an adjuvant described herein is incubated with a sample for equal to or less than 5, 10, 15, 20, 25, 30, 35, 40, 45, 50, 55, 60 minutes before quantification of immune responses of cells in the sample. In some embodiments, an antigen premixed with an adjuvant described herein is incubated with a sample for equal to or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours before quantification of immune responses of cells in the sample. In some embodiments, an antigen premixed with an adjuvant described herein is incubated with a sample for equal to or less than 1, 2, 3, 4, 5, 6, 7, 8, 9, 10, 11, or 12 days before quantification of immune responses of cells in the sample. Without wishing to be bound by any theory, antigens premixed with an adjuvant as described herein may allow for shorter incubation time for detection and quantification of an immune response as compared to same antigens not premixed with an adjuvant. In some embodiments, detection and quantification of the immune response is determined by a statistically significant change in an analyte as described herein compared to a control sample or control incubation.

In some embodiments, the composition is formulated to comprise one or more sugar. In some embodiments, the composition is formulated to comprise approximately about 0.1 mg, about 0.2 mg, about 0.3 mg, about 0.4 mg, about 0.5 mg, about 0.75 mg, about 1 mg, about 2 mg, about 3 mg, about 4 mg, about 5 mg, about 10 mg, about 20 mg, about 30 mg, about 40 mg, about 50 mg, about 60 mg, about 70 mg, about 80 mg, about 90 mg, or about 100 mg of sugar. In some embodiments, the composition is formulated to comprise a reducing sugar, e.g. glucose, galactose, glyceraldehyde, fructose, ribose, xylose, or any combination thereof. In some embodiments, the composition does not contain a non-reducing sugar. In some embodiments, the composition is formulated to comprise approximately about 0.1 mg, about 0.2 mg, about 0.3 mg, about 0.4 mg, about 0.5 mg, about 0.75 mg, about 1 mg, about 2 mg, about 3 mg, about 4 mg, about 5 mg, about 10 mg, about 20 mg, about 30 mg, about 40 mg, about 50 mg, about 60 mg, about 70 mg, about 80 mg, about 90 mg, or about 100 mg of a sugar. In some embodiments, the sugar is a reducing sugar. In some embodiments, the container comprising the composition for immune response detection or the composition does not include a chelator. In some embodiments, the container comprising the composition for immune response detection or the composition does not include EDTA.

The composition for detection of an immune response may or may not contain a serum component. In some embodiments, the container comprising the composition for immune response detection includes a serum supplement, e.g. a bovine serum albumin. In some embodiments, detection compositions comprising a serum supplement are used for detection with certain sample types, e.g., saliva. In some embodiments, the composition is formulated to comprise approximately about 0.01 μg, about 0.02 μg, about 0.05 μg, about 0.1 μg, about 0.2 μg, about 0.3 μg, about 0.5 μg, about 0.75 μg, about 1 μg, about 2 μg, about 3 μg, about 4 μg, about 5 μg, about 10 μg, about 25 μg, about 50 μg, about 75 μg, about 100 μg, about 200 μg, about 250 μg, about 300 μg, about 400 μg, about 500 μg, about 750 μg, about 1000 μg, about 0.1 mg, about 0.2 mg, about 0.3 mg, about 0.4 mg, about 0.5 mg, about 0.75 mg, about 1 mg, about 2 mg, about 3 mg, about 4 mg, about 5 mg, about 10 mg, about 20 mg, about 30 mg, about 40 mg, about 50 mg, about 60 mg, about 70 mg, about 80 mg, about 90 mg, or about 100 mg of a serum supplement. In some embodiments, the container comprising the composition for immune response detection or the composition is formulated to not include a serum supplement.

In some instances, the composition includes pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as sodium bicarbonate and ammonium chloride. Such acids, bases and buffers are included in an amount required to maintain pH of the composition in an acceptable range.

In some instances, the composition includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

A composition described herein can comprise samples obtained from a subject. For example, a whole blood sample obtained from a subject may be incubated with the composition and antigens described herein to detect immune response by the subject to a specific pathogen. A sample maybe obtained from different parts of a subject. In some embodiments, a sample is obtained from a biofluid of a subject and subsequently diluted with a buffer. In some embodiments, a sample is obtained from a biofluid of a subject and not diluted. In some embodiments, a sample comprises PBMCs, basophils, eosinophils, RBCs, WBCs, neutrophils, monocytes, macrophages, lymphocytes, platelets, CD8 T cells, NK cells, CD4 T cells, B cells, or any combination thereof. In some embodiments, a sample comprises a body fluid. In some embodiments, the body fluid can be blood, saliva, tears, serum, cerebral fluid, cerebrospinal fluid, urine, sweat, amniotic fluid, synovial fluid, pleural fluid, pericardial fluid, peritoneal fluid, lymph fluid, bone marrow, thymus fluid, nasal fluid, pulmonary fluid, or bronchoalveolar lavage. In some embodiments, a sample comprises saliva obtained from a subject. In some embodiments, a sample comprises buffy coat obtained from a subject. In some embodiments, a sample comprises whole blood obtained from a subject. In some embodiments, a sample comprises plasma obtained from a subject.

Compositions for detection of immune response provided herein can be in various volumes and containers. In some embodiments, a container comprises about luL, 2 uL, 5 uL, 7.5 uL, 10 uL, 15 uL, 20 uL, 25 uL, 30 uL, 35 uL, 40 uL, 45 uL, or 50 uL of a sample, e.g. whole blood, obtained from a subject. In some embodiments, a container comprises about 1 mL, 2 mL, 5 mL, 7.5 mL, 10 mL, 15 mL, 20 mL, 25 L, 30 mL, 35 mL, 40 mL, 45 mL, 50 mL or more of a sample, e.g. whole blood, obtained from a subject. A container may comprise various materials and of various volumes. In some embodiments, a container is a steril glass tube. In some embodiments, a container is a steril polycarbonate tube. In some embodiments, a container is a microfuge tube, a cryotube, a cryovial, a matrix tube, or an ampoule. In some embodiments, a container is a storage plate. In some embodiments, a container comprises a multi-well plate, e.g. a 48 well plate or a 96 well plate for high throughput detection and analysis. In some embodiments, the container is a vessel.

A sample may be obtained by techniques known to those skilled in the art, for example, venipuncture, IVS, or capillary tube sampling. In some embodiments, a capillary tube is used to collect blood from the surface of the skin by capillary action. In some embodiments, the sample is collected from a subject into a collection vessel containing at least one antigen and at least one anticoagulant, e.g. lithium heparin, or to which an antigen, and an anticoagulant, e.g. lithium heparin, is subsequently added. In some embodiments, blood is sampled using a capillary sampling device such as a pin prick device and blood is collected into a heparinised collecting container and subsequently transferred into an appropriate container for co-incubation with the antigen and the anticoagulation agent. In some embodiments the antigen and the anticoagulant are provided in form of a single composition as described above. In some embodiments, whole blood from a subject is collected into a container containing the antigen and the anti-coagulant. In some embodiments, the antigen and the anticoagulant are added to the whole blood sample after collection.

### Sample Analysis

Disclosed herein, in some embodiments, are methods for screening and detecting an immune response in a subject to a pathogen or a composition described herein (e.g., antigenic peptide).

Without wishing to be bound by any theory, immune cells taken from a subject, when exposed to an antigen derived from a pathogen, e.g. a viral antigen, may be activated within a time period (e.g., 24-72 hours) which indicates a presence of the adaptive immune response.

In some embodiments, a sample obtained from a subject is incubated with an antigen or composition provided herein to detect an immune response to the antigen. In some embodiments, a sample is incubated with the antigen for at least 1 hour. In some embodiments, a sample is incubated with the antigen for at least about 2, 3, 4, 5, 6, 7, 8, 9, 10, 12, 14, 16, 18, 20, 22, 24, 26, 28, 30, 35, 40, 45, 50, 60, 65, 66, 67, 68, 69, 70, 71, 72 hours or more. In some embodiments, a sample is incubated with the antigen for about 2 hours. In some embodiments, a sample is incubated with the antigen for about 4 hours. In some embodiments, a sample is incubated with the antigen for about 6 hours. In some embodiments, a sample is incubated with the antigen for about 12 hours. In some embodiments, a sample is incubated with the antigen for about 24 hours. In some embodiments, a sample is incubated with the antigen for about 48 hours. In some embodiments, a sample is incubated with the antigen for about 2 to 50 hours, 2 to 40 hours, 5 to 30 hours, 8 to 24 hours, 16 to 24 hours, or a time period in between including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 hours. In some embodiments, an antigen premixed with an adjuvant is incubated with a sample for equal to or less than 30 minutes before quantification of immune responses of cells in the sample. In some embodiments, a sample is incubated with the antigen for equal to or less than 5, 10, 15, 20, 25, 30, 35, 40, 45, 50, 55, 60 minutes before quantification of immune responses of cells in the sample. In some embodiments, a sample is incubated with the antigen for equal to or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours before quantification of immune responses of cells in the sample. In some embodiments, a sample is incubated with the antigen for at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours before quantification of immune responses of cells in the sample. In some embodiments, a sample is incubated with the antigen for equal to or less than 1, 2, 3, 4, 5, 6, 7, 8, 9, 10, 11, or 12 days before quantification of immune responses of cells in the sample.

In some embodiments, the time determine of whether a sample is positive or negative by the analyte response is determined between 1-96 hours. In some embodiments, the time is at least 1 hour. In some embodiments, the time is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours. In some embodiments, the time is no more than 48, 72, or 96 hours.

Detection and quantification of the immune response is determined by a statistically significant change in an analyte as described herein compared to a control sample or control incubation. A significant change can be a threshold value, or cut-off value, of analyte expression of the sample compared to a control sample, wherein the cut-off value is the fold-change of analyte expression of the sample over the control sample. In some embodiments, the cut-off value is between 1.5 and 5.0 fold change in expression of the analyte. In some embodiments, the cut-off value is at least about 1.5 fold change in expression. In some embodiments, the cut-off value can be about 5.0 fold change. In some embodiments, the cut-off value is about at least 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 fold change in expression of the analyte. In some embodiments, the cut-off value is at least a 2.0 fold change in expression of the analyte.

In some embodiments, a sample is incubated with the antigen at a temperature of about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C. or 45° C. In some embodiments, a sample is incubated with the antigen at a temperature of above 30° C. In some embodiments, a sample is incubated with the antigen at a temperature of about 35° C. In some embodiments, a sample is incubated with the antigen at a temperature of about 30° C.

In some embodiments, a sample is incubated with at least one antigen described herein. The at least one antigen may comprise one or more peptides derived from one or more pathogens. In some embodiments, methods described herein comprise identifying an immune response by a subject to an epitope or peptide derived from a pathogen protein. In some embodiments, the epitope or peptide is derived from a virus. In some embodiments, the peptide comprises epitopes or amino acids derived from more than one structural or function proteins of a virus. In some embodiments, the peptide is derived from a receptor binding domain (RBD) or receptor binding motif of SARS-CoV-2 or a fragment or variant thereof. In some embodiments, the peptide is derived from a spike protein of SARS-CoV-2 or a fragment or variant thereof. In some embodiments, the peptide is derived from a nucleocapsid protein of SARS-CoV-2 or a fragment or variant thereof.

Immune response may be detected by quantifying an indicator of immune cells in a sample incubated with an antigen or peptide in a composition described herein. In some embodiments, the indicator may be the quantity, activity, or expression level of one or more analytes in the sample. In some embodiments, the or more analytes are gene products of a cell in the sample. An analyte may be a protein, a nucleic acid, a DNA, a RNA, a metabolite, a sugar, a lipid, or any combination thereof. In some embodiments, the indicator is an amount of an analyte, e.g., a protein. In some embodiments, the indicator is an amount of an analyte, e.g., a nucleic acid encoding a protein. In some embodiments, the indicator is an amount of a nucleic acid. In some embodiments, the indicator is an amount of a metabolite. In some embodiments, the indicator is an amount of an immunoprotein. In some embodiments, the indicator is an amount of one or more different immunoproteins. In some embodiments, the indicator is a ratio between two immunoproteins or among two or more immunoproteins. In some embodiments, the indicator is an amount of a nucleic acid, e.g. a mRNA encoding an immunoprotein. In some embodiments, the indicator is an amount of one or more nucleic acids, e.g. mRNAs encoding different immunoproteins. In some embodiments, the indicator is a ratio between nucleic acids, e.g. mRNAs encoding two immunoproteins or among nucleic acids, e.g. mRNAs encoding two or more immunoproteins. In some embodiments, an analyte is a protein or a mRNA produced by an immune cell. In some embodiments, an analyte is related to immune cell proliferation. In some embodiments, an analyte is related to T cell proliferation or activation. In some embodiments, an analyte is related to CD4+ T cell proliferation or activation. In some embodiments, an analyte is related to CD8+ T cell proliferation or activation. In some embodiments, the analyte is lymphokine, an interleukin, a monokine, a cytotoxin, a exotoxin, or a chemokine, or a mRNA encoding the same. In some embodiments, the analyte is a cytokine or a mRNA encoding the cytokine. In some embodiments, the analyte is a interferon or a mRNA encoding the interferon. In some embodiments, the analyte is a interferon-gamma (IFN-gamma) or a mRNA encoding the interferon-gamma. In some embodiments, the analyte is an immune response molecule. For example, the analyte may be CCR7, TNF, IL-2, CD127, CCL19, CCL21, KLRG-1, IL-21, IL21Ra, or IL-17. In some embodiments, the analyte is a proliferation indicator. For example, the analyte may be Ki-67 or PD-1. In some embodiments, the analyte is a immune effector protein. For example, the analyte may be perforin, or granzyme. Additional analytes that may be quantified include but are not limited to: IL-4, IL-6, IL-8 (CXCL8), IL-10, IL-12, IL-13, IL-16 (LCF), IL-1α (IL-1F1), IL-1β (IL-1F2), IL-1rα (IL-1F3), Tumor Necrosis Factor alpha (TNF-α), TNF-β, TNF-γ, CD252, CD154, CD178, CD70, CD153, 4-1 BBL, CD253, CD254, APO3L, CD256, CD257, CD258, TL-1, GITRL, Ectodysplasin, Transforming Growth Factor beta (TGF-0), Colony Stimulating Factor (CSF, such as Granulocyte (G)-CSF or Granulocyte Macrophage (GM)-CSF), complement component 5a (C5a), Groa (CXCL1), sICAM-1 (CD54), IP-10 (CXCL10), I-TAC (CXCL11), MCP-1 (CCL2), MIF (GIF), MIP-1α (CCL3), MIP-1β(CCL4), Serpin E1 (PAI-1), RANTES (CCL5), MIG (CXCL9).

Samples obtained from a subject contain multiple levels of immunological and biological information and analytes that may be quantified and analyzed by compositions and methods provided herein. For example, a sample obtained from a subject may comprise immune cells as well as metabolites from a body fluid of a subject, a foreign pathogen that the subject is exposed to, or any combination thereof. Accordingly, in an aspect, also provided herein are methods and compositions for detecting a pathogen in a sample obtained from a subject. In some embodiments, a pathogen is a virus, the nucleic acid or protein component of which may be detected with compositions and methods provided herein. In some embodiments, the pathogen is a coronavirus. In some embodiments, the pathogen is a SARS-CoV-2. Existence of a pathogen, e.g. a SARS-CoV-2 in a sample of a subject may be detected with any one of the quantification techniques described herein. For example, primers may be designed specifically for SARS-CoV-2 RNA for detection of the virus in a given sample. Therefore, also provided herein are methods for detecting current infection or exposure state to a pathogen of a subject.

In another aspect, high-throughput quantification analysis may be utilized to analyze an immunological status, inflammation status or any other physiological status of a subject using a sample obtained from the subject with quantification methods provided herein. In some embodiments, a sample obtained from a subject may be incubated with an antigen as provided herein. The total RNA or protein of the incubation container may be isolated and analyzed for genome, transcriptome, and proteome wide analysis. Expression level of genes encoding immune response related analytes may be determined with RNA-seq or nanopore sequencing. The total RNA or protein of the incubation container may also be isolated and analyzed for existence of any pathogen, for example, a virus.

Analytes for detecting immune response can be measured with known techniques. For example, amount of a protein in a sample incubated in a container can be quantified with enzyme-linked immunosorbent assay (ELISA), mass spectrometry, blotting, western blotting, immunohistochemistry, or any combination thereof.

Amount of nucleic acids, e.g. mRNAs can be quantified with RNA sequencing, DNA sequencing (e.g., sequencing of complementary deoxyribonucleic acid (cDNA) obtained from RNA), quantitative RT-PCR, next generation (Next-Gen) sequencing, nanopore sequencing, pyrosequencing, or Nanostring sequencing. Gene expression product levels may be normalized to an internal standard such as total messenger ribonucleic acid (mRNA) or the expression level of a particular gene.

Nucleic acid analytes may be analysed with Loop-mediated isothermal amplification (LAMP). LAMP assay as described in Notomi et al. (Nucleic Acids Res 28:E63, 2000) is incorporate herein by reference in its entirety. A LAMP assay may be used for amplification of a nucleic acid utilizing 4-6 primers designed to amplify the gene target through creation of stem-loop structures that aid in synthesizing new DNA by the polymerase. RNA analytes, e.g. mRNAs, may be analyzed with Reverse-transcription LAMP (RT-LAMP) as described in Li et al., Virol J 8:360, 2011; Parida et al., Clin Microbiol 43:2895-2903, 2005; Parida et al., Clin Microbiol 45:351-357, 2007; Parida et al., Clin Microbiol 44:4172-4178, 2006; Peyrefitte et al., Clin Microbiol 46:3653-3659, 2008; Teoh et al., BMC Infect Dis 13:387, 2013; Toriniwa and Komiya, Microbiol Immunol 50:379-387, 2006; Wheeler et al., PLoS One 2016; 11:e0147962, each of which is incorporated herein in its entirety. RT-LAMP reactions may result in the synthesis of large amounts of target nucleic acid, allowing for detection of target nucleic acid from relatively small sample volumes. In some embodiments, the large amount of amplification product allows for detection visually or by simple detectors. In some embodiments, a pH-sensitive indicator dye is used for detection of amplification product. Examples of ways to detect amplification include, but are not limited to (i) fluorescence, using DNA intercalating dyes, fluorescent molecular beacon probes or a fluorescence metal indicator such as calcein; (ii) colorimetry, using a colored indicator for alkaline metal ions, such as hydroxy naphthol blue (Goto et al., BioTechniques 46(3): 167-172, 2009) or pH indicators (Tanner et al., BioTechniques 58(2):59-68, 2015); (iii) turbidity, as the RT-LAMP reaction produces large amounts of magnesium pyrophosphate (a white precipitate) and dsDNA, which allow visual inspection of results using a turbidimeter (Mon et al., Biochem Biophys Res Commun, 289(1):150-154, 2001); (iv) electrochemically, using a pH meter for direct measurement of released hydrogen ions during the RT-LAMP procedure (Xie et al., Chem Comnum 50(100): 15932-15935, 2014), or using integrated electrodes for measuring decreases in current resulting from increasing binding of electrochemically-active DNA-binding redox reporters, such as Methylene Blue, to RT-LAMP reaction products (Xie et al., Biosens Bioelectron 55:324-329, 2014); (v) enzyme-linked immunosorbent assays (ELISA) or lateral flow immunoassays based on the use of specific probes (Tsai et al., J Virol Methods 181(1): 117-124, 2012; Ravan and Yazdanparast, Anal Biochem 439(2): 102-108, 2013); (vi) bioluminescence, through bioluminescent output of the coupled conversion of inorganic pyrophosphate produced stoichiometrically during nucleic acid synthesis to ATP by the enzyme ATP sulfurylase (Gandelman et al., PLoS ONE 5(11):e14155, 2010).

Nucleic acid analytes may be sequenced using nanopore sequencing (e.g. as described in Soni et. al. Clin Chem 53: 1996-2001, (2007), or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in each one of U.S. Pat. Nos. 5,795,782, 6,015,714, 6,627,067, 7,238,485 and 7,258,838 and U.S. patent application publications US2006003171 and US20090029477 are herein incorporated by reference in its entirety.

Nucleic acid analytes may be sequenced using Nanostring sequencing, e.g., as described in Geiss et. al. Nature Biotechnology 2007, 26(3): 317-325 or as described by NanoString Technologies). Nanostring sequencing and the like may comprise an amplification-free assay that measures nucleic acid content by counting molecules directly. Nucleic acid samples may be processed on a Nanostring instrument comprising a sequencing card and a flow cell surface. Specific capture probe pairs may be hybridized to fragmented DNA or RNA molecules from nucleic acid sample material. These captured nucleic acid molecules, with a sequencing window of up to 100 bp, may undergo sample processing, during which the core captured targets may be purified and pooled. Purified and pooled targets may then be transferred to a sequencing card where they are hybridized to the flow cell surface. Sequencing may be accomplished through multiple sequencing cycles which involve cyclic nucleic acid hybridization of targets with sequencing probes, followed by readout with reporter probes. Sequencing probes may contain a hexamer sequencing domain and a reporter domain, where sequencing domain forms the complement to the target to be sequenced, and the reporter domain may be a cyclically-read barcode. The reporter domain encoding the identity of the hexamer sequence hybridized to the target may be read via hybridization with fluorescently labeled reporter probes. Hexamer sequences derived from each single target molecule may be assembled using a graph-based algorithm and the resulting contiguous sequence reads are output into an industry-standard data output file (BAM or CRAM) that includes sequence quality metrics. Nanostring sequencing technology is disclosed in U.S. Pat. Nos. 9,381,563, 7,941,279, 8,415,102, 9,376,712, 9,856,519, 10,077,466, and U.S. patent application publication No. US20180346972, each of which is incorporated herein by reference in its entirety.

Nucleic analytes may be sequence using Nanopore technologies (Oxford Nanopore). Nanopore sequencing is one method of rapidly determining the sequence of nucleic acid molecules. Nanopore sequencing is based on the property of physically sensing the individual nucleotides (or physical changes in the environment of the nucleotides (i.e., electric current)) within an individual polynucleotide (e.g., DNA and RNA) as it traverses through a nanopore aperture. In principle, the sequence of a polynucleotide can be determined from a single molecule. However, in practice, it is preferred that a polynucleotide sequence be determined from a statistical average of data obtained from multiple passages of the same molecule or the passage of multiple molecules having the same polynucleotide sequence. The use of membrane channels to characterize polynucleotides as the molecules pass through the small ion channels has been studied by Kasianowicz et al. (Proc. Natl. Acad. Sci. USA. 93:13770-3, 1996, which is incorporated herein by reference in its entirety) by using an electric field to force single stranded RNA and DNA molecules through a 2.6 nanometer diameter nanopore aperture (i.e., ion channel) in a lipid bilayer membrane. The diameter of the nanopore aperture permitted only a single strand of a polynucleotide to traverse the nanopore aperture at any given time. As the polynucleotide traversed the nanopore aperture, the polynucleotide partially blocked the nanopore aperture, resulting in a transient decrease of ionic current. Since the length of the decrease in current is directly proportional to the length of the polynucleotide, Kasianowicz et al. were able to determine experimentally lengths of polynucleotides by measuring changes in the ionic current.

Baldarelli et al. (U.S. Pat. No. 6,015,714) and Church et al. (U.S. Pat. No. 5,795,782) describe the use of nanopores to characterize polynucleotides including DNA and RNA molecules on a monomer by monomer basis, both of which is incorporated herein by reference in its entirety. In particular, Baldarelli et al. characterized and sequenced the polynucleotides by passing a polynucleotide through the nanopore aperture. The nanopore aperture is imbedded in a structure or an interface, which separates two media. As the polynucleotide passes through the nanopore aperture, the polynucleotide alters an ionic current by blocking the nanopore aperture. As the individual nucleotides pass through the nanopore aperture, each base/nucleotide alters the ionic current in a manner that allows the identification of the nucleotide transiently blocking the nanopore aperture, thereby allowing one to characterize the nucleotide composition of the polynucleotide and perhaps determine the nucleotide sequence of the polynucleotide.

Gene expression product analytes or markers may be determined by microarray analysis using, for example, Affymetrix arrays, cDNA microarrays, oligonucleotide microarrays, spotted microarrays, or other microarray products from Biorad, Agilent, or Eppendorf. Microarrays may contain a large number of genes or alternative splice variants that may be assayed in a single experiment. In some cases, the microarray device may contain the entire human genome or transcriptome or a substantial fraction thereof allowing a comprehensive evaluation of gene expression patterns, genomic sequence, or alternative splicing. Markers may be found using standard molecular biology and microarray analysis techniques as described in Sambrook Molecular Cloning a Laboratory Manual 2001 and Baldi, P., and Hatfield, W. G., DNA Microarrays and Gene Expression 2002.

Microarray analysis may begin with extracting and purifying nucleic acid from a biological sample, (e.g. a biopsy or fine needle aspirate). For expression and alternative splicing analysis it may be advantageous to isolate and/or purify RNA from a sample. It may further be advantageous to extract and/or purify mRNA from other forms of RNA such as tRNA and rRNA

Purified nucleic acid may further be labeled with a fluorescent label, radionuclide, or chemical label such as biotin, digoxigenin, or digoxin for example by reverse transcription, polymerase chain reaction (PGR), ligation, chemical reaction or other techniques. The labeling may be direct or indirect which may further require a coupling stage. The coupling stage can occur before hybridization, for example, using ammoallyl-UTP and NHS amino-reactive dyes (like cyanine dyes) or after, for example, using biotin and labelled streptavidin. In one example, modified nucleotides (e.g. at a 1 aaUTP: 4 TTP ratio) may be added enzymatically at a lower rate compared to normal nucleotides, typically resulting in 1 every 60 bases (measured with a spectrophotometer). The aaDNA may then be purified with, for example, a column or a diafiltration device. The aminoallyl group is an amine group on a long linker attached to the nucleobase, which reacts with a reactive label (e.g. a fluorescent dye).

The labeled samples may then be mixed with a hybridization solution which may contain sodium dodecyl sulfate (SDS), SSC, dextran sulfate, a blocking agent (such as COT1 DNA, salmon sperm DNA, calf thymus DNA, PolyA or PolyT), Denhardt's solution, formamine, or a combination thereof.

A hybridization probe may be a fragment of nucleic acid, e.g., DNA or RNA of variable length, which may be used to detect in DNA or RNA samples the presence of nucleotide sequences (the DNA target) that are complementary to the sequence in the probe. The labeled probe may be first denatured (by heating or under alkaline conditions) into single DNA strands and then hybridized to the target DNA.

Loop-mediated isothermal amplification (LAMP) uses a series of primers recognizing multiple, distinct regions of a target template for a highly specific amplification reaction. Target amplification and detection of a gene can be achieved in a single step instead of using multiple denaturing, annealing, elongation steps as with traditional PCR. The LAMP assay includes primers that can form a loop structure to facilitate additional rounds of amplification by allowing annealing of additional primers. The resulting LAMP products of the gene of interest can be a very long concatemer (>20 kb) consisting of numerous repeats of the amplified target linked by the loop forming primer. The product can then be detected by multiple methods, such as a fluorescent or colorimetric assay. LAMP assay may be performed with one or more primers specific for an antigen or a pathogen of interest. For example, primers may be designed for nucleic acid sequences specific for a pathogen antigen or an epitope. In some embodiments, the LAMP assay is performed with a primer set designed for specific amplification of a sequence in a viral mRNA. In some embodiments, the LAMP assay is performed with a primer set designed for specific amplification of a sequence in a corona virus mRNA. In some embodiments, the LAMP assay is performed with a primer set designed for specific amplification of a sequence in a SARS-CoV-2 mRNA. In some embodiments, the LAMP assay is performed with a primer set as set for the in Table 2.

TABLE 2
LAMP assay primer examples

| SEQ | | |
|---|---|---|
| ID | Primer | |
| NO: | number | Sequence |
| 40 | F1 | TCAATATAGAACCGAAAAGTCGAGA |
| 41 | F2 | CAACTTCTTTGGCTTAATTCTCTC |
| 42 | F3 | GCTTGATACAAGAACTACTGAT |
| 43 | B1 | CTGTTACTGCCAGGACCCAT |
| 44 | B2 | TCCAGTAAGTCTACATCGC |
| 45 | B3 | TTACCTTGAGAAAAGAATCCG |
| 46 | 543F3- | GAACTACTGATTTCAACTTCTTTG |
| | IFNg | |
| 47 | 543B3- | GCATTATTTTTCTGTCACTCTCC |
| | IFNg | |
| 48 | 543FIP- | GAACCCAAAACGATGCAGAGCCT |
| | IFNg | TAATTCTCTCGGAAACGATGA |
| 49 | 543BIP- | CTGTTACTGCCAGGACCCATATGT |
| | IFNg | CTTTCCAATTCTTCAAAATGCC |
| 50 | LF1- | ATATAGAACCGAAAAGTCGAGACG |
| | IFNg | |
| 51 | LB1- | GCAGGTCATTCAGATGTAGCG |
| | IFNg | |
| 52 | F3-IL2 | GGATGCAACTCCTGTCTTG |
| 53 | B3-IL2 | GTTCTGTGGCCTTCTTGG |
| 54 | FIP-IL2 | CCAGTTGTAGCTGTGTTTTCTTTG |
| | | TGCACTAAGTCTTGCACTTG |
| 55 | BIP-IL2 | AGCATTTACTGCTGGATTTACAGA |
| | | TGTTGTGAGCATCCTGGT |
| 56 | F2-IL2 | TGCACTAAGTCTTGCACTTG |
| 57 | F1c-IL2 | CCAGTTGTAGCTGTGTTTTCTTTG |
| 58 | B2-IL2 | TGTTGTGAGCATCCTGGT |
| 59 | B1c-IL2 | AGCATTTACTGCTGGATTTACAGA |

Also provided herein are kits for detection of an immune response to an antigen or pathogen as described herein using a sample obtained from a subject. In some embodiments, a kit comprises a set of oligonucleotide primers specific for an analyte indicating immune response to an antigen. In some embodiments, a kit comprises a set of primers specific for an analyte indicating immune response to an antigen of a virus. In some embodiments, a kit comprises a set of primers specific for an analyte indicating immune response to an antigen of a corona virus. In some embodiments, a kit comprises a set of primers specific for an analyte indicating immune response to an antigen of SARS-Cov-2. In some embodiments, the kit comprises an oligonucleotide primer having a sequence at least 90%, at least 95%, at least 96%, at least 97%, at least 98% or at least 99% identical to TCAATATAGAACCGAAAAGTCGAGA (SEQ ID NO: 40). In some embodiments, the kit comprises an oligonucleotide primer having a sequence at least 90%, at least 95%, at least 96%, at least 97%, at least 98% or at least 99% identical to CAACTTCTTTGGCTTAATTCTCTC (SEQ ID NO: 41). In some embodiments, the kit comprises an oligonucleotide primer having a sequence at least 90%, at least 95%, at least 96%, at least 97%, at least 98% or at least 99% identical to GCTTGATACAAGAACTACTGAT (SEQ ID NO: 42). In some embodiments, the kit comprises an oligonucleotide primer having a sequence at least 90%, at least 95%, at least 96%, at least 97%, at least 98% or at least 99% identical to CTGTTACTGCCAGGACCCAT (SEQ ID NO: 43). In some embodiments, the kit comprises an oligonucleotide primer having a sequence at least 90%, at least 95%, at least 96%, at least 97%, at least 98% or at least 99% identical to TCCAGTAAGTCTACATCGC (SEQ ID NO: 44). In some embodiments, the kit comprises an oligonucleotide primer having a sequence at least 90%, at least 95%, at least 96%, at least 97%, at least 98% or at least 99% identical to TTACCTTGAGAAAAGAATCCG (SEQ ID NO: 45).

In some embodiments, a kit comprises a set of primers specific for IFN-gamma (IFNg) indicating immune response to an antigen of SARS-Cov-2. In some embodiments, the kit comprises an oligonucleotide primer having a sequence at least 90%, at least 95%, at least 96%, at least 97%, at least 98% or at least 99% identical to GAACTACTGATTTCAACTTCTTTG (SEQ ID NO: 46). In some embodiments, the kit comprises an oligonucleotide primer having a sequence at least 90%, at least 95%, at least 96%, at least 97%, at least 98% or at least 99% identical to GCATTATTTTTCTGTCACTCTCC (SEQ ID NO: 47). In some embodiments, the kit comprises an oligonucleotide primer having a sequence at least 90%, at least 95%, at least 96%, at least 97%, at least 98% or at least 99% identical to GCTTGATACAAGAACTACTGAT (SEQ ID NO: 42). In some embodiments, the kit comprises an oligonucleotide primer having a sequence at least 90%, at least 95% at least 96%, at least 97%, at least 98% or at least 99% identical to GAACCCAAAACGATGCAGAGCCTTAATTCTCTCGGAAACGATGA (SEQ ID NO: 48). In some embodiments, the kit comprises an oligonucleotide primer having a sequence at least 90%, at least 95%, at least 96%, at least 97%, at least 98% or at least 99% identical to CTGTTACTGCCAGGACCCATATGTCTTTCCAATTCTTCAAAATGCC (SEQ ID NO: 49). In some embodiments, the kit comprises an oligonucleotide primer having a sequence at least 90%, at least 95%, at least 96%, at least 97%, at least 98% or at least 99% identical to ATATAGAACCGAAAAGTCGAGACG (SEQ ID NO: 50). In some embodiments, the kit comprises an oligonucleotide primer having a sequence at least 90%, at least 95%, at least 96%, at least 97%, at least 98% or at least 99% identical to GCAGGTCATTCAGATGTAGCG (SEQ ID NO: 51).

In some embodiments, a kit comprises a set of primers specific for interleukin 2 (IL-2) indicating immune response to an antigen of SARS-Cov-2. In some embodiments, the kit comprises an oligonucleotide primer having a sequence at least 90%, at least 95%, at least 96%, at least 97%, at least 98% or at least 99% identical to GGATGCAACTCCTGTCTTG (SEQ ID NO: 52). In some embodiments, the kit comprises an oligonucleotide primer having a sequence at least 90%, at least 95%, at least 96%, at least 97%, at least 98% or at least 99% identical to GTTCTGTGGCCTTCTTGG (SEQ ID NO: 53). In some embodiments, the kit comprises an oligonucleotide primer having a sequence at least 90%, at least 95%, at least 96%, at least 97%, at least 98% or at least 99% identical to CCAGTTGTAGCTGTGTTTTCTTTGTGCACTAAGTCTTGCACTTG (SEQ ID NO: 54). In some embodiments, the kit comprises an oligonucleotide primer having a sequence at least 90%, at least 95%, at least 96%, at least 97%, at least 98% or at least 99% identical to AGCATTTACTGCTGGATTTACAGATGTTGTGAGCATCCTGGT (SEQ ID NO: 55). In some embodiments, the kit comprises an oligonucleotide primer having a sequence at least 90%, at least 95%, at least 96%, at least 97%, at least 98% or at least 99% identical to TGCACTAAGTCTTGCACTTG (SEQ ID NO: 56). In some embodiments, the kit comprises an oligonucleotide primer having a sequence at least 90%, at least 95%, at least 96%, at least 97%, at least 98% or at least 99% identical to CCAGTTGTAGCTGTGTTTTCTTTG (SEQ ID NO: 57). In some embodiments, the kit comprises an oligonucleotide primer having a sequence at least 90%, at least 95%, at least 96%, at least 97%, at least 98% or at least 99% identical to TGTTGTGAGCATCCTGGT (SEQ ID NO: 58). In some embodiments, the kit comprises an oligonucleotide primer having a sequence at least 90%, at least 95%, at least 96%, at least 97%, at least 98% or at least 99% identical to AGCATTTACTGCTGGATTTACAGA (SEQ ID NO: 59).

In some embodiments, a kit comprises one or more of the oligonucleotide primers and/or antigens provided in one or more containers or in one or more individual wells of a multi-well plate or card. Antigens and/or oligonucleotide primers may be provided suspended in an aqueous solution or as a freeze-dried or lyophilized powder, for instance. The container(s) in which the compositions comprising the antigens or nucleic acid(s) can be any conventional container that is capable of holding the supplied form, for instance, microfuge tubes, multi-well plates, ampoules, or bottles. The kits can include either labeled or unlabeled nucleic acid primers for use in amplification and/or detection an analyte, such as by RT-LAMP.

In some embodiments, the method comprises dividing the sample into at least two fractions, where one of the two fractions is incubated in a control container. In some embodiments, a sample is divided into two fractions, where a first fraction is incubated in a container with an antigen described herein and a second fraction is incubated in a container with a negative control (e.g. saline buffer). In some embodiments, a sample is divided into three fractions, where a first fraction is incubated in a container with an antigen described herein, a second fraction is incubated in a container with a negative control (e.g. saline buffer), and a third fraction incubated in a container with a positive control, such as a container including a T cell activator (e.g. mitogen). Accordingly, the method comprises analyzing an physiological state, e.g. an immune response of a subject by comparing the quantity of an analyte in the fraction of sample obtained from the subject incubated with the antigen (the test sample) with a positive and/or a negative control. In some embodiments, the antigen-dependent immune response of the test sample is determined by subtracting the analyte level determined in the negative control sample from the analyte level determined in the test sample. In some embodiments, detection and quantification of the immune response is determined by a statistically significant change in the analyte level compared to a negative control sample. In some embodiments, detection and quantification of the immune response (or, e.g. lack of specific immune response to the antigen, or immune deficiency) is determined by a statistically significant change in the analyte level compared to a positive control sample.

The method provided herein can comprise a sensitivity that would be amenable for its use a diagnostic to determine whether a subject has been previously exposed to a pathogen as previously described herein. By “sensitivity,” it is meant that the proportion of positive results that are correctly identified (i.e., subjects who are correctly identified to have been previously exposed to a pathogen as described herein). In some cases, it may be referred to as a true positive rate. In some embodiments, the method of the present disclosure comprises a sensitivity of at least 80%, 82%, 84%, 86%, 88%, 90%, 92%, 94%, 95%, 96%, 97%, 98%, 99%, or 100%. In some embodiments, the method of the present disclosure comprises a sensitivity of 80%-82%, 80-84%, 80-86%, 80-88%, 80-90%, 80-92%, 80-94%, 80-95%, 80-96%, 80-97%, 80-98%, 80-99%, 80-100%, 82-84%, 82-86%, 82-88%, 82-90%, 82-92%, 82-94%, 82-95%, 82-96%, 82-97%, 82-98%, 82-99%, 82-100%, 84-86%, 84-88%, 84-90%, 84-92%, 84-94%, 84-95%, 84-96%, 84-97%, 84-98%, 84-99%, 84-100%, 86-88%, 86-90%, 86-92%, 86-94%, 86-95%, 86-96%, 86-97%, 86-98%, 86-99%, 86-100%, 88-90%, 88-92%, 88-94%, 88-95%, 88-96%, 88-97%, 88-98%, 88-99%, 88-100%, 90-92%, 90-94%, 90-95%, 90-96%, 90-97%, 90-98%, 90-99%, 90-100%, 92-94%, 92-95%, 92-96%, 92-97%, 92-98%, 92-99%, 92-100%, 94-95%, 94-96%, 94-97%, 94-98%, 94-99%, 94-100%, 95-96%, 95-97%, 95-98%, 95-99%, 95-100%, 96-97%, 96-98%, 96-99%, 96-100%, 97-98%, 97-99%, 97-100%, 98-99%, 98-100%, or 99-100%.

The method provided herein can comprise a specificity that would be amenable for its use a diagnostic to determine whether a subject has been previously exposed to a pathogen as previously described herein. By “specificity,” it is meant that the proportion of negatives that are correctly identified (i.e., subjects who are correctly identified to have not been previously exposed to a pathogen as described herein). In some cases, it may be referred to as a true negative rate. In some embodiments, the method of the present disclosure comprises a specificity of at least 80%, 82%, 84%, 86%, 88%, 90%, 92%, 94%, 95%, 96%, 97%, 98%, 99%, or 100%. In some embodiments, the method of the present disclosure comprises a specificity of 80%-82%, 80-84%, 80-86%, 80-88%, 80-90%, 80-92%, 80-94%, 80-95%, 80-96%, 80-97%, 80-98%, 80-99%, 80-100%, 82-84%, 82-86%, 82-88%, 82-90%, 82-92%, 82-94%, 82-95%, 82-96%, 82-97%, 82-98%, 82-99%, 82-100%, 84-86%, 84-88%, 84-90%, 84-92%, 84-94%, 84-95%, 84-96%, 84-97%, 84-98%, 84-99%, 84-100%, 86-88%, 86-90%, 86-92%, 86-94%, 86-95%, 86-96%, 86-97%, 86-98%, 86-99%, 86-100%, 88-90%, 88-92%, 88-94%, 88-95%, 88-96%, 88-97%, 88-98%, 88-99%, 88-100%, 90-92%, 90-94%, 90-95%, 90-96%, 90-97%, 90-98%, 90-99%, 90-100%, 92-94%, 92-95%, 92-96%, 92-97%, 92-98%, 92-99%, 92-100%, 94-95%, 94-96%, 94-97%, 94-98%, 94-99%, 94-100%, 95-96%, 95-97%, 95-98%, 95-99%, 95-100%, 96-97%, 96-98%, 96-99%, 96-100%, 97-98%, 97-99%, 97-100%, 98-99%, 98-100%, or 99-100%.

Kits provided herein may further comprise controls, for example, positive and negative controls. One or more positive and/or negative control antigens may be supplied in the kit, for example, non-analyte antigens (such antigens from other coronaviruses for specific detection of immune response to SARS-CoV-2). One or more positive and/or negative control antigens may also be supplied in the kit, for example, primers for amplification of non-analyte nucleic acids, such as nucleic acids encoding a house keeping gene product. Exemplary positive controls may include positive samples or standards, e.g. samples obtained from a positive SARS-CoV-2 sample. One of skill in the art can select suitable positive and negative controls for the assays disclosed herein.

In some examples, the kit includes one or more compounds for detecting an amplification product, such as pH-sensitive indicator dye, a DNA intercalator (e.g., propidium iodide, SYBR green or PICOGREEN fluorescent dyes) a chromogenic or colorimetric reagent (such as hydroxynaphthol blue), or a fluorescent indicator (such as calcein).

Isolation or quantification of an analyte may involve lysis of cells in a sample. For example, extracting nucleic acids, e.g. from cells in a sample may involve cell lysis, which may or may not be followed by nucleic acid purification. Cell lysis disrupts the cell and nuclear membranes, releasing the genetic material, e.g. DNA or mRNA. In some embodiments, the kit includes a lysis detergent, such as sodium dodecyl sulfate. The nucleic acids may then be purified with an alcohol precipitation step, usually ice-cold ethanol or isopropanol, or via solid phase purification, e.g., on a silica matrix in a column, resin or on paramagnetic beads in the presence of high concentrations of a chaotropic salt, prior to washing and then elution in a low ionic strength buffer. Other lysis methods include but are not limited to mechanical lysis via ultrasonic vibration and thermal lysis where the sample is heated to 94° C. to disrupt cell membranes.

In additional embodiments, the kits further include primer sets and instructions for amplification and detection of a pathogen. For example, a kit may include primer sets for amplification and detection of a SARS-CoV-2 RNA for indication of SARS-CoV-2 in a subject. In some embodiments, the kit further includes instructions for quantifying analytes as described herein.

In some embodiments, analyzing and quantifying is performed with computing devices. In some embodiments, methods provided herein comprise analyzing and quantifying indicators generated by detection of the sample obtained from a subject and may further comprise providing a result to the subject via the computing devices. In some embodiments, the computing device is an electronic device. In some embodiments, the analyzing and displaying is performed by a single computing device (e.g., smartphone, tablet). In some embodiments, analyzing and displaying is performed at the point of need.

In some embodiments, the methods described herein comprise determining whether a subject has been exposed to a pathogen. In some embodiments, the method comprises determining whether a subject has had an infection by a pathogen (e.g., virus) described herein. In some embodiments, the pathogen is a virus. In some embodiments, the virus is lytic. In some embodiments, the virus is latent. In some embodiments, the pathogen is SARS-CoV-2. In some embodiments, the subject is identified as a carrier of SARS-CoV-2. In some cases, the subject is identified as not being a carrier of SARS-CoV-2.

In some embodiments, the methods described herein comprise determining whether a subject developed an immune response to a pathogen. In some embodiments, the methods described herein comprise determining whether a subject developed immunity to a pathogen. In some embodiments, the method comprises determining a likelihood of severe symptoms of a disease or condition related to a pathogen if exposed to the pathogen (e.g., virus) described herein. In some embodiments, the pathogen is a virus. In some embodiments, the virus is lytic. In some embodiments, the virus is latent. In some embodiments, the pathogen is SARS-CoV-2. In some embodiments, the subject is identified as having a high likelihood of immune to SARS-CoV-2. In some embodiments, the subject is identified as having a high likelihood of developing mild symptoms if exposed to SARS-CoV-2. In some embodiments, the method comprises determining whether a vaccine administered to the subject is effective to immunize the subject against a pathogen, e.g. a virus. In some embodiments, the method comprises determining whether a vaccine administered to the subject is effective to immunize the subject against SARS-CoV-2.

In some embodiments, methods screen for an immune response that is induced by any one of the immunity pathways in the subject. The immune response described herein, in some embodiments, is an innate immune response, an adaptive immune response, passive immune response, or an immune response acquired through immunization. In some embodiment, the method screens for an adaptive immunity.

A subject described herein may be an animal. In some embodiments, the subject is a mammal, such as a human. In some embodiments, the subject is a primate. In some embodiments, the subject is a livestock animal. In some embodiments, the subject is a sheep, cow, pig, horse, donkey, camel, or goat. In some embodiments, the subject is a laboratory test animal. In some embodiments, the subject is a mouse, rat, rabbit, guinea pigs, or hamsters. In some embodiments, the subject is a dog. In some embodiments, the subject is a cat. In some embodiments, the subject is a non-human primate.

In some embodiments, the analysis comprises detecting and measuring signs of the immune response induced by the composition. For example, it can be determined whether a subject has previously been exposed to the tested antigen or an antigen which shows cross-reactivity thereto and thus generated immunological reactivity to the antigen. It can also be determined whether a pathogen or a fragment of a pathogen is currently present in subject. Accordingly, the methods described herein comprises determining or providing a likelihood of whether the subject has an active, a recent, or a latent infection of a pathogen, e.g., a virus. In some embodiments, the methods comprise determining or providing a likelihood of whether a subject is responding to treatment, is going to develop an infection or disease associated with a pathogen, e.g. a virus. In some embodiments, the methods comprise determining or providing a likelihood of whether a subject is immune-suppressed. In some embodiments, the subject is immunocompromised. In some embodiments, the subject has cancer.

In some embodiments, the methods described herein comprise receiving information regarding one or more symptoms from a subject. In some embodiments, the methods described herein comprise receiving information about a diagnosis or prognosis of a disease or condition associated with a pathogen, e.g. a virus infection, from a medical professional. In some instances, the methods include the personal or medical history of the subject as part of the analysis. Personal and medial information of the subject includes age, sex, gender, race or ethnicity, weight, height, body mass index (BMI), heart rate (e.g. ECG and/or peripheral pulse rate), blood pressure, body temperature, respiration rate, past checkups, treatments or therapies, drugs administered, observations, vaccinations, current and/or past symptoms (e.g. fever, vomiting, cough, etc.), known health conditions (e.g. allergies), known diseases or disorders, health history (e.g. past diagnoses), lab test results (e.g. blood test), lab imaging results (e.g. X-rays, MRIs, etc.), genetic information (e.g. known genetic abnormalities associated with disease), family medical history, or any combination thereof.

In some embodiments, the methods comprise analyzing inputs provided by the subject or the healthcare professionals and generating a report comprising suggestions or treatment options based at least partially on the analysis of the inputs. In some instances, the inputs comprise observations made by the healthcare professionals. In some cases, the inputs comprise self-assessment such as pain or discomfort level of the subject. In some cases, the inputs comprise observation and assessment made by both the subject and healthcare professionals. In some embodiments, the inputs comprise communications between the subject and the healthcare professionals or between healthcare professionals. In some cases, the inputs comprise direction, instruction, or warning provided by the healthcare professionals. In some instances, inputs comprise usage and oversight as recorded from the subject using the compositions, methods, and kits described herein.

### Examples

The following examples are included for illustrative purposes only and are not intended to limit the scope of the disclosure.

### Example 1: Collection of Convalescent Blood from Subjects

Blood samples (3 ml) were collected from six asymptomatic unexposed, IgG-negative donors and from six convalescent, IgG-positive donors (an IgG-positive bands on an FDA approved lateral flow assay).

For each donor, whole blood was collected into two sterile tubes containing 45 USP units of lithium heparin. The tubes also contained 4 mg glucose, 12 mg sodium chloride, 4 mg sodium bicarbonate, 3.024 mg disodium phosphate, 800 ug potassium chloride, 200 ug magnesium sulfate, and 200 ug mg calcium nitrate. Amino acids (600 ug glutamine; 400 ug arginine; 100 ug each asparagine, cystine, leucine, and isoleucine; 80 ug lysine hydrochloride; 60 ug serine; 40 ug each aspartic acid, glutamic acid, hydroxyproline, proline, threonine, tyrosine, and valine; 30 ug each histidine, methionine, and phenylalanine; 20 ug glycine; 10 ug tryptophan; and 2 ug reduced glutathione). The following vitamins (70 ug inositol; 6 ug choline chloride; 2 ug each para-aminobenzoic acid, folic acid, nicotinamide, pyridoxine hydrochloride, and thiamine hydrochloride; 0.50 ug calcium pantothenate; 0.4 ug each biotin and riboflavin; and 0.01 ug cyanocobalamin.

One of the blood tubes from each donor also contained COVID-19 mosaic antigen (2 ug per tube). The nucleocapsid protein (N-protein) is a structural protein that binds to the coronavirus RNA genome, thus creating a shell (or capsid) around the enclosed nucleic acid. The E. coli derived recombinant protein used in the test contains the Coronavirus 2019 full length nucleocapsid Mosaic immunodominant regions.

The tubes were incubated at 37° C. on a rotary mixer for 24 hours. At the end of this period, the tubes were collected and spun at 3200 rpm (1000 g) for 15 minutes.

The plasma was collected for metabolomics analysis and 0.5 ml of buffy coat was collected from the top of the packed cell layer.

The plasma and buffy coats were frozen for subsequent analysis.

### Example 2: Measurement of Interferon-Gamma Response

The interferon-gamma response of the buffy coat was measured in each tube by extracting RNA from the samples. The extraction was performed with the use of a guanidinium based lysis solution, a phenol/chloroform extraction, and RNA purification on a glass fibre filter. The phenol/chloroform extraction step removes proteins from the sample to prevent clogging of the glass fibre filters and eliminate heme contamination. A post-elution DNase treatment was performed (RiboPure™ RNA Purification Kit, blood, ThermoFischer).

Loop-mediated isothermal proliferation (LAMP) technique was used to amplify in conjunction with nanopore technology. The technique that replicates the DNA with high specificity, efficiency, and speed. IFNg-specific LAMP was carried out in a 25-μl reaction mixture containing 50 pmol each of the FIP and BIP primers, 5 pmol each of the outer F3 and B3 primers, 25 pmol each of the loop primers F and B, and 8 U of the large Bst DNA polymerase fragment (New England BioLabs, Beverly, MA) in 20 mM Tris-HCl (pH 8.8), 10 mM KCl, 8 mM MgSO4 10 mM (NH4)2SO4, 0.1% Tween 20, 0.8 M betaine (Sigma, St. Louis, MO), 1.4 mM each deoxynucleoside triphosphate (dNTP), and the template DNA. The results were visually confirmed through fluorescence (see image) after 30 minutes at 65° C. followed by 80° C. for 5 minutes.

The outcome of the LAMP assay was quantified using nanopore technology. Sequencing libraries were generated. 2 μl of sequencing Hairpin HP Adapter (HPA) were ligated using 50 μl Blunt/TA Ligase Master Mix (New England Biolabs, Ipswich, USA) in the presence of 10 μl Adapter Mix and incubated for 10 minutes at room temperature. The adapter mix HPA consists of a linear double strand sequence and a hairpin sequence that links the positive and negative strand of each fragment to allow the sequencing of both strands (2D reads). After adapter ligation, the library was conjugated with 1 μl of Hairpin Tether (HPT) motor protein to allow the passage of the fragment through the nanopore on the flowcell.

Previous exposure to the virus was determined by assessing the difference in the background IFN-gamma response from blood cultured with and without the COVID-19 mosaic antigen.

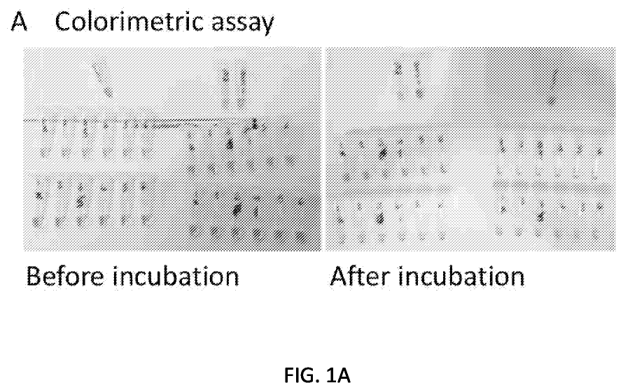

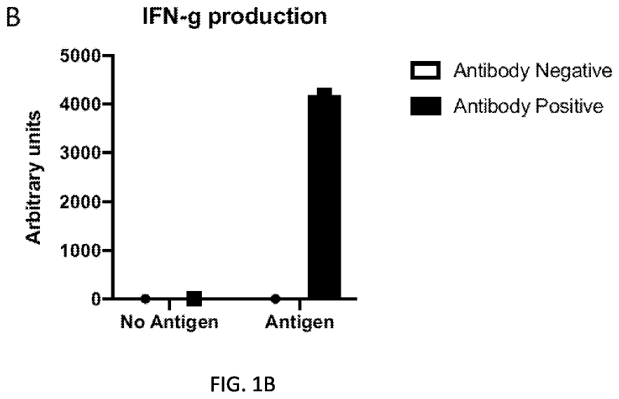

The blood samples of those collected from asymptomatic unexposed, IgG-negative donors showed little or no response to the antigen, but convalescent samples from IgG-positive donors (an IgG-positive bands on an FDA approved lateral flow assay) showed elevated production of IFN-g (FIGS. 1A-1B).

### Example 3: Detection of Interferon Gamma Response

### RT PCR Protocol

Buffy Coat samples were extracted using the ZymoResearch Quick-RNA Whole Blood Kit (R1201, zymoresearch). Samples were extracted at varying times after sample receipt. Received samples were stored at −80 until extraction. After extraction samples proceeded to downstream analysis as soon as possible. Extracted RNA was stored at −80 until use if necessary.

cDNA was prepared using the RNA to cDNA EcoDry Premix (639549, Takara Bio). TaqMan assays were established for interferon-gamma (IFNG—test target), Actin (ACTB—control gene) and GAPDH (a second control gene for validation). TaqMan assays for IFNG, ACTB and GAPDH were selected as follows: TaqMan™ Gene Expression Assay (FAM) Catalog Number: 4331182 Unit Size: S (250 reactions/250 μL), inventoried Assay ID: Hs00989291_m1 Gene Symbol: IFNG TaqMan™ Gene Expression Assay, VIC primerlimited Catalog Number: 4448484 Unit Size: S (360 reactions), made to order Assay ID: Hs02758991_g1 Gene Symbol: GAPDH; TaqMan™ Gene Expression Assay, VIC primerlimited Catalog Number: 4448484 Unit Size: S (360 reactions), made to order Assay ID: Hs01060665_g1 Gene Symbol: ACTB Samples were analysed using an ABI7500-fast as per manufacturer's instructions.

Using the previously described protocol of Example 1 to obtain whole blood and measure interferon gamma response, seven whole blood samples from COVID positive patients and seven whole blood samples from COVID negative patients were incubated with the CoV2 mosaic antigen for 24 hours, and the samples quantified for expression of IFN-gamma using RT-PCR.

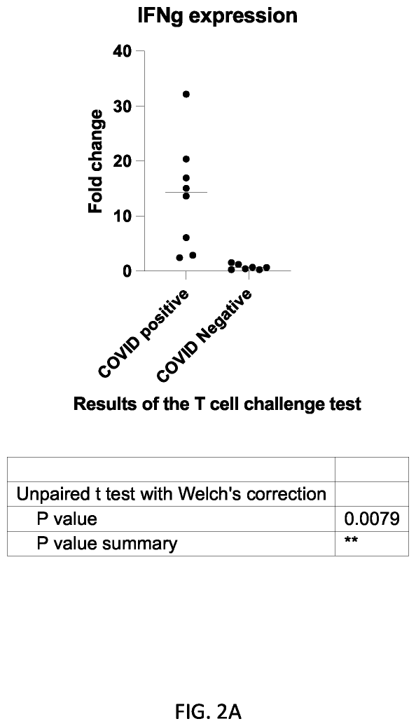

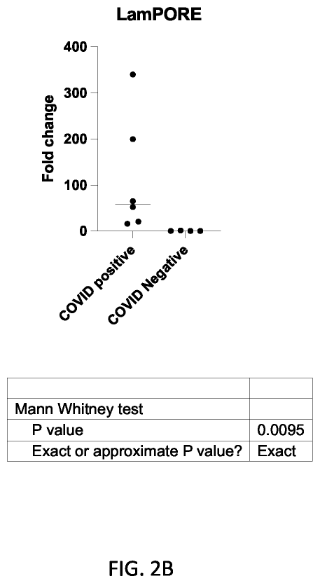

The blood samples from COVID negative patients showed little or no response to antigen, whereas samples from COVID positive patients showed robust expression of IFN-gamma (FIG. 2A). It was found that using an unpaired T-test with Welch's correction, a p-value of 0.0079 (**, P<0.01) was attained between the two sample groups (FIG. 2A), demonstrating that differences in IFN-gamma expression can be used to determine prior exposure to COVID. In addition, the lowest fold change detected in a sample for positive samples was 2.43 change, whereas the highest fold change detected in the negative samples was 1.5 fold change, thereby providing a sufficient difference in changes in expression in positive and negative samples.

Using the previously described protocol of Example 1 to obtain whole blood and measure interferon gamma response, eight whole blood samples from COVID positive patients and seven whole blood samples from COVID negative patients were incubated with the CoV-2 mosaic antigen for 24 hours, and the samples quantified for expression of IFN-gamma using LAMP combined with Nanopore technology (LamPORE) as described above. The blood samples from COVID negative patients showed little or no response to antigen, whereas samples from COVID positive patients showed robust expression of IFN-gamma (FIG. 2B). It was found that using a Mann-Whitney test, a p-value of 0.0095 was attained between the two sample groups (FIG. 2B), demonstrating that differences in IFN-gamma expression can be used to determine prior exposure to COVID.

### Other CoV-2 Antigens

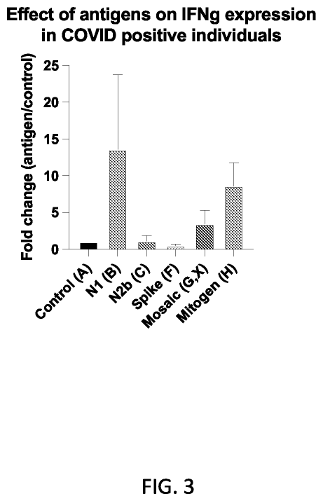

To determine whether exposure to COVID can be determine using the above method with antigens other than the mosaic CoV-2 antigen, whole blood samples from eight COVID positive individuals were assessed. In FIG. 3, COVID proteins N1, N2b, full length Spike and a COVID mosaic antigen were incubated with whole blood samples for 24 hours. Adjuvant alone (control) was used as a negative control. Mitogen is pokeweed antigen, which is used as a positive control as it activates the T-cell response. In each antigen or control sample, IFN-gamma expression was quantified using RT-PCR. The N1 antigen and COVID mosaic antigen elicited the greatest fold change in IFN-gamma expression (FIG. 3). The N1 peptide is a polypeptide including residues 1-183 AA from SEQ ID NO 1. N2b is a polypeptide including residues 166-422 of SEQ ID NO: 1.

Preliminary analysis of Lactate levels corroborated the findings. Lactate is released by rapidly proliferating T cells.

### Example 4: Time Course of T Cell Challenge Test

To determine if a shorter incubation time with the antigen can be used, a control antigen or a COVID mosaic antigen was incubated with whole blood for different amounts of time (2, 4 or 6 hours) and the analyte was measured by RT-PCR.

The COVID mosaic antigen was incubated in whole blood from eight COVID positive donors and IL-2 or IFN-gamma were measured by RT-PCR. The control contains only adjuvant.

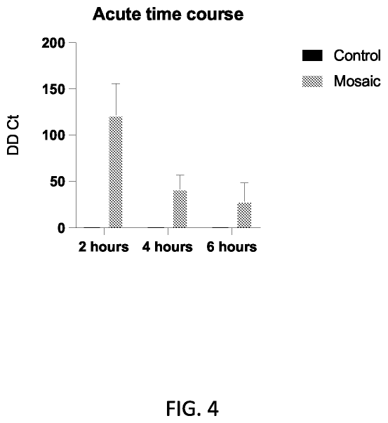

In COVID positive convalescent donors, IFN-gamma response is detectable in samples that were incubated with the mosaic antigen for at 2 hours, and signal is still detectable at 4 and 6 hours of incubation with the antigen. (FIG. 4). After 2 hours of antigen incubation, the sample incubated with the mosaic antigen exhibited a DD Ct value of greater than 100. The control samples were tested, but the values could not be resolved.

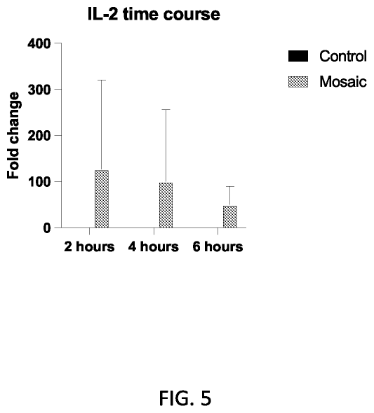

In COVID positive convalescent donors, IL-2 expression is detectable in samples that were incubated with the mosaic antigen for at 2 hours, and signal is still detectable at 4 and 6 hours of incubation with the antigen. (FIG. 5). The control samples were tested, but the values could not be resolved. The IL-2 primers utilized were forward primer: 5′-CCTGAGCAGGGAGAATTACA-3′ (SEQ ID NO: 60), reverse primer: 5′-TCCAGAACATGCCGCAGA-3′ (SEQ ID NO: 61).

Thus, showing that incubation with the COVID antigens can be shortened and still yield a detectable signal to determine prior exposure in a patient sample.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A container for detecting an immune response to a pathogen by a subject, wherein said container comprises an antigen of said pathogen and an anticoagulation agent, and wherein said antigen comprises a specific T cell recognized epitope of said pathogen.

2. The container of claim 1, wherein said antigen is lyophilized.

3. The container of claim 1, wherein said antigen is in a solution.

4. The container of any one of the preceding claims, further comprising a sample obtained from said subject.

5. The container of claim 4, wherein said sample comprises PBMCs, CD8 T cells, NK cells, CD4 T cells, B cells, or any combination thereof.

6. The container of claim 4 wherein said sample comprises a body fluid.

7. The container of claim 6, wherein said sample comprises saliva.

8. The container of claim 6, wherein said sample comprises whole blood.

9. The container of any one of the preceding claims, wherein said subject is a human.

10. The container of claim 9 for use in detecting an immune response to said pathogen for a subject that exhibits symptoms of a condition associated with exposure to said pathogen.

11. The container of claim 9 for use in detecting an immune response to said pathogen for a subject that exhibits symptoms of a condition associated with exposure to said pathogen.

12. The container of claim 9 for use in detecting an immune response to said pathogen for a subject suspected to have been exposed to said pathogen.

13. The container of any one of the preceding claims for use in incubating whole blood for at least 30 minutes.

14. The container of any one of the preceding claims for use in incubating whole blood for at least 6 hours.

15. The container of any one of the preceding claims for use in incubating whole blood for at least 24 hours.

16. The container of any one of the preceding claims, wherein said antigen is stable for at least 30 days.

17. The container of claim 16, wherein said antigen is stable for at least 6 months.

18. The container of claim 17, wherein said antigen is stable for at least a year.

19. The container of any one of the preceding claims, wherein said anticoagulation agent is heparin, citrate, unfractionated heparin, bivalirudin, or hirudin.

20. The container of claim 19, wherein said anticoagulation agent is lithium heparin.

21. The container of any one of the preceding claims, wherein said pathogen is a mammalian pathogen.

22. The container of claim 21 wherein said pathogen is a human pathogen.

23. The container of claim 21 wherein said pathogen is an animal pathogen.

24. The container of any one of the preceding claims, wherein said pathogen is a viral pathogen and said antigen is from a disease causing virus.

25. The container of claim 24, wherein said pathogen is a mammalian viral pathogen.

26. The container of claim 24, wherein said pathogen is a human or animal viral pathogen.

27. The container of any one of claims 24-26, wherein said antigen is from a DNA virus.

28. The container of any one of claims 24-26, wherein said antigen is from a RNA virus.

29. The container of claim 28, wherein said antigen is from a retrovirus.

30. The container of claim 28, wherein said antigen is from a Coronavirus, a Rhabdovirus, a paramyxovirus, an influenza virus, a respiratory syncytial virus, a cytomegalovirus, a Hepatitis virus or a orthomyxovirus.

31. The container of claim 30, wherein said antigen is from a SARS-CoV2 virus.

32. The container of claim 31, wherein said antigen comprises a polypeptide comprising a sequence with at least 80% identity to SEQ ID NO: 1.

33. The container of claim 31, wherein said antigen comprises a polypeptide comprising a sequence with at least 85% identity to SEQ ID NO: 1.

34. The container of claim 31, wherein said antigen comprises a polypeptide comprising a sequence with at least 90% identity to SEQ ID NO: 1.

35. The container of any one of claims 31-34, wherein said antigen comprises a polypeptide comprising a sequence with at least 80% identity to SEQ ID NO: 2.

36. The container of claim 35, wherein said antigen further comprises a polypeptide comprising a sequence with at least 85% identity to SEQ ID NO: 2.

37. The container of claim 36, wherein said antigen further comprises a polypeptide comprising a sequence with at least 90% identity to SEQ ID NO: 2.

38. The container of any one of claims 31-37, wherein said antigen comprises a peptide comprising 5-14 consecutive amino acids of SEQ ID NO: 1.

39. The container of any one of claims 31-38, wherein said antigen comprises a peptide comprising 5-14 consecutive amino acids of SEQ ID NO: 2.

40. The container of any one of claims 31-37, wherein said antigen comprises a peptide comprising at least 15 consecutive amino acids of SEQ ID NO: 1.

41. The container of any one of claims 31-38, wherein said antigen comprises a peptide comprising at least 15 consecutive amino acids of SEQ ID NO: 2.

42. The container of any one of the preceding claims, wherein said antigen comprises at least two specific T cell recognized epitopes of said virus.

43. The container of any one of the preceding claims, wherein said T cell recognition is by a CD4+ T cell.

44. The container of any one of claims 1-42, wherein said T cell recognition is by a CD8+ T cell.

45. The container of any one of the preceding claims, wherein said container further comprises a second antigen comprising a second specific T cell recognized epitope of said pathogen.

46. The container of any one of claims 1-45, wherein said container further comprises a second antigen of a second pathogen, and wherein said second antigen comprises a specific T cell recognized epitope of said second pathogen.

47. The container of claim 46, wherein said second pathogen is a human pathogen.

48. The container of claim 47, wherein said pathogen is a viral pathogen and second said antigen is from a disease causing virus.

49. The container of claim 48, wherein said second pathogen is a mammalian viral pathogen.

50. The container of claim 49, wherein said second pathogen is a human or animal viral pathogen.

51. The container of any one of the preceding claims, wherein said container further comprises one or more of a sugar, an amino acid, and a vitamin.

52. The container of claim 51 wherein said sugar is a glucose.

53. The container of any one of the preceding claims further comprising an aluminum adjuvant.

54. The container of claim 53, wherein said aluminum adjuvant and said antigen are at a ratio of 1:1 to 3:1 by weight, wherein weight of said aluminum adjuvant is as measured by weight of aluminum ion.

55. A container for detecting an immune response to a SARS-Cov2 virus in a subject, wherein said container comprises:
(i) lithium heparin,
(ii) glucose,
(iii) an aluminum adjuvant,
and
(iv) a SARS-Cov2 antigen that comprises the sequence of SEQ ID NO: 2,
wherein said aluminum adjuvant and said SARS-Cov2 antigen are at a ratio of 1:1 to 3:1 by weight,
wherein weight of said aluminum adjuvant is as measured by weight of aluminum ion.

56. A kit for detecting an immune response to a virus in a subject comprising the container of any one of the preceding claims and a second container comprising an anticoagulation agent and not said antigen.

57. The kit of claim 56, further comprising a detection agent for detecting an immune cell response to said antigen, wherein said detection agent quantifies an indicator of said immune cell response.

58. The kit of claim 57, wherein said indicator comprises an amount of an immune-response analyte.

59. The kit of claim 58, wherein said indicator comprises a ratio of two or more immune-response analytes.

60. The kit of claim 58 or 59, wherein said immune-response analyte or said two or more immune-response analytes are immunoproteins.

61. The kit of claim 60 wherein said immune-response analyte or said two or more immune-response analytes are nucleic acids encoding said immunoproteins.

62. The kit of claim 57, wherein said indicator is an immune cell proliferation indicator.

63. The kit of claim 62, wherein said immune-response analyte is an IFN-gamma protein or a nucleic acid encoding an IFN-gamma protein.

64. The kit of claim 63, further comprising one or more primers that hybridize with said nucleic acid encoding said IFN-gamma protein.

65. The kit of any one of claims 56-64 further comprising a lysis agent.

66. The kit of any one of claims 56-65 further comprising instructions for quantifying said immune-response analytes.

67. The kit of any one of claims 56-66 further comprising a set of primers that hybridize with a nucleic acid of said pathogen.

68. The kit of claim 67, wherein said set of primers hybridize with a nucleic acid encoding a SARS-CoV-2 protein.

69. A kit for detecting an immune response to a SARS-Cov2 virus by a subject, wherein said kit comprises:
a) a first container comprising (i) lithium heparin (ii) glucose, (iii) an aluminum adjuvant, and (iv) a SARS-Cov2 antigen that comprises the peptide sequence of SEQ ID NO: 2,
b) a second container comprising lithium heparin, glucose, and not said SARS-Cov2 antigen, wherein said first and second containers are sterile glass or polycarbonate containers, and
c) a set of primers that comprise a sequence selected from Table 2.
wherein said aluminum adjuvant and said SARS-Cov2 antigen are at a ratio of 1:1 to 3:1 by weight,
wherein weight of said aluminum adjuvant is as measured by weight of aluminum ion.

70. A method for detecting an immune response to a pathogen by a subject, comprising:
i) incubating a blood sample from said subject in a container comprising an antigen and an anticoagulation agent, wherein said antigen comprises a specific T cell recognized epitope of said pathogen, and
ii) incubating a second blood sample from said subject in a second container comprising said anticoagulation agent in (i) and not said antigen,
iii) quantifying an amount of an immune-response analyte in said container and said second container,
iv) comparing said amount of said immune-response analyte in said container and said second container to determine whether the subject has an immune response to said pathogen.

71. The method of claim 70 wherein said quantifying in iii) comprises using mass spectrometry to determine said amount of said immune-response analyte.

72. The method of claim 70 or 71 wherein said immune-response analyte is a protein.

73. The method of claim 72, further comprising isolating said immune response analyte.

74. The method of claim 73, further comprising isolating a nucleic acid encoding said immune response analyte.

75. The method of claim 71, further comprising isolating total RNA from said container.

76. The method of any one of claims 72-75, wherein said quantifying in (iii) comprises Western blot, mass spectrometry assay, UV spectrometry assay, aptamer based assay, or colorimetric assay.

77. The method of claim 74, wherein said quantifying in (iii) comprises amplifying said nucleic acid encoding said immune-response marker.

78. The method of claim 77, wherein said quantifying in (iii) comprises determining the amount of said nucleic acid encoding said immune-response marker with loop-mediated isothermal proliferation (LAMP) assay, RNA seq, or quantitative RT-PCR.

79. The method of claim 78, wherein said quantifying in (ii) further comprises analysis using a nanopore technology.

80. The method of any one of claims 70-79, wherein said container further comprises a second antigen comprising a specific T cell recognized epitope of a second pathogen, wherein said second container does not comprise said second antigen, and wherein said comparing in (iv) comprises comparing said amount of said immune-response analyte in said container and said second container to determine whether the subject has an immune response to said second pathogen.

81. The method of any one of claims 70-80, further comprising v) quantifying an amount of a second immune-response analyte in said container and said second container, and vi) comparing said amount of said second immune-response analyte in said container and said second container to determine whether said subject has an immune response to said pathogen.

82. The method of claim 81, wherein said comparing in vi) comprises comparing a ratio of said immune-response analyte to said second immune-response analyte in said container and said second container to determine whether said subject has an immune response to said pathogen.

83. The method of any one of claims 70-82, further comprising vii) detecting said pathogen or said second pathogen in said sample, wherein said detecting comprises amplifying a nucleic acid of said pathogen or said second pathogen.

84. The method of claim 83, wherein said amplifying comprises LAMP assay, RNA-Seq, or RT-PCR.

85. The method of claim 84, wherein said amplifying is with a set of primers, wherein said set of primers hybridize with said nucleic acid.

86. The method of claim 85, wherein said set of primers hybridize with a nucleic acid encoding a SARS-CoV-2 protein.

87. A method for detecting an immune response to a SARS-Cov2 virus by a subject, comprising:
i) incubating a whole blood sample from said subject in a container comprising lithium heparin, glucose, an aluminum adjuvant, and a specific SARS-Cov2 antigen for at least 24 hours,
ii) incubating a second whole blood sample from said subject in a second container comprising lithium heparin, glucose, an aluminum adjuvant, and not a SARS-Cov2 antigen, and
iii) quantifying an amount of an IFN-gamma mRNA in said container and said second container to determine whether said subject has an immune response to said SARS-Cov2 virus,
wherein said aluminum adjuvant and said SARS-Cov2 antigen are at a ratio of 1:1 to 3:1 by weight,
wherein weight of said aluminum adjuvant is as measured by weight of aluminum ion,
wherein said SARS-Cov2 antigen comprises the peptide sequence of SEQ ID NO: 2,
and wherein said quantifying in (iii) comprises amplifying said IFN-gamma mRNA with loop-mediated isothermal proliferation (LAMP) assay using primers selected from Table 2.


SEQUENCE LISTING (61 sequences)

SEQ ID NO: 1 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 359 aa, Artificial Sequence
  source: /note=“Description of Artificial Sequence Synthetic polypeptide”
ADPEFRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPHHHHHH

SEQ ID NO: 3 - protein, 180 aa, Severe acute respiratory syndrome coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 4 - protein, 638 aa, Severe acute respiratory syndrome coronavirus 2
AYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGG

SEQ ID NO: 5 - protein, 1945 aa, Severe acute respiratory syndrome coronavirus 2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 6 - protein, 500 aa, Severe acute respiratory syndrome coronavirus 2
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 7 - protein, 306 aa, Severe acute respiratory syndrome coronavirus 2 [sequence omitted]

SEQ ID NO: 8 - protein, 290 aa, Severe acute respiratory syndrome coronavirus 2
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 9 - protein, 83 aa, Severe acute respiratory syndrome coronavirus 2
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 10 - protein, 198 aa, Severe acute respiratory syndrome coronavirus 2
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 11 - protein, 113 aa, Severe acute respiratory syndrome coronavirus 2
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 12 - protein, 139 aa, Severe acute respiratory syndrome coronavirus 2
AGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQ

SEQ ID NO: 13 - protein, 43 aa, Severe acute respiratory syndrome coronavirus 2
MIELSLIDFYLCFLAFLLFLVLIMLIIFWFSLELQDHNETCHA

SEQ ID NO: 14 - protein, 4405 aa, Severe acute respiratory syndrome coronavirus 2 [sequence omitted]

SEQ ID NO: 15 - protein, 180 aa, Severe acute respiratory syndrome coronavirus 2; identical to SEQ ID NO: 3

SEQ ID NO: 16 - protein, 638 aa, Severe acute respiratory syndrome coronavirus 2; identical to SEQ ID NO: 4

SEQ ID NO: 17 - protein, 1945 aa, Severe acute respiratory syndrome coronavirus 2; identical to SEQ ID NO: 5

SEQ ID NO: 18 - protein, 500 aa, Severe acute respiratory syndrome coronavirus 2; identical to SEQ ID NO: 6

SEQ ID NO: 19 - protein, 306 aa, Severe acute respiratory syndrome coronavirus 2 [sequence omitted]

SEQ ID NO: 20 - protein, 290 aa, Severe acute respiratory syndrome coronavirus 2; identical to SEQ ID NO: 8

SEQ ID NO: 21 - protein, 83 aa, Severe acute respiratory syndrome coronavirus 2; identical to SEQ ID NO: 9

SEQ ID NO: 22 - protein, 198 aa, Severe acute respiratory syndrome coronavirus 2; identical to SEQ ID NO: 10

SEQ ID NO: 23 - protein, 113 aa, Severe acute respiratory syndrome coronavirus 2; identical to SEQ ID NO: 11

SEQ ID NO: 24 - protein, 139 aa, Severe acute respiratory syndrome coronavirus 2; identical to SEQ ID NO: 12

SEQ ID NO: 25 - protein, 932 aa, Severe acute respiratory syndrome coronavirus 2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 26 - protein, 601 aa, Severe acute respiratory syndrome coronavirus 2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 27 - protein, 527 aa, Severe acute respiratory syndrome coronavirus 2
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 28 - protein, 346 aa, Severe acute respiratory syndrome coronavirus 2
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 29 - protein, 298 aa, Severe acute respiratory syndrome coronavirus 2
SSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 30 - protein, 13 aa, Severe acute respiratory syndrome coronavirus 2
SADAQSFLNGFAV

SEQ ID NO: 31 - protein, 38 aa, Severe acute respiratory syndrome coronavirus 2
MGYINVFAFPFTIYSLLLCRMNSRNYIAQVDVVNFNLT

SEQ ID NO: 32 - protein, 7096 aa, Severe acute respiratory syndrome coronavirus 2 [sequence omitted]

SEQ ID NO: 33 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 34 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 35 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 36 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 37 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 38 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 39 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 40 - DNA, 25 bp, Artificial Sequence
  source: /note=“Description of Artificial Sequence Synthetic primer”
tcaatatagaaccgaaaagtcgaga

SEQ ID NO: 41 - DNA, 24 bp, Artificial Sequence
  source: /note=“Description of Artificial Sequence Synthetic primer”
caacttctttggcttaattctctc

SEQ ID NO: 42 - DNA, 22 bp, Artificial Sequence
  source: /note=“Description of Artificial Sequence Synthetic primer”
gcttgatacaagaactactgat

SEQ ID NO: 43 - DNA, 20 bp, Artificial Sequence
  source: /note=“Description of Artificial Sequence Synthetic primer”
ctgttactgccaggacccat

SEQ ID NO: 44 - DNA, 19 bp, Artificial Sequence
  source: /note=“Description of Artificial Sequence Synthetic primer”
tccagtaagtctacatcgc

SEQ ID NO: 45 - DNA, 21 bp, Artificial Sequence
  source: /note=“Description of Artificial Sequence Synthetic primer”
ttaccttgagaaaagaatccg

SEQ ID NO: 46 - DNA, 24 bp, Artificial Sequence
  source: /note=“Description of Artificial Sequence Synthetic primer”
gaactactgatttcaacttctttg

SEQ ID NO: 47 - DNA, 23 bp, Artificial Sequence
  source: /note=“Description of Artificial Sequence Synthetic primer”
gcattatttttctgtcactctcc

SEQ ID NO: 48 - DNA, 44 bp, Artificial Sequence
  source: /note=“Description of Artificial Sequence Synthetic primer”
gaacccaaaacgatgcagagccttaattctctcggaaacgatga

SEQ ID NO: 49 - DNA, 46 bp, Artificial Sequence
  source: /note=“Description of Artificial Sequence Synthetic primer”
ctgttactgccaggacccatatgtctttccaattcttcaaaatgcc

SEQ ID NO: 50 - DNA, 24 bp, Artificial Sequence
  source: /note=“Description of Artificial Sequence Synthetic primer”
atatagaaccgaaaagtcgagacg

SEQ ID NO: 51 - DNA, 21 bp, Artificial Sequence
  source: /note=“Description of Artificial Sequence Synthetic primer”
gcaggtcattcagatgtagcg

SEQ ID NO: 52 - DNA, 19 bp, Artificial Sequence
  source: /note=“Description of Artificial Sequence Synthetic primer”
ggatgcaactcctgtcttg

SEQ ID NO: 53 - DNA, 18 bp, Artificial Sequence
  source: /note=“Description of Artificial Sequence Synthetic primer”
gttctgtggccttcttgg

SEQ ID NO: 54 - DNA, 44 bp, Artificial Sequence
  source: /note=“Description of Artificial Sequence Synthetic primer”
ccagttgtagctgtgttttctttgtgcactaagtcttgcacttg

SEQ ID NO: 55 - DNA, 42 bp, Artificial Sequence
  source: /note=“Description of Artificial Sequence Synthetic primer”
agcatttactgctggatttacagatgttgtgagcatcctggt

SEQ ID NO: 56 - DNA, 20 bp, Artificial Sequence
  source: /note=“Description of Artificial Sequence Synthetic primer”
tgcactaagtcttgcacttg

SEQ ID NO: 57 - DNA, 24 bp, Artificial Sequence
  source: /note=“Description of Artificial Sequence Synthetic primer”
ccagttgtagctgtgttttctttg

SEQ ID NO: 58 - DNA, 18 bp, Artificial Sequence
  source: /note=“Description of Artificial Sequence Synthetic primer”
tgttgtgagcatcctggt

SEQ ID NO: 59 - DNA, 24 bp, Artificial Sequence
  source: /note=“Description of Artificial Sequence Synthetic primer”
agcatttactgctggatttacaga

SEQ ID NO: 60 - DNA, 20 bp, Artificial Sequence
  source: /note=“Description of Artificial Sequence Synthetic primer”
cctgagcagggagaattaca

SEQ ID NO: 61 - DNA, 18 bp, Artificial Sequence
  source: /note=“Description of Artificial Sequence Synthetic primer”
tccagaacatgccgcaga